(19) United States
(12) Patent Application Publication
(10) Pub. No.: US 2025/0064978 A1
(43) Pub. Date: Feb. 27, 2025

(54) GENE THERAPY METHODS FOR TREATMENT OF DIABETES

(71) Applicants: Jaguar Gene Therapy, LLC, Lake Forest, IL (US); The Administrators of the Tulane Educational Fund, New Orleans, LA (US)

(72) Inventors: Michael Hughes, London (GB); Sandeep Dhall, Columbia, MD (US); Hongju Wu, New Orleans, LA (US); Vivian A. Fonseca, New Orleans, LA (US)

(21) Appl. No.: 18/715,015

(22) PCT Filed: Nov. 30, 2022
(86) PCT No.: PCT/US2022/080686

§ 371 Date: May 30, 2024

Related U.S. Application Data
(60) Provisional application No. 63/284,392, filed on Nov. 30, 2021.

(51) Int. Cl. A61K 48/00; A61K 38/17; A61P 3/10; C12N 15/86

(52) U.S. Cl. CPC A61K 48/0058; A61K 38/1709; A61K 48/0075; A61P 3/10; C12N 15/86; C12N 2750/14143; C12N 2750/14151; C12N 2830/50

(57) ABSTRACT

Provided are recombinant adeno-associated virus (rAAV) vectors comprising a transgene to express Pax4; virions comprising said vectors (rAAV virions); methods of their production; methods of their use, including methods for treating diabetes, increasing insulin production and transdifferentiating α-cells to β-cells; pharmaceutical compositions and kits including same.

### SEQUENCE LISTING

The contents of the electronic sequence listing submitted herewith as file 38061_0004P1.xml; Size: 64,269 bytes; and Date of Creation: Nov. 30, 2022, is herein incorporated by reference in its entirety.

### FIELD OF THE INVENTION

Provided are viral constructs, particles and compositions for use in the treatment of diabetes mellitus that have a transgene encoding PAX4 for expression in pancreatic cells.

### BACKGROUND

According to the 2020 National Diabetes Statistics Report (the “Report”) published by the Centers for Disease Control and Prevention (“CDC”), the crude estimate of the number of people in the United States with diabetes mellitus (hereafter, “diabetes”) was 34.2 million people of all ages, about 10.5% of the population. Of these, the CDC estimated that 26.9 million people had been diagnosed with diabetes, leaving an estimated 7.3 million people with diabetes but not yet diagnosed with the condition.

The islets of Langerhans (sometimes referred to as “pancreatic islets”), located in the pancreas, contain α-cells, which produce glucagon, and β-cells, which produce insulin, a hormone which assists cells in absorbing glucose from the bloodstream. In type 1 diabetes mellitus (sometimes referred to as “T1DM”), an individual's immune system destroys the individual's β-cells, requiring that individual to take insulin daily for the remainder of their life. Type 2 diabetes (sometimes referred to as “T2DM”) results from an individual's body becoming resistant to the effects of insulin, and the body's inability to produce enough insulin to overcome that resistance. Either condition can result in too much glucose in the blood, with damage over time to multiple organs.

Currently, insulin therapy is the main treatment for T1D. Despite tremendous progresses, insulin therapy has not achieved glycemic control as smoothly and precisely as functional β cells do in response to variations in glucose levels. As a result, insulin therapy does not prevent eventual development of diabetes-related complications that compromise the quality and length of the patients' life.

The Report states that, in the United States in 2017, diabetes was a leading cause of major cardiovascular disease and limb amputations, as well as being the leading cause of end-stage renal disease, the leading cause of new cases of blindness in adults aged 18 to 64 years of age, and the seventh leading cause of death in the country. Therefore, an ultimate treatment for T1D requires β cell therapy—that is, the ability to replace the lost β-cells, not just the hormone insulin.

In the early stages of T1DM, there is decline in beta cells, however the alpha cells are normal, leading to an imbalance between alpha cell and beta cell function, with considerable glucagon excess contributing to several clinical manifestations, such as ketosis. While this imbalance increases over time, alpha cells remain in considerable excess in relation to the declining function of beta cells. In type 2 diabetes, there is also a relative dysfunction of alpha cells, with excess secretion of glucagon in the face of insulin deficiency, though not as severe as in type 1. The defect in alpha cell function that occurs in type 2 diabetes reflects impaired glucose sensing, that is partly corrected by GLP 1 (Dunning B; Diabetologia 2005, 48(9):1700-13); but the disease continues to progress despite exogenous GLP-1, as fundamental abnormalities within the islet remain.

The embryonic development of pancreatic β cells is controlled by sequential expression of a series of transcription factors. One of the transcription factors, Pax4, has been shown to play essential roles in β cell differentiation as well as β cell expansion and survival. Lorenzo et al. (2017), Brun et al., Lorenzo et al. (2015), Sosa-Pineda et al. Ectopic Pax4 expression in glucagon-producing α cells induces their conversion to insulin-producing β cells. Collomat et al. Adenovirus (Ad5)-mediated Pax4 gene delivery directly into the pancreas of T iD mouse models showed some beneficial effects but lacked efficiency. Zhang et al. (2016). Ad5-mediated Pax4 gene delivery into primary islet cells not only promotes β cell survival, but also induces α-to-β cell transdifferentiation in primary islet cells and showed activity when transplanted into mice. Parajuli et al. These data suggest that Pax4 is a candidate target for T1D therapy that aims to preserve and regenerate β cells.

Recent studies have shown therapeutic benefits of inducing α-to-β cell transdifferentiation. Furuyama et al., Xiao et al. Specifically, gene delivery of Pdxl and MafA into pancreas has been shown to normalize blood glucose in both STZ-induced T iD or NOD mice; further examination confirmed α-to-β cell conversion in the treated mice. Xiao et al.

Therapies that would promote β-cell formation for treatment of diabetes would be desirable to thereby reduce symptoms or damage due to such deficiency. The compositions and methods disclosed herein fill these and other needs.

### SUMMARY OF INVENTION

Provided are viral constructs, viral particles and compositions for use in the treatment of diabetes mellitus and preventing or slowing the progression from pre-diabetes to diabetes that have a transgene encoding PAX4 for expression in pancreatic cells. The vector constructs are used to generate recombinant AAV virions or particles which are administered for rAAV gene therapy in a subject suffering from diabetes or at high risk to develop diabetes to deliver to cells, including pancreatic cells, of the subject a nucleic acid encoding Pax4. In some embodiments, expression of Pax4 in islet cells, including α-cells, causes transdifferentiation of the α-cells to β-cells which produce insulin, thereby treating and ameliorating the diabetes and/or reducing the risk of progression from pre-diabetes to diabetes in the subject. In some embodiments, the expression of Pax4 results in maintenance or protection of the β-cells in the subject thereby treating and/or ameliorating and/or slowing the progression of diabetes in the subject.

Provided are AAV vectors comprising an expression cassette which comprises a nucleotide sequence encoding Pax4, including human Pax4, operably linked to one or more regulatory elements that promote expression of the Pax4 coding sequence and a polyadenylation (poly(A)) tail signal; the promoter regulatory element comprising a constitutive promoter, including a cytomegalovirus (CMV) early enhancer/chicken β-actin/rabbit β-globin splice acceptor (CAG) promoter, or a pancreas cell, or alpha cell or beta cell, specific promoter such as a human or rodent insulin promoter or a human or rodent glucagon promoter; and the poly(A) tail signal. In some embodiments, the promoter is a CAG promoter (for example, having a nucleotide sequence of SEQ ID NO: 4). The polyA signal sequence may be a SV40 polyadenylation signal sequence, for example, with a nucleotide sequence of SEQ ID NO: 7. In some embodiments, the promoter is a mouse glucagon promoter (MGP; for example, having a nucleotide sequence of SEQ ID NO: 5) or a rat insulin promoter (RIP; for example, having a nucleotide sequence of SEQ ID NO: 6). The expression cassette may be flanked by inverted terminal repeat (ITR) nucleotide sequences, including AAV2 ITR sequences. The encoded human Pax4 may have the amino acid sequence of SEQ ID NO: 1 and may be encoded by a nucleic acid comprising a nucleotide sequence having at least 85% identity to the nucleotide sequence of SEQ ID NO: 2, which encodes a human Pax4, or a sequence reverse complement thereof. In some embodiments, the nucleotide sequence encoding Pax4 is SEQ ID NO: 2. The 5′ and 3′ ITR sequences may be wild type sequences, such as AAV2 ITRs, or may include a modified ITR sequence (as either the 5′ ITR or 3″ ITR) that results in a double-stranded “self complementary” AAV vector.

Particular expression cassettes are provided and have nucleotide sequences of SEQ ID NO: 10 (CAG-hPax4), SEQ ID NO: 11 (MGP-hPax4), or SEQ ID NO: 12 RIP-hPax4) (or a sequence reverse complementary thereto), or may be at least 85% identical to the nucleotide sequence of SEQ ID NO: 10 (CAG-hPax4), SEQ ID NO: 11 (MGP-hPax4), or SEQ ID NO: 12 RIP-hPax4), and encode a human Pax4.

Also provided are viral vectors (including AAV genomes) that comprise a Pax4 expression cassette flanked by a 5′ITR and a 3′ITR (including a modified ITR sequence for generating double stranded or self-complementary genomes). Included are genomes having the nucleotide sequence of SEQ ID NO: 13 (scAAV-CAG-hPax4), SEQ ID NO: 14 (scAAV-MGP-hPax4), or SEQ ID NO: 15 (scAAV-RIP-hPax4).

Also provided are plasmids comprising these expression cassettes, including the plasmid pscAAV-CAG-hPax4-KanR, depicted in FIG. 1, the nucleotide sequence of one strand of which is SEQ ID NO: 16; the plasmid pscAAV-MGP-hPax4-KanR, depicted in FIG. 2, the nucleotide sequence of one strand of which is SEQ ID NO: 17 and pscAAV-RIP-hPax4-KanR, depicted in FIG. 3, the nucleotide sequence of one strand of which is SEQ ID NO: 18. The nucleotide sequences depicted are one stranded of the double-stranded plasmid, the other strand being the reverse complement of the provided nucleotide sequence.

Also provided are recombinant AAV virions comprising a nucleic acid comprising an expression cassette described herein encoding hPax4 (for example, the expression cassettes of SEQ ID NO: 10, 11 or 12) or the viral genomes described herein (SEQ ID NO: 13, 14, or 15) and an AAV capsid. The AAV capsid may be an AAV6 capsid (having a capsid protein with an amino acid sequence of SEQ ID NO: 19) or an AAV8 capsid (having a capsid protein with an amino acid sequence of SEQ ID NO: 23) or an AAV9 capsid (having a capsid protein with an amino acid sequence of SEQ ID NO: 21).

Provided are methods of increasing insulin production in a subject in need thereof, methods of increasing the number of β cells in pancreatic islets of a subject in need thereof, methods of promoting transdifferentiation of α cells to β cells in a subject in need thereof, by administration of a composition comprising an effective amount of the AAV virions disclosed herein that express Pax4 in pancreatic cells. Also provided are methods of treating diabetes, including Type 1 diabetes (T1D) or type 2 diabetes (T2D) in a subject in need thereof and in preventing or reducing the risk of an individual at high risk of developing diabetes (for example, being pre-diabetic, being positive for auto-antibodies against pancreatic islet cells) from developing diabetes by administration of a composition comprising an effective amount of the AAV virions disclosed herein that express Pax4 in pancreatic cells. In some embodiments, the subject is a human. Provided are pharmaceutical compositions and methods of administration, including, but not limited to, delivery to the pancreas.

Also provided are host cells for and methods of producing the recombinant AAV virions as described herein.

Pax4 gene therapy has some unique advantages, which include: 1) the use of a single transcription factor Pax4, which makes gene delivery easier because there is no need to consider the order or balance of gene expression of multiple proteins; and 2) dual functionality of Pax4 in promoting α-to-β transdifferentiation and β cell survival, which work synergistically to improve β cell mass.

### Embodiments

Embodiment 1. A recombinant adeno-associated virus (AAV) vector comprising an expression cassette comprising a nucleotide sequence encoding hPax4, operably linked to a promoter that promotes expression of the nucleotide sequence encoding hPax4 in pancreatic islet cells and a polyadenylation (poly(A)) signal sequence; the promoter comprising a CAG promoter, a rodent or human glucagon promoter, or a rodent or human insulin promoter; wherein the expression cassette is flanked by inverted terminal repeat (ITR) nucleotide sequences.

Embodiment 2. The recombinant AAV vector of embodiment 1 in which the hPax4 comprises the amino acid sequence of SEQ ID NO: 1.

Embodiment 3. The recombinant AAV vector of embodiments 1 or 2 in which the nucleic acid that encodes hPax4 comprises a nucleotide sequence having at least 85% identity to the nucleotide sequence of SEQ ID NO: 2 or the reverse complement thereof.

Embodiment 4. The recombinant AAV vector of any one of embodiments 1 to 3 in which the nucleic acid that encodes hPax4 comprises or consists of the nucleotide sequence of SEQ ID NO: 2 or the reverse complement thereof.

Embodiment 5. The recombinant AAV vector of any one of embodiments 1 to 4 wherein the promoter has the nucleotide sequence of SEQ ID NO: 4 (CAG promoter), SEQ ID NO: 5 (Mouse Glucagon Promoter) or SEQ ID NO: 6 (Rat Insulin Promoter).

Embodiment 6. The recombinant AAV vector of any one of c embodiments 1 to 5 wherein the poly A signal sequence is an SV40 polyadenylation signal sequence having a nucleotide sequence of SEQ ID NO: 7, or the reverse complement thereof.

Embodiment 7. The recombinant AAV vector of any one of embodiments 1 to 6 which is a self-complementary vector.

Embodiment 8. The recombinant AAV vector of any one of embodiments 1 to 7 wherein the ITR nucleotide sequences comprise a 5′ ITR having a nucleotide sequence of SEQ ID NO: 8 and a 3′ ITR having a nucleotide sequence of SEQ ID NO: 25 or a modified 3′ ITR having a nucleotide sequence of SEQ ID NO: 9, or the reverse complement thereof, or a modified 5′ITR sequence and a 3′ ITR sequence of SEQ ID NO: 25, or the reverse complement thereof.

Embodiment 9. The recombinant AAV vector of any one of embodiments 1 to 8 in which the expression cassette has the nucleotide sequence of SEQ ID NO: 10 (CAT-hPax4), SEQ ID NO: 11 (MGP-hPax4), or SEQ ID NO: 12 (RIP-hPax4), or the reverse complement thereof.

Embodiment 10. The recombinant AAV vector of any one of embodiments 1 to 9 comprising the nucleotide sequence of SEQ ID NO: 13 (scAAV-CAG-hPax), SEQ ID NO: 14 (scAAV-MGP-Pax4) or SEQ ID NO: 15 (scAAV-RIP-hPax), or the reverse complement thereof.

Embodiment 11. A recombinant AAV virion comprising: 1) an AAV capsid comprising an AAV capsid protein and having a tropism for pancreatic islet cells; and 2) the recombinant AAV vector of any one of embodiments 1-10.

Embodiment 12. The AAV virion of embodiment 11, wherein the AAV capsid protein has an amino acid sequence at least 85% identical to an AAV6 capsid protein (SEQ ID NO: 19) or an AAV8 capsid protein (SEQ ID NO: 23) or an AAV9 capsid protein (SEQ ID NO: 21).

Embodiment 13. The AAV virion of embodiment 12, wherein the AAV capsid protein has the amino acid sequence of SEQ ID NO: 19 or 21 or 23.

Embodiment 14. A composition for use for increasing insulin production in a subject in need thereof, the composition comprising a therapeutically effective amount of the AAV virion of any one of embodiments 11 to 13 such that the nucleotide sequence encoding hPax4 is expressed in pancreatic islet cells when administered to the subject.

Embodiment 15. A composition for use for increasing the numbers of 3-cells in a subject in need thereof, the composition comprising a therapeutically effective amount of the AAV virion of any one of embodiments 11-13 such that the nucleotide sequence encoding hPax4 is expressed in pancreatic islet cells when administered to the subject.

Embodiment 16. A composition for use in a subject for treating, ameliorating or reducing the symptoms of diabetes mellitus in a subject suffering therefrom, the composition comprising a therapeutically effective amount of the AAV virion of any one of embodiments 11 to 13 such that the nucleotide sequence encoding hPax4 is expressed in pancreatic islet cells when administered to the subject.

Embodiment 17. The composition for use of embodiment 16, wherein the diabetes mellitus is Type 1 diabetes.

Embodiment 18. The composition for use of embodiment 16, wherein the diabetes mellitus is Type 2 diabetes.

Embodiment 19. A composition for use in reducing the risk of developing or slowing the development of diabetes in a subject positive for anti-islet autoantibodies, the composition comprising a therapeutically effective amount of the AAV virion of any one of embodiments 11 to 13 such that the nucleotide sequence encoding hPax4 is expressed in pancreatic islet cells when administered to the subject.

Embodiment 20. The composition for use in any one of embodiments 14 to 19, wherein said composition is administered by systemic administration, intravenous administration, intramuscular administration, intraperitoneal administration, or subcutaneous administration.

Embodiment 21. The composition for use in any one of embodiments 14 to 19, the wherein said composition is administered by direct administration to the pancreas, intraductal administration, intra-bile ductal (IBD) administration, or endoscopic retrograde cholangio-pancreatography (ERCP).

Embodiment 22. The composition for use of any one of embodiments 14 to 21, wherein the AAV virion of any one of embodiments 11 to 13 is formulated in a suitable carrier.

Embodiment 23. The composition for use of any one of embodiments 14 to 22, wherein at least one of fasting blood glucose levels, GTT levels, C peptide levels or A1C levels is reduced in the subject at least 1 month, 2 months, 3 months, 4 months, 5 months or 6 months after administration of the composition as compared to the level prior to the administration.

Embodiment 24. The composition for use of any one of embodiments 14 to 23, wherein at least one of fasting blood glucose levels, GTT levels, C peptide levels or A1C levels is at pre-diabetic levels in the subject at least 1 month, 2 months, 3 months, 4 months, 5 months or 6 months after administration of the composition.

Embodiment 25. The composition for use of any one of embodiments 14 to 24, wherein fasting blood glucose levels are 100 to 125 mg/dL, or even below 100 mg/dL, glucose tolerance test (GTT) levels are 140 to 199 mg/dL, C-peptide levels are between about 0.5 ng/mL to about 2.0 ng/mL, or A1C levels are between 5.7 and 6.5%.

Embodiment 26. The composition for use of any one of embodiments 14 to 25, wherein the subject is a human.

Embodiment 27. A method for increasing insulin production in a subject in need thereof, the method comprising administering a therapeutically effective amount of the AAV virion of any one of embodiments 11 to 13 such that the nucleotide sequence encoding hPax4 is expressed in pancreatic islet cells.

Embodiment 28. A method for increasing the numbers of β-cells in a subject in need thereof, the composition comprising a therapeutically effective amount of the AAV virion of any one of embodiments 11-13 such that the nucleotide sequence encoding hPax4 is expressed in pancreatic islet cells.

Embodiment 29. A method for treating, ameliorating or reducing the symptoms of diabetes mellitus in a subject suffering therefrom, said method comprising administering a therapeutically effective amount of the AAV virion of any one of embodiments 11 to 13 such that the nucleotide sequence encoding hPax4 is expressed in pancreatic islet cells.

Embodiment 30. The method of embodiment 29, wherein the diabetes mellitus is Type 1 diabetes.

Embodiment 31. The method of embodiment 29, wherein the diabetes mellitus is Type 2 diabetes.

Embodiment 32. A method for reducing the risk of developing or slowing the development of diabetes in a subject positive for anti-islet autoantibodies, the method comprising administering a therapeutically effective amount of the AAV virion of any one of embodiments 11 to 13 such that the nucleotide sequence encoding hPax4 is expressed in pancreatic islet cells.

Embodiment 33. The method of any one of embodiments 27 to 32, wherein said administering is by systemic administration, intravenous administration, intramuscular administration, intraperitoneal administration, or subcutaneous administration.

Embodiment 34. The method of any one of embodiments 27 to 32, wherein said administering is by direct administration to the pancreas, intraductal administration, intra-bile ductal (IBD) administration, or endoscopic retrograde cholangio-pancreatography (ERCP).

Embodiment 35. The method of any one of embodiments 27 to 34, wherein the AAV virion of any one of embodiments 11 to 13 is formulated in a suitable carrier.

Embodiment 36. The method of any one of embodiments 27 to 35, wherein at least one of fasting blood glucose levels, GTT levels, C peptide levels or A1C levels is reduced in the subject at least 1 month, 2 months, 3 months, 4 months, 5 months or 6 months after administration of the composition as compared to the level prior to the administration.

Embodiment 37. The method of any one of embodiments 27 to 36, wherein at least one of fasting blood glucose levels, GTT levels, C peptide levels or A1C levels is at pre-diabetic levels in the subject at least 1 month, 2 months, 3 months, 4 months, 5 months or 6 months after administration of the composition.

Embodiment 38. The method of any one of embodiments 27 to 37, wherein fasting blood glucose levels are 100 to 125 mg/dL, or even below 100 mg/dL, glucose tolerance test (GTT) levels are 140 to 199 mg/dL, C-peptide levels are between about 0.5 ng/mL to about 2.0 ng/mL, or A1C levels are between 5.7 and 6.5%.

Embodiment 39. The method of any one of embodiments 27 to 38, wherein the subject is a human.

Embodiment 40. An AAV vector plasmid comprising 1) an origin of replication and 2) the recombinant AAV vector of any one of embodiments 1 to 10.

Embodiment 41. The AAV vector plasmid of embodiment 40 comprising the nucleotide sequence of SEQ ID NO: 16, SEQ ID NO: 17, or SEQ ID NO: 18.

Embodiment 42. An cell for production of AAV virions, said cell comprising an AAV vector plasmid of embodiment 40 or 41, a second plasmid comprising nucleotide sequences encoding rep and cap; the cap encoding a VP1, a VP2, and a VP3; the rep encoding rep78, rep68, rep 52, and rep 40; and a third plasmid comprising nucleotide sequences encoding viral helper factors necessary for AAV virion product.

Embodiment 43. The cell of embodiment 42, the cap being AAV6 cap or AAV8 cap or AAV9 cap.

Embodiment 44. A method of producing an AAV virion, the method comprising culturing a host cell comprising the AAV vector plasmid of embodiment 40 or 41, a second plasmid encoding the cap and rep genes; the cap encoding the VP1, the VP2, and the VP3; the rep encoding rep78, rep68, rep 52, and rep 40; and a third plasmid encoding adenoviral helper factors, under conditions sufficient to produce the AAV virion.

Embodiment 45. The method of producing the AAV virion of embodiment 44, further comprising isolating the AAV virion.

### BRIEF DESCRIPTION OF THE DRAWINGS

FIG. 1 depicts a map of the plasmid pscAAV-CAG-hPax4-KanR (SEQ ID NO: 16), which comprises a 5′ AAV2 inverted-terminal repeat (ITR) (SEQ ID NO: 8) and a 3′ scITR (SEQ ID NO: 9) flanking an expression cassette comprising a CAG promoter (SEQ ID NO: 4), a nucleic acid encoding human Pax4 (hPax4) (SEQ ID NO: 2), and an SV40 poly(A) signal sequence (SEQ ID NO: 7). The plasmid further comprises an ampicillin resistance gene (AmpR) promoter controlling expression of a kanamycin resistance gene sequence (KanR), a CMV origin of replication (ori), and catabolite activator protein (CAP) binding site, a lactose (lac) promoter controlling a lac operator, and an M13 reverse primer region.

FIG. 2 depicts a map of the plasmid pscAAV-MGP-hPax4-KanR (SEQ ID NO: 17), which comprises a 5′ AAV2 inverted-terminal repeat (ITR) (SEQ ID NO: 8) and a 3′ scITR (SEQ ID NO: 9) flanking an expression cassette comprising a mouse glucagon promoter (SEQ ID NO: 5), a nucleic acid encoding human Pax4 (hPax4) (SEQ ID NO: 2), and an SV40 poly(A) signal sequence (SEQ ID NO: 7). The plasmid further comprises an ampicillin resistance gene (AmpR) promoter controlling expression of a kanamycin resistance gene sequence (KanR), a CMV origin of replication (ori), and catabolite activator protein (CAP) binding site, a lactose (lac) promoter controlling a lac operator, and an M13 reverse primer region.

FIG. 3 depicts a map of the plasmid pscAAV-RIP-hPax4-KanR (SEQ ID NO: 18), which comprises a 5′ AAV2 inverted-terminal repeat (ITR) (SEQ ID NO: 8) and a 3′ scITR (SEQ ID NO: 9) flanking an expression cassette comprising a rat insulin promoter (SEQ ID NO: 6), a nucleic acid encoding human Pax4 (hPax4) (SEQ ID NO: 2), and an SV40 poly(A) signal sequence (SEQ ID NO: 7). The plasmid further comprises an ampicillin resistance gene (AmpR) promoter controlling expression of a kanamycin resistance gene sequence (KanR), a CMV origin of replication (ori), and catabolite activator protein (CAP) binding site, a lactose (lac) promoter controlling a lac operator, and an M13 reverse primer region.

FIG. 4 is a graph showing the body weight (grams) of the STZ treated mouse model, and normal (wild-type) controls not treated with STZ or vector, over the days post-administration in each group with AAV vectors or PBS control, as indicated in the legend.

FIG. 5. Non-fasting blood glucose (BG) in mg/dL. Mean BG of all STZ treated mice in each treatment group (administered a vector or control PBS as indicated in the legend) and normal controls not treated with STZ or vector, over days after vector or PBS control administration.

FIG. 6. Non-fasting BG (mg/dL) of only the STZ treated mice administered with vectors or PBS control one week after last STZ dose as indicated in the legend (i.e., the slower T1D developing STZ mice) compared to normal mice not treated with STZ or vector. Mean BG of these mice throughout the study duration. AAV9.CAG-Pax4 and AAV9.MGP-Pax4-treated mice showed significantly lower non-fasting BG than the control AAV9.CAG-GFP-treated mice (p=0.0085 and p=0.0327, respectively, as assessed by 2-way ANOVA). AAV9.CAG-Pax4 also showed significantly lower BG than the PBS control group (p=0.0259).

FIGS. 7A and 7B. Glucose Tolerance Test (GTT) (mg/dL blood glucose) of STZ treated mice administered with vectors and control PBS one week after last STZ dose compared to normal mice not treated with STZ or vector. Following overnight fasting, the mice were injected with 1 g/Kg bodyweight of glucose, and their BG was measured prior to glucose injection and 15, 30, 45, 60, 90 and 120 minutes after glucose injection. (A) GTT data. (B) area under the curve (AUC) analysis of the GTT data. The data are expressed as Mean±SEM. *: p<0.05; **: p<0.01.

FIGS. 8A and 8B. Glucose-Stimulated Insulin Secretion (GSIS) assay for mice treated with test articles and controls one week after last STZ dose. The data are expressed as Mean±SEM. Panels A and B are the same data presented as bar or line graph, respectively.

FIGS. 9A-9C. Circulating hormone levels in all STZ treated mice and normal controls were analyzed at the end of animal experiments (3 months after vector administration). A. Insulin (ng/ml); B. Glucagon (ng/ml); C. C-peptide (pM).

FIGS. 10A-10C. Circulating hormone levels were analyzed at the end of the animal experiment in only those STZ treated mice administered vector or PBS control one week after the last STZ dose (i.e., the slower T1D developing STZ mice) and normal untreated controls. A. Insulin; B. Glucagon; C. C-peptide. *:p<0.05; **:p<0.01.

### DETAILED DESCRIPTION

### Definitions

When interpreting the description and claims, “comprising” “consisting of” and “consisting essentially of” should be given their ordinary meanings. For purposes of the disclosure herein, the term “comprising” contemplates and provides support for embodiments which replace the term “comprising” with the term “consisting of” and/or the term “consisting essentially of” For example, when “comprising A, B, or C” is recited, contemplated therein is replacing such, and provides support for embodiments which replace, with: “is A, B, or C,” “consists of A, B, and C,” “consists essentially of A, B, or C,” or any equivalent thereof.

Further, recitation of “or” contemplates and supports, “one or more of,” “one or a combination of,” or “and/or.” For example, “A, B, or C” contemplates and supports embodiments with: A alone; B alone; C alone; the combination of A and B; the combination of A and C; the combination of B and C; and the combination of A, B, and C. Further to which, within recitation of “closed” language (e.g. consisting of), as well as within recitation of “open” language (e.g. comprising), the recitation of a list, as in “A, B, or C,” contemplates one or a combination within that list, unless otherwise specified. For example, “consisting of A, B, or C” contemplates and supports embodiments with: A alone; B alone; C alone; the combination of A and B; the combination of A and C; the combination of B and C; and the combination of A, B, and C. Recitation of “and/or” contemplates and supports not only the combination of all within the list (i.e. “A, B, and/or C” contemplates “A, B, and C”), but also “one or more of” or “one or a combination of” For example “A, B, and C” contemplates: A alone; B alone; C alone; the combination of A, B and C; the combination of A and B; the combination of A and C; and the combination of B and C.

The recitation of a list of alternatives with an “and,” as in for example “selected from the group consisting of,” contemplates and provides support for combinations within that list, unless otherwise stated. For example, “is selected from the group consisting of A, B, and C” is to be understood to contemplate and support “is selected from the group consisting of A, B, C, and combinations thereof” and to be coextensive with “is at least one selected from the group consisting of A, B, and C” or such that “group” includes: A alone, B alone, C alone, A and B in combination, A and C in combination, B and C in combination, and A, B, and C in combination.

Further, recitation of a component in an embodiment also contemplates and supports exclusion, explicitly, of said component from the embodiment. For example, “comprising A, B, or C” supports embodiments, which comprise A or B, but specifically exclude C.

The articles “a” and “an” are used herein to refer to one or to more than one (i.e., to at least one) of the grammatical object of the article. By way of example, “an element” means one element or more than one element. For example, “comprising an A, a B, or a C” contemplates and supports embodiments comprising two or more A, two or more B, and two or more C.

Unless defined otherwise, all technical and scientific terms have the same meaning as commonly understood by one of ordinary skill in the art to which the embodiments pertain. The preferred materials and methods are described, but it is understood that any methods and materials similar or equivalent to those described can be used in the practice of embodiments. The terminology used herein is for the purpose of describing particular embodiments only and is not intended to be limiting. In describing and claiming the present invention, the following terminology will be used.

“About” as used herein when referring to a measurable value such as an amount, a temporal duration, and the like, is meant to encompass variations of +20% or ±10%, more preferably ±5%, even more preferably +1%, and still more preferably +0.1% from the specified value, as such variations are appropriate to perform the embodiments.

As used herein, the term “cell line” refers to a population of cells capable of continuous or prolonged growth and division in vitro. Often, cell lines are clonal populations derived from a single progenitor cell. Spontaneous or induced changes can occur in karyotype during storage or transfer of such clonal populations. Therefore, cells derived from the cell line referred to may not be precisely identical to the ancestral cells or cultures, and the cell line referred to includes such variants.

The term “expression” as used herein is defined as the transcription and/or translation of a particular nucleotide sequence driven by its promoter.

As used herein, the term “host cell” refers to any cell that harbors, or is capable of harboring, a substance of interest. Often a host cell can be a mammalian cell (e.g., a non-human primate, rodent, or human cell). In some aspects, the host cell can be a mammalian cell, a yeast cell, a bacterial cell, an insect cell, a plant cell, or a fungal cell. A host cell can be used as a recipient of an AAV helper construct, an AAV plasmid encoding a recombinant AAV genome comprising a transgene, an accessory function vector, or other transfer DNA associated with the production of recombinant AAV (rAAV) virions. The term includes the progeny of the original cell which has been transfected. Thus, a “host cell” as used herein can refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

“Identity” as used herein refers to the subunit sequence identity between two polymeric molecules particularly between two amino acid molecules, such as, between two polypeptide molecules or two nucleic acid molecule, such as polynucleotides. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical. In the case of an insertion or deletion, identity is understood to realign those thereafter which would be identical and is considered to be not identical at the insertion or deletion.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

A “nucleic acid,” as used herein, is interchangeable with “polynucleotide” or “a specific sequence of nucleotide.” These terms refer to a discrete sequence that performs a specific function directly or indirectly in a cell. That function includes encoding a sequence of a gene that is transcribed into mRNA and translated into protein and regulating said transcription (i.e. as a promoter would) and/or translation (i.e. as microRNA would). A nucleic acid inherently has a sequence. Thereby, “a nucleic acid comprising SEQ ID NO: X” can be used to contemplate and support “a nucleic acid comprising the sequence of SEQ ID NO: X.” In recombinant molecular biology, discrete nucleic acids can be combined. In some embodiments, a nucleic acid that encodes a protein can be ligated to a promoter (which is a nucleic acid), and a cis-acting element of a viral vector (i.e. an inverted-terminal repeat (ITR), which is also a nucleic acid). For convenience, a “nucleic acid” might be used to refer to the discrete elements within the larger nucleic acid, which could be referred to as “a polynucleotide,” “an expression region” (i.e. a polynucleotide comprising a promoter and a nucleic acid that encodes a protein), or “a vector” (see definition below).

“Encoding” refers to the inherent property of a nucleic acid to serve as a template, whether directly (i.e. a sense strand) or indirectly (i.e. an antisense strand) for synthesis of peptide, polypeptides, proteins, or other nucleic acids (i.e. rRNA, tRNA, microRNA). A nucleic acid can “encode” whether it is the sense strand, antisense strand, or a double-stranded segment thereof. The sense strand directly encodes the rRNA, tRNA, microRNA, or mRNA. The mRNA then serves as the template for translation of a peptide, polypeptide, or protein. The anti-sense strand is generally considered to be the reverse complementary sequence and is sometimes called a “non-coding”strand in the art (although for present purposes “non-coding” is a misnomer because the non-coding strand still “encodes” the genetic information by perpetuating it during semi-conservative replication by acting as a template for the polymerization of a new, sense strand). Within semi-conservative replication two single strands in double-stranded nucleic acids are separated, and a new strand is polymerized from the information from each of the single-stranded nucleic acids (i.e. single-stranded template), regardless of whether one single-stranded template is the sense strand (e.g. that which is used to transcribe mRNA and thereby, or directly, encode the translate or protein) or the antisense strand. By perpetuating the genetic information, the antisense strand is still encoding the genetic information for, for example, a protein. Accordingly, “a nucleic acid encoding X”, includes sense and antisense sequences or strands whether X is a peptide, a polypeptide, or a protein or X is a sequence that encodes a rRNA, tRNA, microRNA, antisense RNA, etc.

Further to which, “nucleic acid encoding X,” includes RNA, DNA, and combinations thereof, since nucleic acids are synthesized from transcription, reverse-transcription, and replication, as naturally occurring processes and man-made processes (recombinant biology, molecular biology, etc.).

Accordingly, a recited nucleic acid sequence contemplates and supports the complementary version thereof, the reverse complementary version thereof, and double-stranded versions thereof. That is, “a nucleic acid comprising SEQ ID NO: X” is to be understood, contemplate, and support “a nucleic acid comprising the reverse complementary of SEQ ID NO: X” or, using the nomenclature regarding the prime symbol as in “′”, “a nucleic acid comprising SEQ ID NO: X′,” unless otherwise specified. For example, “the nucleic acid comprising SEQ ID NO: X” wherein SEQ ID NO: X is 5′-ATGCC-3′ contemplates and supports the reverse complement of SEQ ID NO: X, and specifically 5′-GGCAT-3.

As noted above, a recited nucleic acid sequence contemplates and supports conversion between RNA and DNA versions thereof. For example, if SEQ ID NO: X is “5′-ATGCC-3′,” contemplated and supported is 5′-AUGCC-3′, as well as the reverse complementary thereof, 5′-GGCAU-3.

With regard to an AAV vector or an AAV virion, the above-noted incorporation of reverse complementary sequences and double-stranded segments into the definition of “a nucleic acid” and the above-noted use of “encoding” as including sense and antisense strands, is intended to incorporate the means by which the AAV vector can introduce an exogenous nucleic acid sequence that encodes nucleic acid or a protein into the cell. It is further intended to incorporate, in some embodiments, processes whereby said introduction results in the expression of said nucleic acid (i.e. miRNA or antisense RNA) or protein (i.e., Pax4).

Take for example, a nucleic acid encoding a protein, and an AAV vector comprising a nucleic acid encoding said protein. When a typical AAV vector comprising one sense or one antisense strand of the nucleic acid that encodes said protein enters the cell, the inverted-terminal repeats (ITRs) prime the synthesis of a sequence reverse complementary to the sense strand or antisense strand of the nucleic acid that encodes said protein. The polymerization thereby forms a segment of double-stranded DNA comprising the sense and antisense strands, regardless of whether the sense version or antisense version was first introduced to the cell. In this regard, the entire nucleic acid including ITRs and sense and antisense nucleic acids encoding a protein can be one single-stranded DNA, which loops upon itself to form a double-stranded segment, wherein the base-pairs the sense and antisense nucleic acids encoding the protein align.

From this segment of double-stranded DNA, transcription of mRNA and translation of said protein is achieved from said sense strand of DNA, regardless of whether the AAV vector comprised only the sense strand or only the antisense strand when first entering the cell. In this regard, “an AAV vector comprising a nucleic acid encoding protein X” includes, contemplates, and supports embodiments in which the nucleic acid is the sense strand encoding protein X, the antisense strand encoding protein X, a double-stranded nucleic acid encoding protein X, and a single stranded nucleic acid comprising sense and antisense strands wherein the sense and antisense strands form a segment of double-stranded nucleic acid.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence.

The term “promoter” as used herein is defined as a DNA sequence recognized by the synthetic machinery of the cell, or introduced synthetic machinery, required to initiate the specific transcription of a polynucleotide sequence. In some instances, this sequence may be the core promoter and in other instances, this sequence may also include, or be an enhancer alone and/or other regulatory elements which are required for expression of the gene product.

In certain instances the promoter may comprise enhancer elements, exons, and introns from one or a variety of viruses and animals, and thereby the term “promoter” shall be understood to not be limited to being a non-expressed sequence, nor exclude a non-expressed sequence that is between expressed sequences (i.e. introns), nor be limited to exclude an enhancer alone so long as the combination of sequences used to construct the promoter are capable of initiating the specific transcription of a polynucleotide sequence.

A “constitutive” promoter is a nucleotide sequence which, when operably linked with a polynucleotide that encodes or specifies a gene product, causes the gene product to be produced in a cell under most or all physiological conditions of the cell and without requiring the addition of exogenous factors or the introduction of a different phenotype to the cell. This constitutive promoter can be cell-specific so long as it is produced in the specific, or target, cell under most or all physiological conditions of the cell. The CAG promoter is an example of a constitutive promoter in a broad range of target cell types.

As used herein, the term “recombinant cell” refers to a cell into which an exogenous DNA segment, such as DNA segment that leads to the transcription of a biologically-active polypeptide or production of a biologically active nucleic acid such as an RNA, has been introduced.

A “target gene” refers to a nucleic acid encoding a target protein to be expressed within a target cell upon entry of the vector carrying the target gene into the cell. The target gene includes naturally occurring polymorphisms (i.e. variants) and man-made modifications to the wild-type gene so long as the target protein is still expressed. An example of such man-made modifications includes codon-optimization.

A “target protein” refers to a man-made or naturally occurring protein of interest to be introduced by vector into a host cell. One some embodiments, the target protein, as encoded in the genome of the host cell, is not functional because of a polymorphism in the gene sequence resulting in some mistranscription, missense, or mistranslation of the gene whereby reduced or no target protein or inoperable target protein is produced (i.e. a polymorphism results in an early stop codon) or an attenuation in the activity of the target protein, as encoded by and expressed from the genome of a subject.

In some embodiments, the target protein comprises mammalian Pax4, including, in an embodiment, human Pax4. It is to be understood and contemplated that “Pax4” encompasses naturally-occurring versions (i.e. human Pax4 and other mammalian Pax4, such as mouse or rat Pax4) and non-naturally occurring Pax4 (i.e. amino-acid additions, deletions, or substitution of Pax4, which increase or decrease the activity compared to that of naturally occurring Pax4) so long as the protein referred to as Pax4 has at least the activity to, when expressed in an a cell, transdifferentiate that cell to a β cell. In some embodiments, the non-naturally occurring Pax4 has at least, or no more than, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% of the activity of the corresponding naturally-occurring Pax4, wherein “corresponding” contemplates and provides support for that to which the additions, deletions, or substitutions were applied. In some embodiments, the Pax4 is a human Pax4 and, in some embodiments, has the amino acid sequence of SEQ ID NO: 1. In alternate embodiments, the Pax4 has an amino acid sequence that has at least 99%, 95%, 90%, 85% or 80% sequence identity to SEQ ID NO: 1 and has Pax4 activity. In other embodiments, the Pax4 is encoded by the nucleotide sequence of SEQ ID NO: 2, or a nucleotide sequence that is at least 99%, 95%, 90%, 85% or 80% identical to SEQ ID NO: 2 and encodes hPAX4 having the amino acid sequence of SEQ ID NO: 1 or an hPAX4 which has an amino acid sequence that has at least 99%, 95%, 90%, 85% or 80% sequence identity to SEQ ID NO: 1 and has PAX4 activity.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter. Mammalian insulin and glucagon promoters are examples of tissue-specific promoters for expression in pancreatic islet cells.

A “vector” is a nucleic acid capable of delivering a target gene to the interior of a cell, and includes not only the expression-region (i.e. a promoter and a nucleic acid encoding a protein or even a nucleic acid), but also some cis-acting genetic component. The cis-acting genetic component provides for packaging within a virion, expression in a cell, replication in a cell, or a combination thereof.

By way of example, nucleic acids comprising inverted-terminal repeats (ITRs) from adeno-associated viruses (AAVs) constitute a vector when adjoined to the nucleic acid encoding a target protein because the ITRs will provide for the nucleic acid encoding the target protein to be packaged within an AAV virion. ITRs may also provide other cis-acting functions for expression of the nucleic acid encoding the target protein in the host cell upon entry of the vector into the host cell. Such cis-acting functions of ITRs include aiding in concatemer formation for genomic insertion; initiation of second strand formation in the case of a single-stranded (ss) AAV (ssAAV) vector; or initiation of replication and transcription in the case of ssAAV and self-complementary (sc) AAV (scAAV) vectors. In this regard, the AAV ITRs can be characterized based on the nucleic acid sequences providing such cis-acting functions from the serotypes of AAVs. That is, an ITR isolated from an AAV2 serotype can be known as an AAV2 ITR, even though the ITR generally does not contribute to the serotype of an AAV.

Further it is understood that although the scAAV ITR (e.g. SEQ ID NO: 9) was developed by mutating or altering a wild type AAV2 ITR (e.g. SEQ ID NO: 8, which is the 5′ flanking ITR) and specifically the terminal resolution site (trs) in the D-sequence, which is responsible for signaling for packaging (a packaging sequence), the scAAV ITR provides for a function not provided for by the wild-type AAV ITR, which is the generation of an AAV vector comprising an expressing region and a reverse complement thereof prior to packaging within the AAV virion, generating a scAAV. In some embodiments, the scAAV vector is then packaged into a recombinant scAAV virion.

The scAAV vector may exhibit at least, or no more than, 50%, 60%, 70%, 80%, 90%, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 110-fold, 120-fold, 130-fold, or 140-fold more efficient transduction than a corresponding vector comprising a wild-type AAV2 ITR and no complement of the expressing region. Without wishing to be bound to a particular theory, it is believed that the host-cell synthesis of a double-stranded segment in a ssAAV vector is rate limiting in ssAAV vector transduction, and that by providing an expressing region and the reverse complement thereof said scAAV ITR (and vector comprising said scAAV ITR) provides for the above-noted increase in efficiency of transduction.

By way of example, a plasmid can comprise an origin of replication (e.g., on from cytomegalovirus) which allows for the replication of the target gene within a cell, and such a plasmid is thereby a vector.

“Expression vector” refers to a vector comprising an expressing region. An expressing region includes a recombinant polynucleotide comprising a nucleic acid that controls expression (i.e. a promoter) and a nucleic acid that encodes. The nucleic acid that encodes includes a nucleic acid that encodes a protein. Generally, the promoter is operatively linked to the nucleic acid that encodes the target protein in a manner that is capable of promoting expression of the protein upon entry of the vector into the host cell. In some embodiments, the promoter can be operably linked by ensuring that there is not codon misalignment.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The nucleotide and amino acid sequences provided herein are set out in Table 1.

### AAV Vectors

In some embodiments, a recombinant adeno-associated virus (AAV) vector is provided, which comprises an expressing region or expression cassette and at least two inverted-terminal repeats (ITRs) flanking the expression cassette; the expression cassette comprises a promoter and a nucleic acid that encodes Pax4, including human Pax4; the promoter is operably linked to the expression of the Pax4-encoding nucleic acid, and a polyadenylation signal further operably linked to the Pax4 coding sequence; and the at least two ITRs flank the expressing region or expression cassette. The expression cassette may further include other regulatory sequences such as Kozak sequences, enhancers, intron sequences and WPRE sequences.

In some embodiments, the Pax4, and in some embodiments, human Pax4, encoded by the vector comprises an amino acid sequence comprising at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; the amino acid sequence of SEQ ID NO: 1, while retaining Pax4 activity. In some embodiments, the Pax4 comprises or consists of the amino acid sequence of SEQ ID NO: 1. In some embodiments, the nucleic acid that encodes Pax4 comprises a nucleic acid having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; SEQ ID NO: 2 or a reverse complementary sequence thereto and encodes a human Pax4 having an amino acid sequence of SEQ ID NO: 1 or a variant thereof that retains Pax4 activity. In some embodiments, the nucleic acid that encodes Pax4 comprises or consists of SEQ ID NO: 2 or a reverse complementary sequence thereto. In some embodiments, the nucleic acid has at least 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to SEQ ID NO:2 and encode the human Pax4 protein having an amino acid sequence of SEQ ID NO: 1, or a variant thereof having human Pax4 activity. In some embodiments, a Kozak sequence is introduced just 5′ to the Pax4 coding sequence to promote translation of the Pax4 protein from the expression cassette. The Kozak sequence may be 5′GCCACC3′, SEQ ID NO: 3.

In some embodiments, the ITR, or at least two ITRs, comprises an AAV1 ITR, AAV2 ITR, AAV3 ITR, AAV4 ITR, AAV5 ITR, AAV6 ITR, AAV7 ITR, AAV8 ITR, or an AAV9 ITR, and in some embodiments may be an scAAV ITR. In some embodiments, the ITR, or at least two ITRs, comprises a AAV2 ITR or a scAAV ITR. In some embodiments, the ITR, or at least two ITRs, comprises AAV2 ITR and a scAAV ITR, where the 5′ ITR is the AAV2 ITR and the 3′ ITR is the scAAV ITR, or, alternatively, the 5′ ITR is the scAAV ITR and the 3′ ITR is the AAV2 ITR. In some embodiments, the scAAV ITR is an AAV2 ITR lacking at least one functional terminal resolution site (trs) in the D-sequence. In some embodiments, the scAAV ITR lacks at least one functional trs. In some embodiments, the scAAV ITR has at least one substitution, addition, or deletion in the at least one trs, wherein the at least one substitution, addition, or deletion confers lack of function to the at least one trs. In some embodiments, the AAV ITR has at least one a D-sequence deleted. In some embodiments, the D-sequence deleted is at the 3′ end of the ITR (ssD[−]). In some embodiments, the D-sequence deleted is a the 5′ end of the ITR (ssD[+]). In some embodiments, the ssD[−] sequence has at least one substitution, deletion, or addition that prevents binding of the 52-kDa-FK506-binding protein (FKBP52). In some embodiments, the AAV ITR has a D-sequence replaced with a transcription factor binding site. In some embodiments, the transcription factor binding site comprises an S-sequence. In some embodiments, the S-sequence comprises a Foxd3 binding site or a NF-pE1 binding site. In some embodiments, the transcription factor binding site or the S-sequence comprises a GATA-1 and GATA-2 binding site. In some embodiments, the ITR, or at least two ITRs, comprise a nucleic acid having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; SEQ ID NO: 8, or a reverse complementary sequence thereto, including SEQ ID NO: 9. In some embodiments, the ITR, or at least two ITRs, comprise a nucleic acid having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; SEQ ID NO: 8, or a reverse complementary sequence thereto, including SEQ ID NO: 25. In some embodiments, the ITR, or at least two ITRs, comprise SEQ ID NO: 8 (5′ ITR), and/or SEQ ID NO: 25 (3′ ITR), or a reverse complementary sequence thereto. In some embodiments, the ITR, or at least two ITRs, comprise SEQ ID NO: 8 (5′ ITR), and/or SEQ ID NO: 9 (3′ scITR), or a reverse complementary sequence thereto. In the case of a self-complementary vectors, one of the ITRs has a mutant sequence, for example, the 3′ ITR has the mutant sequence of SEQ ID NO: 9 and the ITR at the 5′ end of the expression cassette is an unmodified ITR, for example, and AAV2 ITR having a nucleotide sequence of SEQ ID NO: 8.

In some embodiments, the sequence encoding Pax4 is operably linked to a promoter, including a constitutive promoter. In some embodiments the promoter is a CAG promoter. A CAG promoter is a composite, synthetic promoter which contains the CMV early enhancer element, the chicken β-actin promoter and the first exon and first intron of the chicken β-actin gene and the splice acceptor of the rabbit β globin gene. See, e.g., Miyazaki et al, Gene 79:269-277 (1989) and Niwa et al, Gene 108:193-199 (1991). In some embodiments, the CAG “promoter” has a nucleotide sequence of SEQ ID NO: 4, or may be an at least 200, 300, 400, 500 or 600 nucleotide fragment thereof with promoter activity that promotes expression of the target gene in the appropriate tissues (or a reverse complement thereof as appropriate). In some embodiments, the promoter comprises a nucleic acid sequence having at least, or no more than, 85%, 86%, 87%, 88%, 89%, 90%, 91%, 92%, 93%, 94%, 95%, 96%, 97%, 98%, 99.0%, 99.100, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within SEQ ID NO: 4, or a reverse complementary sequence thereto and promotes Pax4 gene expression in the appropriate tissues. In some other embodiments, the promoter comprises or consists of a Rous sarcoma virus (RSV) LTR promoter, a cytomegalovirus (CMV) promoter, a simian virus (SV40) promoter, a dihydrofolate reductase promoter, a β-actin promoter, a phosphoglycerol kinase (PGK) promoter, a P5 promoter, a Ubc promoter, a tetracycline response element promoter, a UAS promoter, an Ac5 promoter, a polyhedrin promoter, a calmodulin-dependent protein kinase II-α (CaMKIIα) promoter, a Pax4 promoter, an H1 promoter, a U6 promoter, or an Alpha-1-antitrypsin promoter. In some embodiments, the β-actin promoter is a chicken β-actin (“CBA”) promoter or a human β-actin promoter.

In some embodiments, the Pax4 coding sequence is operably linked to a promoter that is tissue specific for pancreatic islet cells, including promoters specific for alpha cells or for beta cells. Such promoters include human or rodent (such as mouse or rat) insulin promoters or human or rodent (such as mouse or rat) glucagon promoters. In some embodiments, the promoter is mouse glucagon promoter. In an embodiment, the promoter consists of or comprises the nucleotide sequence of SEQ ID NO: 5, or may be an at least 200, 300, or 400 nucleotide fragment thereof with promoter activity, or a reverse complement thereof as appropriate, that promotes expression of the target gene in the appropriate tissues (such as pancreatic islet cells, including alpha and/or beta cells). In some embodiments, the promoter comprises a nucleic acid sequence having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within SEQ ID NO: 5, or a reverse complementary sequence thereto and promotes Pax4 gene expression in the appropriate tissues, such as pancreatic islet cells. In some embodiments, the promoter is a rat insulin promoter, see, Ullrich et al. “Rat insulin genes: construction of plasmids containing the coding sequences,” Science, 196 (4296): 1313-1319 (1977) (database Sequence ID: J00747.1 (BLAST)) and Dandoy-Dron et al. “Tissue-specific expression of the rat insulin 1 gene in vivo requires both the enhancer and promoter regions.” Differentiation. 58(4):291-5 (1995)).

In an embodiment, the promoter consists of or comprises the nucleotide sequence of SEQ ID NO: 6, or may be an at least 200, 300, or 400 nucleotide fragment thereof with promoter activity that promotes expression of the target gene in the appropriate tissues (such as pancreatic islet cells, or a reverse complement thereof as appropriate). In some embodiments, the promoter comprises a nucleic acid sequence having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within SEQ ID NO: 6, or a reverse complementary sequence thereto and promotes Pax4 gene expression in the appropriate tissues, such as pancreatic islet cells (including alpha and/or beta cells).

In some embodiments, the expressing region is in an anti-sense (e.g. reverse complementary) orientation or in sense orientation. In some embodiments, the vector comprises two or more expressing regions. In some embodiments, the two or more expressing regions comprises one in antisense orientation and another in sense orientation (e.g., as an scAAV).

In some embodiments, the expressing region further comprises a nucleic acid that encodes a polyadenylation (poly(A)) signal 3′ of the target gene coding sequence such that the expressed mRNA has a polyA tail. In some embodiments, the nucleic acid that encodes the poly(A) signal comprises a bovine growth hormone (bGH) poly(A) tail signal or a simian virus 40 (SV40) poly(A) tail signal. In some embodiments, the polyA signal has a nucleotide sequence of SEQ ID NO: 7 (SV40 polyA signal) (or a reverse complement thereof) or the polyA signal has at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; SEQ ID NO: 7 or a reverse complementary sequence thereto, which causes polyadenylation of the transcribed sequence.

In some embodiments, the recombinant AAV vector may be packaged into a recombinant AAV virion.

Provided, thus, are expression cassettes that can be incorporated into an AAV vector or plasmid for use in generating rAAV particles for gene replacement expression of the target gene. The expression cassettes are flanked by AAV ITRs. In particular embodiments, the expression cassette may have elements arranged as follows: 5′AAV2ITR-CAG promoter sequence-Kozak sequence-hPax4 coding sequence-SV40 polyA signal sequence-3′scAAV2 ITR. In some embodiments, the expression cassette has a nucleotide sequence of SEQ ID NO: 10 (not including the flanking ITR sequences) or 13 (including the flanking ITR sequences). In other embodiments, the gene expression cassette has elements arranged as follows: 5′AAV2ITR-MGP promoter sequence-Kozak sequence-hPax4 coding sequence-SV40 polyA signal sequence-3′scAAV2 ITR. In some embodiments, the expression cassette has a nucleotide sequence of SEQ ID NO: 11 (not including the flanking ITR sequences) or 14 (including the flanking ITR sequences. In particular embodiments, the expression cassette may have elements arranged as follows: 5′AAV2ITR-RIP promoter sequence-Kozak sequence-hPax4 coding sequence-SV40 polyA signal sequence-3′scAAV2 ITR. In some embodiments, the expression cassette has a nucleotide sequence of SEQ ID NO: 12 (not including the flanking ITR sequences) or 15 (including the flanking ITR sequences).

In some embodiments, the scAAV vector is then packaged into a recombinant AAV virion.

In some embodiments, the expressing region comprises a nucleic acid having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; SEQ ID NO: 10, SEQ ID NO: 11, SEQ ID NO: 12 (or SEQ ID NO: 13, 14 or 15 with flanking ITRs), or a reverse complementary sequence thereto, and is an expression cassette that expresses hPax4 in appropriate human tissues. In some embodiments, the expressing region or expression cassette comprises SEQ ID NO: 10, SEQ ID NO: 11, SEQ ID NO: 12 (or SEQ ID NO: 13, 14 or 15 with flanking ITRs), or sequences reverse complementary thereto.

### AAV Vector Plasmids

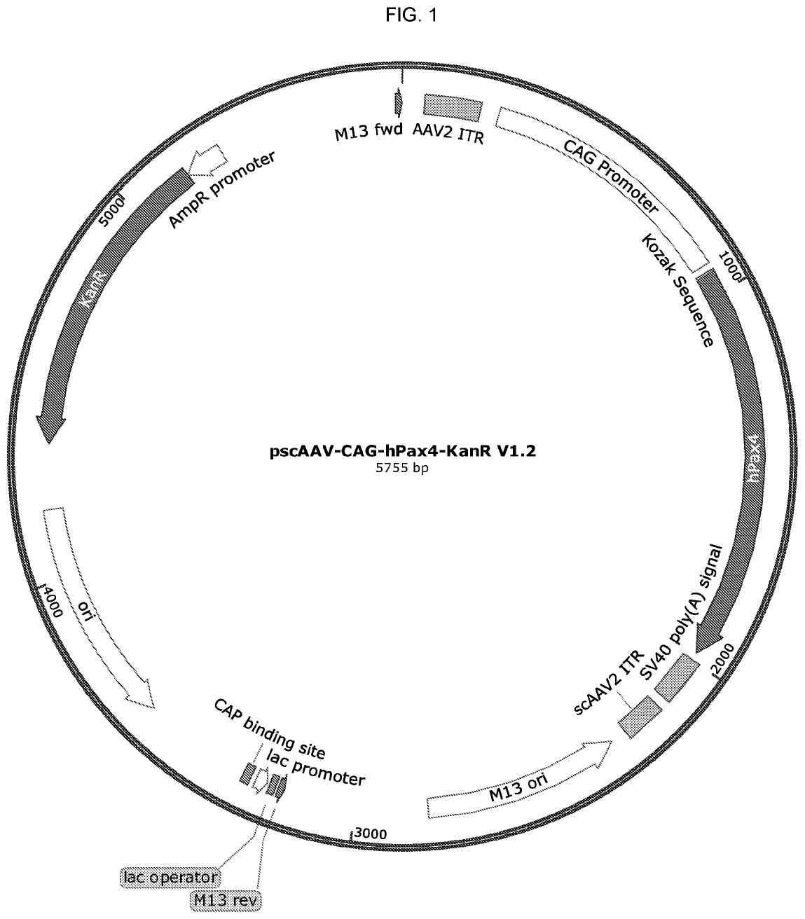

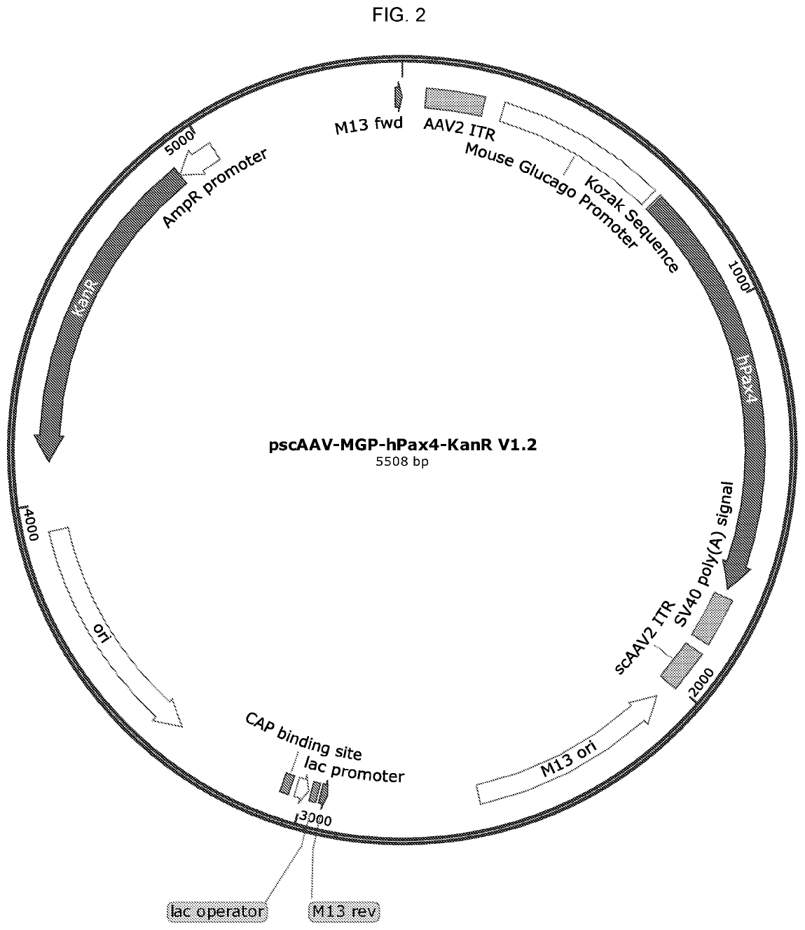

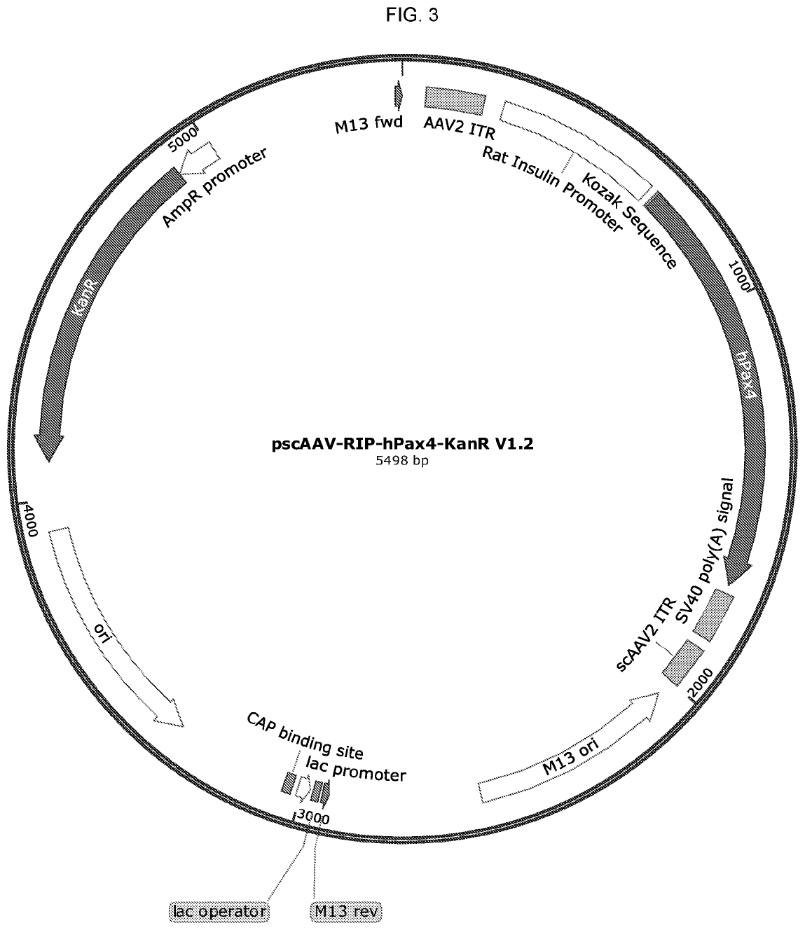

In some embodiments, an AAV vector plasmid is provided that may be used to prepare a recombinant AAV viral particle having a recombinant genome comprising a nucleotide sequence encoding the target gene operably linked to regulatory elements that promote expression in appropriate tissues. The plasmids provided herein generally have an origin of replication and selectable markers to permit reproduction of the plasmid and use in host cells for generating the recombinant AAV viral particles described herein. Exemplary plasmids are depicted in FIGS. 1-3. The plasmids provided herein include plasmids comprising the expression cassettes described herein. In particular, the AAV vector plasmids may comprise an expression cassette having a nucleotide sequence of SEQ ID NO: 10, 11 or 12. In some embodiments, the AAV vector plasmid comprises a nucleic acid having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; SEQ ID NO: 10, SEQ ID NO: 11, or SEQ ID NO: 12. Provided plasmids include pscAAV-CAG-hPax4-KanR (FIG. 1, SEQ ID NO: 16), pscAAV-MGP-hPax4, KanR (FIG. 2, SEQ ID NO: 17), and pscAAV-RIP-hPax4-KanR (FIG. 3, SEQ ID NO: 18).

In some embodiments, the AAV vector plasmid further comprises a bacterial expressing region. In some embodiments, the bacterial expressing region comprises a bacterial promoter and a nucleic acid that encodes a bacterial selecting region. In some embodiments, the nucleic acid that encodes the bacterial selecting region is operably linked to the bacterial promoter. In some embodiments, the nucleic acid that encodes the bacterial selecting region comprises a nucleic acid that encodes an antibiotic resistance gene or protein. In some embodiments, the antibiotic resistance gene or protein comprises an ampicillin resistance gene (AmpR) or a kanamycin resistance gene sequence (KanR). In some embodiments the bacteria promoter comprises AmpR promoter or a KanR promoter. In some embodiments, the AAV vector plasmid further comprises an origin of replication. In some embodiments, the origin of replication comprises a CMV origin of replication (ori). In some embodiments, the AAV vector plasmid further comprises a eukaryotic expressing region. In some embodiments, the eukaryotic expressing region comprises a eukaryotic promoter and a nucleic acid that encodes a eukaryotic selecting region. In some embodiments, the nucleic acid that encodes a eukaryotic selecting region is operably linked to the eukaryotic promoter. In some embodiments, the eukaryotic promoter comprises nucleic catabolite activator protein (CAP) binding site or a lactose (lac) promoter. In some embodiments, the eukaryotic selecting region comprises a lac operator. In some embodiments the plasmid comprises an M13 reverse primer region.

### AAV Virion

In some embodiments, a recombinant AAV virion is provided. In some embodiments, the AAV virion comprises an AAV capsid protein and the recombinant AAV vector that comprises the nucleotide sequence encoding hPax4 operably linked to regulatory elements. In some embodiments, the recombinant AAV vector is a recombinant scAAV vector. In some embodiments, the recombinant AAV is a single-stranded DNA vector. In some embodiments, the AAV capsid protein encapsulates the recombinant AAV vector.

In some embodiments, the AAV capsid protein includes VP1, VP2, and/or VP3 capsid proteins. The capsid preferably has tropism for appropriate cells and tissues, such as, for example, pancreatic islet tissues. In some embodiments, the capsid protein comprises an AAV1 capsid protein, an AAV2 capsid protein, an AAV3 capsid protein, an AAV4 capsid protein, an AAV5 capsid protein, an AAV6 capsid protein, an AAV7 capsid protein, an AAV8 capsid protein, or an AAV9 capsid protein. In some embodiments, the rAAV particle has an AAV6 capsid protein, for example, having an amino acid sequence of SEQ ID NO: 19. Alternatively, the capsid protein has an amino acid sequence that is 99%, 98%, 95%, 90% or 85% identical to the AAV6 capsid and has the tropism and transduction activity of the AAV6 capsid protein. The AAV6 capsid protein may be encoded by the nucleotide sequence of SEQ ID NO: 20 (or is at least 80%, 85%, 90%, 95%, or 99% identical to SEQ ID NO: 20 and encodes the AAV6 capsid protein). In some embodiments, the rAAV particle has an AAV8 capsid protein, for example, having an amino acid sequence of SEQ ID NO: 23. Alternatively, the capsid protein has an amino acid sequence that is 99%, 98%, 95%, 90% or 85% identical to the AAV8 capsid and has the tropism and transduction activity of the AAV8 capsid protein. The AAV8 capsid protein may be encoded by the nucleotide sequence of SEQ ID NO: 24 (or is at least 80%, 85%, 90%, 95%, or 99% identical to SEQ ID NO: 24 and encodes the AAV8 capsid protein). In some embodiments, the rAAV particle has an AAV9 capsid protein, for example, having an amino acid sequence of SEQ ID NO: 21. Alternatively, the capsid protein has an amino acid sequence that is 99%, 98%, 95%, 90% or 85% identical to the AAV9 capsid and has the tropism and transduction activity of the AAV9 capsid protein. The AAV9 capsid protein may be encoded by the nucleotide sequence of SEQ ID NO: 22 (or is at least 80%, 85%, 90%, 95%, or 99% identical to SEQ ID NO: 22 and encodes the AAV9 capsid protein).

In some aspects, the isolated nucleic acids and/or rAAVs described herein can be modified and/or selected to enhance the targeting of the isolated rAAVs to a target tissue (e.g., pancreatic islet cells). Non-limiting methods of modifications and/or selections include AAV capsid serotypes (e.g., AAV6, AAV8 or AAV9), tissue-specific promoters, and/or targeting peptides. In some aspects, the isolated nucleic acids and rAAVs disclosed herein can comprise AAV capsid serotypes with enhanced targeting to islet cell tissues. In some aspects, the isolated nucleic acids and rAAVs described herein can comprise tissue-specific promoters. In some aspects, the isolated nucleic acids and rAAVs described herein can comprise AAV capsid serotypes with enhanced targeting to islet cells and tissue-specific promoters.

Methods for obtaining recombinant AAVs having a desired capsid protein can be obtained from U.S. Patent Application Publication Number 2003/0138772, for example, which is incorporated herein by reference in its entirety. Typically, the methods involve culturing a host cell which contains a nucleic acid sequence encoding an AAV capsid protein or fragment thereof; a functional rep gene; sufficient helper functions to permit packaging of the recombinant AAV vector into the AAV capsid proteins; and a recombinant AAV vector plasmid comprising the AAV vector. Typically, capsid proteins are structural proteins encoded by the cap gene of an AAV. In some aspects, wherein the capsid protein comprises VP1, VP2, and VP3, said VP1, VP2, and VP3 are transcribed from a single cap gene via alternative splicing. In some aspects, the molecular weights of VP1, VP2 and VP3 are respectively about 87 kDa, about 72 kDa and about 62 kDa. In some aspects, upon translation, capsid proteins form a spherical 60-mer protein shell around the viral genome. In some aspects, capsid proteins protect a viral genome, deliver a genome and/or interact with a host cell. In some aspects, capsid proteins deliver the viral genome to a host in a tissue specific manner.

In some aspects, components to be cultured in the host cell to package a recombinant AAV vector in an AAV capsid can be provided to the host cell in trans. Alternatively, any one or more of the required components (e.g., recombinant AAV vector, rep sequences, cap sequences, and/or helper functions) can be provided by a stable host cell which has been engineered to contain one or more of the required components. In particular embodiments, provided are host cells comprising the recombinant AAV constructs or plasmids comprising the target gene sequence, a plasmid providing the AAV rep and cap gene sequences, and a construct providing adenoviral helper proteins as needed to produce the recombinant viral particle.

In some aspects, such a stable host cell can contain the required component(s) under the control of an inducible promoter. However, the required component(s) can be under the control of a constitutive promoter. Examples of suitable inducible and constitutive promoters are provided herein, in the discussion of regulatory elements suitable for use with the transgene. In some aspects, a selected stable host cell can contain selected component(s) under the control of a constitutive promoter and other selected component(s) under the control of one or more inducible promoters. For example, a stable host cell may be generated which is derived from 293 cells (which contain El helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions useful for producing the rAAV described herein can be delivered to the packaging host cell using any appropriate genetic element (vector, e.g. plasmid). The selected genetic element can be delivered by any suitable method, including those described herein. The methods used to construct any of compositions disclosed herein are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745.

In some aspects, recombinant AAVs can be produced using the triple transfection method (described in detail in U.S. Pat. No. 6,001,650). Typically, the recombinant AAVs can be produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation with the cap gene encoding the capsid proteins of desired serotype, for example, encoding the AAV9 capsid. In some aspects, the AAV helper function vector can support efficient AAV vector production without generating any detectable wild-type AAV virions (i.e., AAV virions containing functional rep and cap genes). Non-limiting examples of vectors suitable for use with the present disclosure include piLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

Cells. Disclosed herein are transfected host cells. The term “transfection” is used to refer to the uptake of foreign DNA by a cell, and a cell has been “transfected” when exogenous DNA has been introduced through the cell membrane. Examples of methods of transfection include Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

In one aspect, a cell is provided and may be a mammalian host cell, for example, HEK293 cells or other appropriate cells. In some embodiments, the cell comprises an AAV second plasmid (or repcap plasmid) and an AAV vector plasmid and, in some embodiments, a helper or accessory plasmid encoding adenoviral helper factors useful or necessary for production of AAV particles in the cell. In some embodiments, the AAV second plasmid comprises rep and cap. In some embodiments, the cap encodes a VP1, a VP2, and a VP3. In some embodiments, the rep encodes rep78, rep68, rep 52, and rep 40. In some embodiments, the AAV vector plasmid comprises the recombinant AAV vector or the recombinant scAAV vector. In some embodiments, the cap is AAV6 cap, AAV8 cap, or AAV9 cap, for example, having an AAV6 capsid protein with an amino acid sequence of SEQ ID NO: 19, or is encoded by the nucleotide sequence of SEQ ID NO: 20, or is AAV8 cap, for example having an AAV8 capsid protein with an amino acid sequence of SEQ ID NO: 23, or encoded by a nucleotide sequence of SEQ ID NO: 24, or is AAV9 cap, for example having an AAV9 capsid protein with an amino acid sequence of SEQ ID NO: 21, or encoded by a nucleotide sequence of SEQ ID NO: 22. In some embodiments, the AAV vector plasmid comprises the expression cassette of SEQ ID NO: 10, SEQ ID NO:11, or SEQ ID NO: 12 or may be pscAAV-CAG-hPax4-KanR (FIG. 1, SEQ ID NO: 16), pscAAV-MGP-hPax4, KanR (FIG. 2, SEQ ID NO: 17), or pscAAV-RIP-hPax4-KanR (FIG. 3, SEQ ID NO: 18).

In another aspect, a method of producing the AAV virion is provided. In some embodiments, the method comprises transfecting a cell with a second plasmid and at least one of a vector plasmid or the AAV vector construct. In some embodiments, the vector plasmid or AAV vector construct comprises the recombinant AAV vector or the recombinant scAAV vector. In some embodiments, the second plasmid comprises cap and rep. In some embodiments, the cap encodes the VP1, the VP2, and the VP3. In some embodiments, the rep encodes rep78, rep68, rep 52, and rep 40. In some embodiments, the vector plasmid or the AAV vector construct comprises the expression cassette having the nucleotide sequence of SEQ ID NO: 10, SEQ ID NO: 11, or SEQ ID NO: 12, (no flanking ITRs) or having the nucleotide sequence of SEQ ID NO: 13, SEQ ID NO: 14, or SEQ ID NO: 15 (with flanking ITRs). The plasmids used include pscAAV-CAG-hPax4-KanR (FIG. 1, SEQ ID NO: 16), pscAAV-MGP-hPax4, KanR (FIG. 2, SEQ ID NO: 17), and pscAAV-RIP-hPax4-KanR (FIG. 3, SEQ ID NO: 18).

### Methods of Use

Provided are methods of delivering Pax4 (including human Pax4) to a cell by introducing a Pax4 expressing vector disclosed herein into the cell such that the Pax4 nucleotide sequence is expressed in the cell. Embodiments include administration to a subject a recombinant AAV vector comprising an expression cassette comprising a nucleotide sequence encoding Pax4 operably linked to regulatory elements to promote expression of Pax4 in cells of the subject. In some embodiments, the cells are pancreatic cells, including islet cells, further including, α-cells and/or β-cells. In some embodiments, the regulatory elements promote Pax4 expression specifically in α-cells and/or β-cells.

Although not intending to be held to a particular mechanism of action, expression of Pax4 in pancreatic islet cells can promote β-cell function and increase β-cell numbers in the pancreas through promoting proliferation and/or survival of β-cells and/or transdifferentiation of α-cells into β-cells. The β-cells exhibit glucose-stimulated insulin secretion, resulting in improved blood glucose levels and glucose tolerance, reducing the risk of sequelae of diabetic disease in the subject or reducing the risk of a pre-diabetic subject developing diabetes.

Accordingly, provided herein are methods of increasing insulin production in cells, including pancreatic islet cells, including β-cells, in a subject in need thereof by administering an effective amount of AAV virions disclosed herein. In some embodiments, the methods disclosed result in an increase in fasting insulin levels of 20%, 50%, 100% or 2 fold, 5 fold or 10 fold relative to the insulin levels in the subject prior to administration under comparable conditions within 1 month, 2 months, 4 months, 6 months, 8 months or 1 year of administration of the rAAV. Also provided are methods of increasing the numbers of 3-cells in the pancreas and/or increasing the proportion of β-cells, including the ratio of β-cells to α-cells, within the islet cells of the pancreas of a subject in need thereof. The number of β-cells may be increased by 2-fold, 10-fold, 50-fold, 100-fold, 1000-fold or greater. In some embodiments, the methods promote transdifferentiation of α-cells to β-cells in the pancreas of a subject in need thereof.

In accordance therewith, provided are methods of treating, ameliorating the symptoms of, or slowing the progression of diabetes by administering effective amounts of the AAV virions comprising Pax4 transgenes disclosed herein in a subject in need thereof. The methods provide for, within 1 month, 2 months, 4 months, 6 months, 8 months or 1 year of administration, an improvement in blood glucose levels (either fasting levels or in response to a standard glucose challenge), glucose tolerance, and/or increasing insulin levels (fasting and/or in response to glucose challenge) in a subject in need thereof. Such methods include improvements in measurements of blood glucose and/or glucose tolerance, C-peptide levels, A1C levels, etc.

In some embodiments, the improvement in blood glucose levels, glucose tolerance testing and/or insulin secretion achieved by the gene therapy methods described herein are in comparison to the individual subject prior to the treatment with the AAV virions of the present disclosure. In some embodiments, treatment according to the present disclosure results in metrics at or around pre-diabetic levels within one month, 2 months, 3 months, 4 months, 5 months or 6 months after administration. In some embodiments, treatment according to the present disclosure results in fasting blood glucose levels of 100 to 125 mg/dL, or even below 100 mg/dL. In some embodiments, treatment according to the present disclosure results in glucose tolerance test (GTT) levels of 140 to 199 mg/dL, or even below 140 mg/dL. In some embodiments, the treatment according to the present disclosure results in a C-peptide level between about 0.5 ng/mL to about 2.0 ng/mL or about 0.17 nmol/L to about 0.83 nmol/L. In some embodiments, the improvement in A1C levels results in an A1C level between 5.7 and 6.5%.

The methods disclosed herein are useful in the treatment, amelioration, and/or reducing the symptoms of diabetes, including Type 1 diabetes (T1D) or Type 2 diabetes (T2D). In some embodiments, the methods include preventing progression of disease, including reducing or slowing the progression of pre-diabetes to diabetes or worsening of T1D or T2D. In other embodiments, provided are methods of preventing or slowing the progression of pre-diabetes to diabetes, including type 1 diabetes, in a subject having pre-diabetes indicators, such as predisposition to diabetes (including a close family member diagnosed with type 1 diabetes), or presence of autoantibodies against islet cells, including against GAD65, IA-2 and/or insulin. In other embodiments, provided are methods of preventing or slowing the progression of pre-diabetes to diabetes (including Type 2 diabetes), where the pre-diabetes is characterized by a fasting blood sugar level from 100 to 125 mg/dL, or a blood sugar level in a glucose tolerance test of 140 to 199 mg/dL or and A1C level between 5.7% to 6.4%.

Subjects may be human or non-human mammals, including rodents, dogs, cats, horses, livestock, etc. In the case of a non-human subject, the Pax4 may be of the species being treated, for example, a Pax4 from a mouse is used in administering the therapy to a mouse, for example.

In some embodiments, the administration of the rAAV comprising an expression cassette encoding Pax4 as described herein reduces the incidence of or severity of the complications or sequelae of type 1 or type 2 diabetes. In some embodiments, the method comprises administering to the subject in need of treatment a therapeutically effective amount of the AAV virion, including a pharmaceutical composition comprising the AAV virion, which ameliorates, reduces the severity of or progression of complications of diabetes such as, but not limited to, neuropathy, hypertension, kidney disease, cardiovascular disease, skin infections, hearing impairment, or retinopathy.

In some embodiments, the administering or treating comprises systemic administration, including, intravenous administration, intra-arterial, intramuscular administration, intraarticular administration, intraperitoneal administration, intradermal administration, subcutaneous administration, transdermal administration, or transmucosal administration. In some embodiments, the rAAV is delivered directly to the pancreas, for example, intraductal administration to the pancreas, (also called intra-bile ductal (IBD) administration) or endoscopic retrograde cholangio-pancreatography (ERCP).

In some embodiments, an effective amount of an rAAV virion can be an amount sufficient to cause a therapeutic effect, such as an increase in insulin production or improvement in a metric associated with T1D or T2D, such as blood glucose levels, C peptide levels or A1C levels. The effective amount will depend primarily on factors such as the species, age, weight, health of the subject, and the tissue to be targeted, and may thus vary among animal and tissue. For example, an effective amount of the rAAV virion can be in the range from about 1 ml to about 100 ml of solution containing from about 10⁶ to 10¹⁶ genome copies (e.g., from 1×10⁶ to 1×10¹⁶, inclusive). In methods disclosed herein, the therapeutically effective dose is between 1×10¹⁰ gc/kg to 1×10¹⁵ gc/kg, including 1×10¹¹ gc/kg, 1×10¹² gc/kg, 1×10¹³ gc/kg, 1×10¹⁴ gc/kg, 1×10¹⁵ gc/kg (or alternatively, genome copies per pancreatic cell or islet cell volume or other measurement appropriate for pancreatic delivery delivery) or an absolute number of rAAV virions 1×10⁶ to 1×10¹⁶, inclusive. In some aspects, a dosage of between about 10¹¹ to 10¹³ per kg or appropriate measurement rAAV genome copies can be appropriate. In some aspects, a dosage of between about 10¹¹ to 10¹⁴ per kg or appropriate measurement rAAV genome copies can be appropriate. In some aspects, a dosage of between about 10¹¹ to 10¹⁵ per kg or appropriate measurement rAAV genome copies can be appropriate. In some aspects, a dosage of about 1×10¹⁴ vector genome (vg) copies per kg or appropriate measurement can be appropriate.

In some embodiments, the administering or treating can comprise at least, or no more than, 1, 2, 3, 4, 5, 6, 7, 8, 9, or 10 administrations or treatments. In some embodiments, treatment consists of one administration of a therapeutically effective amount of the rAAV particle. In some embodiments, successful treatment and/or repair is determined when one or more of the following is detected: alleviation or amelioration of one or more of symptoms of the treated subject's disease, disorder, or condition, diminishment of extent of the subject's disease, disorder, or condition, stabilized (i.e., not worsening) state of a disease, disorder, or condition, delay or slowing of the progression of the disease, disorder, or condition, and amelioration or palliation of the disease, disorder, or condition. In some embodiments, success of treatment is determined by detecting the presence repaired target polynucleotide in one or more cells, tissues, or organs isolated from the subject. In some embodiments, success of treatment is determined by detecting the presence polypeptide encoded by the repaired target polynucleotide in one or more cells, tissues, or organs isolated from the subject. In some embodiments, success of treatment is determined by detecting a reduction in fasting blood sugar level compared to before treatment and, in some embodiments, to a level from 100 to 125 mg/dL. In some embodiments, success of treatment is determined by detecting a reduction in fasting blood sugar level compared to before treatment and, in some embodiments, to a level from 90 to 125 mg/dL. In some embodiments, success of treatment is determined by detecting a reduction in blood sugar level in a glucose tolerance test compared to before treatment and, in some embodiments, to a level of 140 to 199 mg/dL. In some embodiments, success of treatment is determined by detecting a reduction in A1C level compared to before treatment and, in some embodiments, to a level between 5.7% to 6.4%

In some embodiments, the recombinant AAV (rAAV) virion can be administered in a composition, which optionally comprises a suitable carrier. Suitable carriers can be selected for the indication for which the rAAV is directed. For example, one suitable carrier includes saline, which may be formulated with a variety of buffering solutions (e.g., phosphate buffered saline). Examples of other suitable carriers include but are not limited to sterile saline, lactose, sucrose, calcium phosphate, gelatin, dextran, agar, pectin, peanut oil, sesame oil, and water. Optionally, the compositions disclosed herein can also include, in addition to the rAAV virion and carrier(s), other pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

In particular embodiments, the rAAV virion is administered in a pharmaceutical composition comprising phosphate buffered saline (PBS), pH 7.3 and 0.001% of a pharmaceutically acceptable non-ionic surfactant, such as, for example, pluronic F-68 (PF68), or other appropriate pharmaceutically acceptable buffers or excipients. In some embodiments, the pharmaceutical composition can comprise the rAAV in an aqueous buffer comprising or consisting of 10 mM Tris, 150 mM NaCl, 0.02% poloxamer 188, 1 mM MgCl2, adjusted to a pH of 8.0.

In some aspects, the compositions disclosed herein can comprise a rAAV virion alone, or in combination with one or more other viruses (e.g., a second rAAV virion encoding having one or more different transgenes). In some aspects, a composition can comprise 1, 2, 3, 4, 5, 6, 7, 8, 9, 10, or more different rAAVs virion each having one or more different transgenes.

As used herein, “immunosuppressed” or “immunosuppression” refers to a decrease in the activation or efficacy of an immune response in a subject. Immunosuppression can be induced in a subject using one or more (e.g., multiple, such as 2, 3, 4, 5, or more) agents, including, but not limited to, rituximab, methylprednisolone, prednisolone, sirolimus, immunoglobulin injection, prednisone, methotrexate, an interleukin-6 inhibitor, an anti-interleukin-6 antibody, an interleukin-6 receptor inhibitor, an anti-interleukin-6 receptor antibody, and any combination thereof.

In some aspects, methods disclosed herein can further comprise the step of inducing immunosuppression (e.g., administering one or more immunosuppressive agents) in a subject prior to the subject being administered a rAAV virion (e.g., a rAAV virion or pharmaceutical composition as disclosed herein). In some aspects, a subject can be immunosuppressed (e.g., immunosuppression is induced in the subject) between about 30 days and about 0 days (e.g., any time between 30 days until administration of the rAAV virion, inclusive) prior to administration of the rAAV virion to the subject. In some aspects, the subject can be pre¬treated with immune suppression agent (e.g., rituximab, sirolimus, and/or prednisone) for at least 7 days.

In some aspects, immunosuppression of a subject is maintained during and/or after administration of a rAAV virion or pharmaceutical composition. In some aspects, a subject can be immunosuppressed (e.g., administered one or more immunosuppressants) for between 1 day and 1 year after administration of the rAAV virion or pharmaceutical composition.

For administration of an injectable aqueous solution, for example, the solution can be suitably buffered, if necessary, and the liquid diluent first rendered isotonic with sufficient saline or glucose. These particular aqueous solutions can be suitable for intraductal (pancreas), endoscopic retrograde cholangio-pancreatography (ERCP), intravenous, intramuscular, subcutaneous, and intraperitoneal administration. In this connection, a sterile aqueous medium can be employed. For example, one dosage can be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject. The person responsible for administration will, in any event, determine the appropriate dose for the individual subject.

Sterile injectable solutions can be prepared by incorporating the active rAAV virion in the required amount in the appropriate solvent with various of the other ingredients enumerated herein, as required, followed by filtered sterilization. Generally, dispersions can be prepared by incorporating the various sterilized active ingredients into a sterile vehicle which contains the basic dispersion medium and the required other ingredients from those enumerated above. In the case of sterile powders for the preparation of sterile injectable solutions, the methods of preparation can be vacuum-drying and freeze-drying techniques which yield a powder of the active ingredient plus any additional desired ingredient from a previously sterile-filtered solution thereof.

As used herein, the term “treating” refers to the application or administration of a composition (e.g., an rAAV as described herein) to a subject who has a disease or disorder associated with low levels of β-cells or insulin production, with the purpose to cure, heal, alleviate, relieve, alter, remedy, ameliorate, improve, or affect the disorder, a symptom of the disease, or a predisposition toward a disease.

Alleviating a disease associated with reduced β-cell numbers and/or insulin production includes delaying the development or progression of the disease, or reducing disease severity. Alleviating the disease does not necessarily require curative results. As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

In particular, administration of the rAAV virions described herein to a human subject suffering from β-cell deficiency and/or insulin-deficiency will within 5 weeks, 10 weeks, 15 weeks, 20 weeks, 25 weeks, 30 weeks, 40 weeks, 50 weeks or 1 year after the administration result in reduction in one or more biomarkers or hallmarks of the disease. In some embodiments, the human subject suffering from β-cell deficiency and/or insulin-deficiency will, within 5 weeks, 10 weeks, 15 weeks, 20 weeks, 25 weeks, 30 weeks, 40 weeks, 50 weeks or 1 year after the administration, have a reduction in fasting blood sugar level, including to a level from 100 to 125 mg/dL. In some embodiments, the human subject suffering from β-cell deficiency and/or insulin-deficiency will, within 5 weeks, 10 weeks, 15 weeks, 20 weeks, 25 weeks, 30 weeks, 40 weeks, 50 weeks or 1 year after the administration, have a reduction in blood sugar level in a glucose tolerance test, including to a level of 140 to 199 mg/dL. In some embodiments, the human subject suffering from β-cell deficiency and/or insulin-deficiency will, within 5 weeks, 10 weeks, 15 weeks, 20 weeks, 25 weeks, 30 weeks, 40 weeks, 50 weeks or 1 year after the administration, have a reduction in A1C level, including to between 5.7% to 6.4%

“Development” or “progression” of a disease means initial manifestations and/or ensuing progression of the disease. Development of the disease can be detectable and assessed using standard clinical techniques as well known in the art. However, development also refers to progression that can be undetectable. As used herein the terms development or progression refer to the biological course of the symptoms. “Development” includes occurrence, recurrence, and onset.

In some aspects, the rAAV virions disclosed herein can be administered in sufficient amounts to transduce the cells of a desired tissue and to provide sufficient levels of gene transfer and expression without undue adverse effects. Pharmaceutically acceptable routes of administration include, but are not limited to, direct delivery to the selected organ (e.g., to the pancreas), by intraductal (including intra-bile ductal) administration or endoscopic retrograde cholangio-pancreatography (ERCP) administration. Other routes of administration include intravenous, intramuscular, subcutaneous, intradermal, and other parental routes of administration. Routes of administration can be combined, if desired.

### Kits

Disclosed herein are kits comprising any of the agents described herein. In some aspects, any of the agents disclosed herein can be assembled into pharmaceutical or diagnostic or research kits to facilitate their use in therapeutic, diagnostic or research applications. A kit can include one or more containers housing the components of the disclosure and instructions for use. Specifically, such kits may include one or more agents described herein, along with instructions describing the intended application and the proper use of these agents. In some aspects, the agents in a kit can be in a pharmaceutical formulation and dosage suitable for a particular application and for a method of administration of the agents. Kits for research purposes can contain the components in appropriate concentrations or quantities for running various experiments.

Also disclosed herein are kits for producing a rAAV virions. In some aspects, the kit can comprise a container housing an isolated nucleic acid encoding a Pax4 protein or a portion thereof. In some aspects, the kits can further comprise instructions for producing the rAAV virion. In some aspects, the kit further comprises at least one container housing a recombinant AAV vector, wherein the recombinant AAV vector comprises a transgene (i.e, Pax4).

In some aspects, the kits can comprise a container housing a recombinant AAV virion as described supra. In some aspects, the kits can further comprise a container housing a pharmaceutically acceptable carrier. For example, a kit can comprise one container housing a rAAV virion and a second container housing a buffer suitable for injection of the rAAV virion into a subject. In some aspects, the container can be a syringe.

In some aspects, the kits can be designed to facilitate use of the methods described herein by researchers and can take many forms. Each of the compositions of the kit, where applicable, may be provided in liquid form (e.g., in solution), or in solid form, (e.g., a dry powder). In some aspects, some of the compositions can be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions can be associated with the kit, for example, audiovisual (e.g., videotape, DVD, etc.), internet, and/or web-based communications, etc. The written instructions can be in a form prescribed by a governmental agency regulating the manufacture, use or sale of pharmaceuticals or biological products, which instructions can also reflect approval by the agency of manufacture, use or sale for animal administration.

The kits disclosed herein can also contain any one or more of the components described herein in one or more containers. In some aspects, the kits can include instructions for mixing one or more components of the kit and/or isolating and mixing a sample and applying to a subject. The kits can include a container housing agents described herein. The agents can be in the form of a liquid, gel or solid (powder). The agents can be prepared sterilely, packaged in syringe and shipped refrigerated. Alternatively, it can be housed in a vial or other container for storage. A second container can have other agents prepared sterilely. Alternatively, the kits can include the active agents premixed and shipped in a syringe, vial, tube, or other container. The kits can have one or more or all of the components required to administer the agents to an animal, such as a syringe, topical application devices, or iv needle tubing and bag, particularly in the case of the kits for producing specific somatic animal models.

The kits disclosed can have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box or a bag. The kits can be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits can also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The instructions included within the kit can involve methods for detecting a latent AAV in a cell. In addition, kits of the disclosure can include, instructions, a negative and/or positive control, containers, diluents and buffers for the sample, sample preparation tubes and a printed or electronic table of reference AAV sequence for sequence comparisons.

TABLE 1
Sequences of Construct Elements and Constructs

| Element | SEQ | |
|---|---|---|
| or | ID | |
| Construct | NO: | Sequence |
| Human | 1 | MNQLGGLFVNGRPLPLDTRQQIVRLAVSGMRPCDISRILKVSNGCVSKILGR |
| Pax4 | | YYRTGVLEPKGIGGSKPRLATPPVVARIAQLKGECPALFAWEIQRQLCAEGL |
| | | CTQDKTPSVSSINRVLRALQEDQGLPCTRLRSPAVLAPAVLTPHSGSETPRG |
| | | THPGTGHRNRTIFSPSQAEALEKEFQRGQYPDSVARGKLATATSLPEDTVRV |
| | | WFSNRRAKWRRQEKLKWEMQLPGASQGLTVPRVAPGIISAQQSPGSVPTAAL |
| | | PALEPLGPSCYQLCWATAPERCLSDTPPKACLKPCWGHLPPQPNSLDSGLLC |
| | | LPCPSSHCPLASLSGSQALLWPGCPLLYGLE* |
| Human | 2 | atgaaccagcttggggggctctttgtgaatggccggcccctgcctctggata |
| Pax4 | | cccggcagcagattgtgcggctagcagtcagtggaatgcggccctgtgacat |
| coding | | ctcacggatccttaaggtatctaatggctgtgtgagcaagatcctagggcgt |
| sequence | | tactaccgcacaggtgtcttggagccaaagggcattgggggaagcaagccac |
| | | ggctggctacaccccctgtggtggctcgaattgcccagctgaagggtgagtg |
| | | tccagccctctttgcctgggaaatccaacgccagctttgtgctgaagggctt |
| | | tgcacccaggacaagactcccagtgtctcctccatcaaccgagtcctgcggg |
| | | cattacaggaggaccagggactaccgtgcacacggctcaggtcaccagctgt |
| | | tttggctccagctgtcctcactccccatagtggctctgagactccccggggt |
| | | acccacccagggaccggccaccggaatcggactatcttctccccaagccagg |
| | | cagaggcactggagaaagagttccagcgtgggcagtatcctgattcagtggc |
| | | ccgtggaaagctggctactgccacctctctgcctgaggacacggtgagggtc |
| | | tggttttccaacagaagagccaaatggcgtcggcaagagaagctcaagtggg |
| | | aaatgcagctgccaggtgcttcccaggggctgactgtaccaagggttgcccc |
| | | aggaatcatctctgcacagcagtcccctggcagtgtgcccacagcagccctg |
| | | cctgccctggaaccactgggtccctcctgctatcagctgtgctgggcaacag |
| | | caccagaaaggtgtctgagtgacaccccacctaaagcctgtctcaagccctg |
| | | ctggggccacttgcccccacagccgaattccctggactcaggactgctttgc |
| | | cttccttgcccttcctcccactgtcccctggccagtcttagtggctctcagg |
| | | ccctgctctggcctggctgcccactactgtatggcttggaatga |
| Kozak | 3 | gccacc |
| Sequence | | |
| CAG | 4 | ccattgacgtcaataatgacgtatgttcccatagtaacgccaatagggactt |
| Promoter | | tccattgacgtcaatgggtggagtatttacggtaaactgcccacttggcagt |
| | | acatcaagtgtatcatatgccaagtacgccccctattgacgtcaatgacggt |
| | | aaatggcccgcctggcattatgcccagtacatgaccttatgggactttccta |
| | | cttggcagtacatctacgtattagtcatcgctattaccatggtcgaggtgag |
| | | ccccacgttctgcttcactctccccatctcccccccctccccacccccaatt |
| | | ttgtatttatttattttttaattattttgtgcagcgatgggggcgggggggg |
| | | ggggggggcgcgcgccaggcggggcggggcggggcgaggggcggggcggggc |
| | | gaggcggagaggtgcggcggcagccaatcagagcggcgcgctccgaaagttt |
| | | ccttttatggcgaggcggcggcggcggcggccctataaaaagcgaagcgcgc |
| | | ggcgggcgggagtcgctgcgcgctgccttcgccccgtgccccgctccgccgc |
| | | cgcctcgcgccgcccgccccggctctgactgaccgcgttactcccacaggtg |
| | | agcgggcgggacggcccttctcctccgggctgtaattagcgcttgg |
| Mouse | 5 | gagatatagccaaataccaaatcaagggataagaccctcaaatgagactagg |
| Glucagon | | ctcatttgacgtcaaaattcacttgagagaactttagcagtttttcgtgcct |
| Promoter | | gactgagaccgaagggtggatctccaaactgccctttccattcccaaacaga |
| | | aaggcacaagagtaaataaaatgtttccgggcctctgcggtctcaacccggt |
| | | atcagcgtaaaaagcagatgagcaaagtgagtgggcgagtgaaatcatttga |
| | | acaaaaccccattatttacagatgagaaatttatattgtcagcgtaatatct |
| | | gcaaggctaaacagcctggagagcatataaaagcacagcaccctggtgcaga |
| | | agggcagagcttgggcccaggacacactcaaagttcccaaggggactccctc |
| | | tgtctagat |
| Rat | 6 | gctgagctaagaatccagctatcaatagaaactatgaaacagttccagggac |
| Insulin | | aaagataccaggtccccaacaactgcaactttctgggaaatgaggtggaaaa |
| Promoter | | tgctcagccaaggaaaaagagggccttaccctctctgggacaatgattgtgc |
| | | tgtgaactgcttcatcaggccatctggccccttgttaataatctaattaccc |
| | | taggtctaagtagagttgttgacgtccaatgagcgctttctgcagacttagc |
| | | actaggcaagtgtttggaaattacagcttcagcccctctcgccatctgccta |
| | | cctacccctcctagagcccttaatgggccaaacggcaaagtccagggggcag |
| | | agaggaggtgctttggactataaagctagtggagacccagtaactccca |
| SV40 | 7 | aacttgtttattgcagcttataatggttacaaataaagcaatagcatcacaa |
| PolyA | | atttcacaaataaagcatttttttcactgcattctagttgtggtttgtccaa |
| signal | | actcatcaatgtatctta |
| Sequence | | |
| 5′ ITR | 8 | ttggccactccctctctgcgcgctcgctcgctcactgaggccgcccgggcaa |
| | | agcccgggcgtcgggcgacctttggtcgcccggcctcagtgagcgagcgagc |
| | | gcgcagagagggagtggccaactccatcactaggggttcct |
| scITR (3′) | 9 | ccactccctctctgcgcgctcgctcgctcactgaggccgggcgaccaaaggt |
| | | cgcccgacgcccgggctttgcccgggcggcctcagtgagcgagcgagcgcgc |
| | | ag |
| 3′ ITR | 25 | aggaacccctagtgatggagttggccactccctctctgcgcgctcgctcgct |
| | | cactgaggccgggcgaccaaaggtcgcccgacgcccgggctttgcccgggcg |
| | | gcctcagtgagcgagcgagcgcgcagagagggagtggccaa |
| CAG- | 10 | ccattgacgtcaataatgacgtatgttcccatagtaacgccaatagggactt |
| hPax4 | | tccattgacgtcaatgggtggagtatttacggtaaactgcccacttggcagt |
| expression | | acatcaagtgtatcatatgccaagtacgccccctattgacgtcaatgacggt |
| cassette | | aaatggcccgcctggcattatgcccagtacatgaccttatgggactttccta |
| (promoter | | cttggcagtacatctacgtattagtcatcgctattaccatggtcgaggtgag |
| to polyA | | ccccacgttctgcttcactctccccatctcccccccctccccacccccaatt |
| signal) | | ttgtatttatttattttttaattattttgtgcagcgatgggggcgggggggg |
| | | ggggggggcgcgcgccaggcggggcggggcggggcgaggggcggggcggggc |
| | | gaggcggagaggtgcggcggcagccaatcagagcggcgcgctccgaaagttt |
| | | ccttttatggcgaggcggcggcggcggcggccctataaaaagcgaagcgcgc |
| | | ggcgggcgggagtcgctgcgcgctgccttcgccccgtgccccgctccgccgc |
| | | cgcctcgcgccgcccgccccggctctgactgaccgcgttactcccacaggtg |
| | | agcgggcgggacggcccttctcctccgggctgtaattagcgcttggagatct |
| | | ggtaccgtcgacgccaccatgaaccagcttggggggctctttgtgaatggcc |
| | | ggcccctgcctctggatacccggcagcagattgtgcggctagcagtcagtgg |
| | | aatgcggccctgtgacatctcacggatccttaaggtatctaatggctgtgtg |
| | | agcaagatcctagggcgttactaccgcacaggtgtcttggagccaaagggca |
| | | ttgggggaagcaagccacggctggctacaccccctgtggtggctcgaattgc |
| | | ccagctgaagggtgagtgtccagccctctttgcctgggaaatccaacgccag |
| | | ctttgtgctgaagggctttgcacccaggacaagactcccagtgtctcctcca |
| | | tcaaccgagtcctgcgggcattacaggaggaccagggactaccgtgcacacg |
| | | gctcaggtcaccagctgttttggctccagctgtcctcactccccatagtggc |
| | | tctgagactccccggggtacccacccagggaccggccaccggaatcggacta |
| | | tcttctccccaagccaggcagaggcactggagaaagagttccagcgtgggca |
| | | gtatcctgattcagtggcccgtggaaagctggctactgccacctctctgcct |
| | | gaggacacggtgagggtctggttttccaacagaagagccaaatggcgtcggc |
| | | aagagaagctcaagtgggaaatgcagctgccaggtgcttcccaggggctgac |
| | | tgtaccaagggttgccccaggaatcatctctgcacagcagtcccctggcagt |
| | | gtgcccacagcagccctgcctgccctggaaccactgggtccctcctgctatc |
| | | agctgtgctgggcaacagcaccagaaaggtgtctgagtgacaccccacctaa |
| | | agcctgtctcaagccctgctggggccacttgcccccacagccgaattccctg |
| | | gactcaggactgctttgccttccttgcccttcctcccactgtcccctggcca |
| | | gtcttagtggctctcaggccctgctctggcctggctgcccactactgtatgg |
| | | cttggaatgaagcgctgctcgagagatctacaacttgtttattgcagcttat |
| | | aatggttacaaataaagcaatagcatcacaaatttcacaaataaagcatttt |
| | | tttcactgcattctagttgtggtttgtccaaactcatcaatgtatctta |
| MGP- | 11 | gagatatagccaaataccaaatcaagggataagaccctcaaatgagactagg |
| hPax4 | | ctcatttgacgtcaaaattcacttgagagaactttagcagtttttcgtgcct |
| expression | | gactgagaccgaagggtggatctccaaactgccctttccattcccaaacaga |
| cassette | | aaggcacaagagtaaataaaatgtttccgggcctctgcggtctcaacccggt |
| (promoter | | atcagcgtaaaaagcagatgagcaaagtgagtgggcgagtgaaatcatttga |
| to polyA | | acaaaaccccattatttacagatgagaaatttatattgtcagcgtaatatct |
| signal) | | gcaaggctaaacagcctggagagcatataaaagcacagcaccctggtgcaga |
| | | agggcagagcttgggcccaggacacactcaaagttcccaaggggactccctc |
| | | tgtctagatatctggtaccgtcgacgccaccatgaaccagcttggggggctc |
| | | tttgtgaatggccggcccctgcctctggatacccggcagcagattgtgcggc |
| | | tagcagtcagtggaatgcggccctgtgacatctcacggatccttaaggtatc |
| | | taatggctgtgtgagcaagatcctagggcgttactaccgcacaggtgtcttg |
| | | gagccaaagggcattgggggaagcaagccacggctggctacaccccctgtgg |
| | | tggctcgaattgcccagctgaagggtgagtgtccagccctctttgcctggga |
| | | aatccaacgccagctttgtgctgaagggctttgcacccaggacaagactccc |
| | | agtgtctcctccatcaaccgagtcctgcgggcattacaggaggaccagggac |
| | | taccgtgcacacggctcaggtcaccagctgttttggctccagctgtcctcac |
| | | tccccatagtggctctgagactccccggggtacccacccagggaccggccac |
| | | cggaatcggactatcttctccccaagccaggcagaggcactggagaaagagt |
| | | tccagcgtgggcagtatcctgattcagtggcccgtggaaagctggctactgc |
| | | cacctctctgcctgaggacacggtgagggtctggttttccaacagaagagcc |
| | | aaatggcgtcggcaagagaagctcaagtgggaaatgcagctgccaggtgctt |
| | | cccaggggctgactgtaccaagggttgccccaggaatcatctctgcacagca |
| | | gtcccctggcagtgtgcccacagcagccctgcctgccctggaaccactgggt |
| | | ccctcctgctatcagctgtgctgggcaacagcaccagaaaggtgtctgagtg |
| | | acaccccacctaaagcctgtctcaagccctgctggggccacttgcccccaca |
| | | gccgaattccctggactcaggactgctttgccttccttgcccttcctcccac |
| | | tgtcccctggccagtcttagtggctctcaggccctgctctggcctggctgcc |
| | | cactactgtatggcttggaatgaagcgctgctcgagagatctacaacttgtt |
| | | tattgcagcttataatggttacaaataaagcaatagcatcacaaatttcaca |
| | | aataaagcatttttttcactgcattctagttgtggtttgtccaaactcatca |
| | | atgtatctta |
| RIP- | 12 | gctgagctaagaatccagctatcaatagaaactatgaaacagttccagggac |
| hPax4 | | aaagataccaggtccccaacaactgcaactttctgggaaatgaggtggaaaa |
| expression | | tgctcagccaaggaaaaagagggccttaccctctctgggacaatgattgtgc |
| cassette | | tgtgaactgcttcatcaggccatctggccccttgttaataatctaattaccc |
| (promoter | | taggtctaagtagagttgttgacgtccaatgagcgctttctgcagacttagc |
| to polyA | | actaggcaagtgtttggaaattacagcttcagcccctctcgccatctgccta |
| signal) | | cctacccctcctagagcccttaatgggccaaacggcaaagtccagggggcag |
| | | agaggaggtgctttggactataaagctagtggagacccagtaactcccaaga |
| | | tctggtaccgtcgacgccaccatgaaccagcttggggggctctttgtgaatg |
| | | gccggcccctgcctctggatacccggcagcagattgtgcggctagcagtcag |
| | | tggaatgcggccctgtgacatctcacggatccttaaggtatctaatggctgt |
| | | gtgagcaagatcctagggcgttactaccgcacaggtgtcttggagccaaagg |
| | | gcattgggggaagcaagccacggctggctacaccccctgtggtggctcgaat |
| | | tgcccagctgaagggtgagtgtccagccctctttgcctgggaaatccaacgc |
| | | cagctttgtgctgaagggctttgcacccaggacaagactcccagtgtctcct |
| | | ccatcaaccgagtcctgcgggcattacaggaggaccagggactaccgtgcac |
| | | acggctcaggtcaccagctgttttggctccagctgtcctcactccccatagt |
| | | ggctctgagactccccggggtacccacccagggaccggccaccggaatcgga |
| | | ctatcttctccccaagccaggcagaggcactggagaaagagttccagcgtgg |
| | | gcagtatcctgattcagtggcccgtggaaagctggctactgccacctctctg |
| | | cctgaggacacggtgagggtctggttttccaacagaagagccaaatggcgtc |
| | | ggcaagagaagctcaagtgggaaatgcagctgccaggtgcttcccaggggct |
| | | gactgtaccaagggttgccccaggaatcatctctgcacagcagtcccctggc |
| | | agtgtgcccacagcagccctgcctgccctggaaccactgggtccctcctgct |
| | | atcagctgtgctgggcaacagcaccagaaaggtgtctgagtgacaccccacc |
| | | taaagcctgtctcaagccctgctggggccacttgcccccacagccgaattcc |
| | | ctggactcaggactgctttgccttccttgcccttcctcccactgtcccctgg |
| | | ccagtcttagtggctctcaggccctgctctggcctggctgcccactactgta |
| | | tggcttggaatgaagcgctgctcgagagatctacaacttgtttattgcagct |
| | | tataatggttacaaataaagcaatagcatcacaaatttcacaaataaagcat |
| | | ttttttcactgcattctagttgtggtttgtccaaactcatcaatgtatctta |
| scAAV- | 13 | ttggccactccctctctgcgcgctcgctcgctcactgaggccgcccgggcaa |
| CAG- | | agcccgggcgtcgggcgacctttggtcgcccggcctcagtgagcgagcgagc |
| hPax4 | | gcgcagagagggagtggccaactccatcactaggggttcctgatgtaatacg |
| genome | | actcactagttcctggaggggtggagtcgtgacctaggccattgacgtcaat |
| (ITR to | | aatgacgtatgttcccatagtaacgccaatagggactttccattgacgtcaa |
| ITR) | | tgggtggagtatttacggtaaactgcccacttggcagtacatcaagtgtatc |
| | | atatgccaagtacgccccctattgacgtcaatgacggtaaatggcccgcctg |
| | | gcattatgcccagtacatgaccttatgggactttcctacttggcagtacatc |
| | | tacgtattagtcatcgctattaccatggtcgaggtgagccccacgttctgct |
| | | tcactctccccatctcccccccctccccacccccaattttgtatttatttat |
| | | tttttaattattttgtgcagcgatgggggcggggggggggggggggcgcgcg |
| | | ccaggcggggcggggcggggcgaggggcggggcggggcgaggcggagaggtg |
| | | cggcggcagccaatcagagcggcgcgctccgaaagtttccttttatggcgag |
| | | gcggcggcggcggcggccctataaaaagcgaagcgcgcggcgggcgggagtc |
| | | gctgcgcgctgccttcgccccgtgccccgctccgccgccgcctcgcgccgcc |
| | | cgccccggctctgactgaccgcgttactcccacaggtgagcgggcgggacgg |
| | | cccttctcctccgggctgtaattagcgcttggagatctggtaccgtcgacgc |
| | | caccatgaaccagcttggggggctctttgtgaatggccggcccctgcctctg |
| | | gatacccggcagcagattgtgcggctagcagtcagtggaatgcggccctgtg |
| | | acatctcacggatccttaaggtatctaatggctgtgtgagcaagatcctagg |
| | | gcgttactaccgcacaggtgtcttggagccaaagggcattgggggaagcaag |
| | | ccacggctggctacaccccctgtggtggctcgaattgcccagctgaagggtg |
| | | agtgtccagccctctttgcctgggaaatccaacgccagctttgtgctgaagg |
| | | gctttgcacccaggacaagactcccagtgtctcctccatcaaccgagtcctg |
| | | cgggcattacaggaggaccagggactaccgtgcacacggctcaggtcaccag |
| | | ctgttttggctccagctgtcctcactccccatagtggctctgagactccccg |
| | | gggtacccacccagggaccggccaccggaatcggactatcttctccccaagc |
| | | caggcagaggcactggagaaagagttccagcgtgggcagtatcctgattcag |
| | | tggcccgtggaaagctggctactgccacctctctgcctgaggacacggtgag |
| | | ggtctggttttccaacagaagagccaaatggcgtcggcaagagaagctcaag |
| | | tgggaaatgcagctgccaggtgcttcccaggggctgactgtaccaagggttg |
| | | ccccaggaatcatctctgcacagcagtcccctggcagtgtgcccacagcagc |
| | | cctgcctgccctggaaccactgggtccctcctgctatcagctgtgctgggca |
| | | acagcaccagaaaggtgtctgagtgacaccccacctaaagcctgtctcaagc |
| | | cctgctggggccacttgcccccacagccgaattccctggactcaggactgct |
| | | ttgccttccttgcccttcctcccactgtcccctggccagtcttagtggctct |
| | | caggccctgctctggcctggctgcccactactgtatggcttggaatgaagcg |
| | | ctgctcgagagatctacaacttgtttattgcagcttataatggttacaaata |
| | | aagcaatagcatcacaaatttcacaaataaagcatttttttcactgcattct |
| | | agttgtggtttgtccaaactcatcaatgtatcttaaggcctaggtgagctct |
| | | ggtaccctctccactccctctctgcgcgctcgctcgctcactgaggccgggc |
| | | gaccaaaggtcgcccgacgcccgggctttgcccgggcggcctcagtgagcga |
| | | gcgagcgcgcag |
| SCAAV- | 14 | ttggccactccctctctgcgcgctcgctcgctcactgaggccgcccgggcaa |
| MGP- | | agcccgggcgtcgggcgacctttggtcgcccggcctcagtgagcgagcgagc |
| Pax4 | | gcgcagagagggagtggccaactccatcactaggggttcctgatgtaatacg |
| genome | | actcactagttcctggaggggtggagtcgtgacctagggagatatagccaaa |
| (ITR to | | taccaaatcaagggataagaccctcaaatgagactaggctcatttgacgtca |
| ITR | | aaattcacttgagagaactttagcagtttttcgtgcctgactgagaccgaag |
| | | ggtggatctccaaactgccctttccattcccaaacagaaaggcacaagagta |
| | | aataaaatgtttccgggcctctgcggtctcaacccggtatcagcgtaaaaag |
| | | cagatgagcaaagtgagtgggcgagtgaaatcatttgaacaaaaccccatta |
| | | tttacagatgagaaatttatattgtcagcgtaatatctgcaaggctaaacag |
| | | cctggagagcatataaaagcacagcaccctggtgcagaagggcagagcttgg |
| | | gcccaggacacactcaaagttcccaaggggactccctctgtctagatatctg |
| | | gtaccgtcgacgccaccatgaaccagcttggggggctctttgtgaatggccg |
| | | gcccctgcctctggatacccggcagcagattgtgcggctagcagtcagtgga |
| | | atgcggccctgtgacatctcacggatccttaaggtatctaatggctgtgtga |
| | | gcaagatcctagggcgttactaccgcacaggtgtcttggagccaaagggcat |
| | | tgggggaagcaagccacggctggctacaccccctgtggtggctcgaattgcc |
| | | cagctgaagggtgagtgtccagccctctttgcctgggaaatccaacgccagc |
| | | tttgtgctgaagggctttgcacccaggacaagactcccagtgtctcctccat |
| | | caaccgagtcctgcgggcattacaggaggaccagggactaccgtgcacacgg |
| | | ctcaggtcaccagctgttttggctccagctgtcctcactccccatagtggct |
| | | ctgagactccccggggtacccacccagggaccggccaccggaatcggactat |
| | | cttctccccaagccaggcagaggcactggagaaagagttccagcgtgggcag |
| | | tatcctgattcagtggcccgtggaaagctggctactgccacctctctgcctg |
| | | aggacacggtgagggtctggttttccaacagaagagccaaatggcgtcggca |
| | | agagaagctcaagtgggaaatgcagctgccaggtgcttcccaggggctgact |
| | | gtaccaagggttgccccaggaatcatctctgcacagcagtcccctggcagtg |
| | | tgcccacagcagccctgcctgccctggaaccactgggtccctcctgctatca |
| | | gctgtgctgggcaacagcaccagaaaggtgtctgagtgacaccccacctaaa |
| | | gcctgtctcaagccctgctggggccacttgcccccacagccgaattccctgg |
| | | actcaggactgctttgccttccttgcccttcctcccactgtcccctggccag |
| | | tcttagtggctctcaggccctgctctggcctggctgcccactactgtatggc |
| | | ttggaatgaagcgctgctcgagagatctacaacttgtttattgcagcttata |
| | | atggttacaaataaagcaatagcatcacaaatttcacaaataaagcattttt |
| | | ttcactgcattctagttgtggtttgtccaaactcatcaatgtatcttaaggc |
| | | ctaggtgagctctggtaccctctccactccctctctgcgcgctcgctcgctc |
| | | actgaggccgggcgaccaaaggtcgcccgacgcccgggctttgcccgggcgg |
| | | cctcagtgagcgagcgagcgcgcag |
| scAAV- | 15 | ttggccactccctctctgcgcgctcgctcgctcactgaggccgcccgggcaa |
| RIP- | | agcccgggcgtcgggcgacctttggtcgcccggcctcagtgagcgagcgagc |
| hPax4 | | gcgcagagagggagtggccaactccatcactaggggttcctgatgtaatacg |
| (ITR to | | actcactagttcctggaggggtggagtcgtgacctagggctgagctaagaat |
| ITR) | | ccagctatcaatagaaactatgaaacagttccagggacaaagataccaggtc |
| | | cccaacaactgcaactttctgggaaatgaggtggaaaatgctcagccaagga |
| | | aaaagagggccttaccctctctgggacaatgattgtgctgtgaactgcttca |
| | | tcaggccatctggccccttgttaataatctaattaccctaggtctaagtaga |
| | | gttgttgacgtccaatgagcgctttctgcagacttagcactaggcaagtgtt |
| | | tggaaattacagcttcagcccctctcgccatctgcctacctacccctcctag |
| | | agcccttaatgggccaaacggcaaagtccagggggcagagaggaggtgcttt |
| | | ggactataaagctagtggagacccagtaactcccaagatctggtaccgtcga |
| | | cgccaccatgaaccagcttggggggctctttgtgaatggccggcccctgcct |
| | | ctggatacccggcagcagattgtgcggctagcagtcagtggaatgcggccct |
| | | gtgacatctcacggatccttaaggtatctaatggctgtgtgagcaagatcct |
| | | agggcgttactaccgcacaggtgtcttggagccaaagggcattgggggaagc |
| | | aagccacggctggctacaccccctgtggtggctcgaattgcccagctgaagg |
| | | gtgagtgtccagccctctttgcctgggaaatccaacgccagctttgtgctga |
| | | agggctttgcacccaggacaagactcccagtgtctcctccatcaaccgagtc |
| | | ctgcgggcattacaggaggaccagggactaccgtgcacacggctcaggtcac |
| | | cagctgttttggctccagctgtcctcactccccatagtggctctgagactcc |
| | | ccggggtacccacccagggaccggccaccggaatcggactatcttctcccca |
| | | agccaggcagaggcactggagaaagagttccagcgtgggcagtatcctgatt |
| | | cagtggcccgtggaaagctggctactgccacctctctgcctgaggacacggt |
| | | gagggtctggttttccaacagaagagccaaatggcgtcggcaagagaagctc |
| | | aagtgggaaatgcagctgccaggtgcttcccaggggctgactgtaccaaggg |
| | | ttgccccaggaatcatctctgcacagcagtcccctggcagtgtgcccacagc |
| | | agccctgcctgccctggaaccactgggtccctcctgctatcagctgtgctgg |
| | | gcaacagcaccagaaaggtgtctgagtgacaccccacctaaagcctgtctca |
| | | agccctgctggggccacttgcccccacagccgaattccctggactcaggact |
| | | gctttgccttccttgcccttcctcccactgtcccctggccagtcttagtggc |
| | | tctcaggccctgctctggcctggctgcccactactgtatggcttggaatgaa |
| | | gcgctgctcgagagatctacaacttgtttattgcagcttataatggttacaa |
| | | ataaagcaatagcatcacaaatttcacaaataaagcatttttttcactgcat |
| | | tctagttgtggtttgtccaaactcatcaatgtatcttaaggcctaggtgagc |
| | | tctggtaccctctccactccctctctgcgcgctcgctcgctcactgaggccg |
| | | ggcgaccaaaggtcgcccgacgcccgggctttgcccgggcggcctcagtgag |
| | | cgagcgagcgcgcag |
| pscAAV- | 16 | ggcgcgcccaaaggctatcaggtcattgcctgagagtctggagcaaacaaga |
| CAG- | | gaatcgttggccactccctctctgcgcgctcgctcgctcactgaggccgccc |
| hPax4- | | gggcaaagcccgggcgtcgggcgacctttggtcgcccggcctcagtgagcga |
| KanR | | gcgagcgcgcagagagggagtggccaactccatcactaggggttcctgatgt |
| | | aatacgactcactagttcctggaggggtggagtcgtgacctaggccattgac |
| | | gtcaataatgacgtatgttcccatagtaacgccaatagggactttccattga |
| | | cgtcaatgggtggagtatttacggtaaactgcccacttggcagtacatcaag |
| | | tgtatcatatgccaagtacgccccctattgacgtcaatgacggtaaatggcc |
| | | cgcctggcattatgcccagtacatgaccttatgggactttcctacttggcag |
| | | tacatctacgtattagtcatcgctattaccatggtcgaggtgagccccacgt |
| | | tctgcttcactctccccatctcccccccctccccacccccaattttgtattt |
| | | atttattttttaattattttgtgcagcgatgggggcgggggggggggggggg |
| | | cgcgcgccaggcggggcggggcggggcgaggggcggggcggggcgaggcgga |
| | | gaggtgcggcggcagccaatcagagcggcgcgctccgaaagtttccttttat |
| | | ggcgaggcggcggcggcggcggccctataaaaagcgaagcgcgcggcgggcg |
| | | ggagtcgctgcgcgctgccttcgccccgtgccccgctccgccgccgcctcgc |
| | | gccgcccgccccggctctgactgaccgcgttactcccacaggtgagcgggcg |
| | | ggacggcccttctcctccgggctgtaattagcgcttggagatctggtaccgt |
| | | cgacgccaccatgaaccagcttggggggctctttgtgaatggccggcccctg |
| | | cctctggatacccggcagcagattgtgcggctagcagtcagtggaatgcggc |
| | | cctgtgacatctcacggatccttaaggtatctaatggctgtgtgagcaagat |
| | | cctagggcgttactaccgcacaggtgtcttggagccaaagggcattggggga |
| | | agcaagccacggctggctacaccccctgtggtggctcgaattgcccagctga |
| | | agggtgagtgtccagccctctttgcctgggaaatccaacgccagctttgtgc |
| | | tgaagggctttgcacccaggacaagactcccagtgtctcctccatcaaccga |
| | | gtcctgcgggcattacaggaggaccagggactaccgtgcacacggctcaggt |
| | | caccagctgttttggctccagctgtcctcactccccatagtggctctgagac |
| | | tccccggggtacccacccagggaccggccaccggaatcggactatcttctcc |
| | | ccaagccaggcagaggcactggagaaagagttccagcgtgggcagtatcctg |
| | | attcagtggcccgtggaaagctggctactgccacctctctgcctgaggacac |
| | | ggtgagggtctggttttccaacagaagagccaaatggcgtcggcaagagaag |
| | | ctcaagtgggaaatgcagctgccaggtgcttcccaggggctgactgtaccaa |
| | | gggttgccccaggaatcatctctgcacagcagtcccctggcagtgtgcccac |
| | | agcagccctgcctgccctggaaccactgggtccctcctgctatcagctgtgc |
| | | tgggcaacagcaccagaaaggtgtctgagtgacaccccacctaaagcctgtc |
| | | tcaagccctgctggggccacttgcccccacagccgaattccctggactcagg |
| | | actgctttgccttccttgcccttcctcccactgtcccctggccagtcttagt |
| | | ggctctcaggccctgctctggcctggctgcccactactgtatggcttggaat |
| | | gaagcgctgctcgagagatctacaacttgtttattgcagcttataatggtta |
| | | caaataaagcaatagcatcacaaatttcacaaataaagcatttttttcactg |
| | | cattctagttgtggtttgtccaaactcatcaatgtatcttaaggcctaggtg |
| | | agctctggtaccctctccactccctctctgcgcgctcgctcgctcactgagg |
| | | ccgggcgaccaaaggtcgcccgacgcccgggctttgcccgggcggcctcagt |
| | | gagcgagcgagcgcgcagggcgatgaacggtaatcgtaaaactagcatgtca |
| | | atcatatgtaccccggttgataatcagaaaagccccaaaaacaggaagattg |
| | | tataagcaaatatttaaattgtaagcgttaatattttgttaaaattcgcgtt |
| | | aaatttttgttaaatcagctcattttttaaccaataggccgaaatcggcaaa |
| | | atcccttataaatcaaaagaatagaccgagatagggttgagtgttgttccag |
| | | tttggaacaagagtccactattaaagaacgtggactccaacgtcaaagggcg |
| | | aaaaaccgtctatcagggcgatggcccactacgtgaaccatcaccctaatca |
| | | agttttttggggtcgaggtgccgtaaagcactaaatcggaaccctaaaggga |
| | | gcccccgatttagagcttgacggggaaagccggcgaacgtggcgagaaagga |
| | | agggaagaaagcgaaaggagcgggcgctagggcgctggcaagtgtagcggtc |
| | | acgctgcgcgtaaccaccacacccgccgcgcttaatgcgccgctacagggcg |
| | | cgtactatggttgctttgacgagcacgtataacgtgctttcctcgttagaat |
| | | cagagcgggagctaaacaggaggccgattaaagggattttagacaggaacgg |
| | | tacgccagaatcctgagaagtgtttttataatcagtgaggccaccgagtaaa |
| | | agagtctgtccatcacgcaaattaaccgttgtcgcaatacttctttgattag |
| | | taataacatcacttgcctgagtagaagaactcaaactatcggccttgctggt |
| | | aatatccagaacaatattaccgccagccattgcaacggaatcgccattcgcc |
| | | attcaggctgcgcaactgttgggaagggcgatcggtgcgggcctcttcgcta |
| | | ttacttaattaacatggtcatagctgtttcctgtgtgaaattgttatccgct |
| | | cacaattccacacaacatacgagccggaagcataaagtgtaaagcctggggt |
| | | gcctaatgagtgagctaactcacattaattgcgttgcgctcactgcccgctt |
| | | tccagtcgggaaacctgtcgtgccagctgcattaatgaatcggccaacgcgc |
| | | ggggagaggcggtttgcgtattgggcgctcttccgcttcctcgctcactgac |
| | | tcgctgcgctcggtcgttcggctgcggcgagcggtatcagctcactcaaagg |
| | | cggtaatacggttatccacagaatcaggggataacgcaggaaagaacatgtg |
| | | agcaaaaggccagcaaaaggccaggaaccgtaaaaaggccgcgttgctggcg |
| | | tttttccataggctccgcccccctgacgagcatcacaaaaatcgacgctcaa |
| | | gtcagaggtggcgaaacccgacaggactataaagataccaggcgtttccccc |
| | | tggaagctccctcgtgcgctctcctgttccgaccctgccgcttaccggatac |
| | | ctgtccgcctttctcccttcgggaagcgtggcgctttctcatagctcacgct |
| | | gtaggtatctcagttcggtgtaggtcgttcgctccaagctgggctgtgtgca |
| | | cgaaccccccgttcagcccgaccgctgcgccttatccggtaactatcgtctt |
| | | gagtccaacccggtaagacacgacttatcgccactggcagcagccactggta |
| | | acaggattagcagagcgaggtatgtaggcggtgctacagagttcttgaagtg |
| | | gtggcctaactacggctacactagaagaacagtatttggtatctgcgctctg |
| | | ctgaagccagttaccttcggaaaaagagttggtagctcttgatccggcaaac |
| | | aaaccaccgctggtagcggtggtttttttgtttgcaagcagcagattacgcg |
| | | cagaaaaaaaggatctcaagaagatcctttgatcttttctacggggtctgac |
| | | gctcagtggaacgaaaactcacgttaagggattttggtcatgagattatcaa |
| | | aaaggatcttcacctagatccttttaaattaaaaatgaagttttaaatcaat |
| | | ctaaagtatatatgagtaaacttggtctgacagttagaaaaactcatcgagc |
| | | atcaaatgaaactgcaatttattcatatcaggattatcaataccatattttt |
| | | gaaaaagccgtttctgtaatgaaggagaaaactcaccgaggcagttccatag |
| | | gatggcaagatcctggtatcggtctgcgattccgactcgtccaacatcaata |
| | | caacctattaatttcccctcgtcaaaaataaggttatcaagtgagaaatcac |
| | | catgagtgacgactgaatccggtgagaatggcaaaagtttatgcatttcttt |
| | | ccagacttgttcaacaggccagccattacgctcgtcatcaaaatcactcgca |
| | | tcaaccaaaccgttattcattcgtgattgcgcctgagcgagacgaaatacgc |
| | | gatcgctgttaaaaggacaattacaaacaggaatcgaatgcaaccggcgcag |
| | | gaacactgccagcgcatcaacaatattttcacctgaatcaggatattcttct |
| | | aatacctggaatgctgtttttccggggatcgcagtggtgagtaaccatgcat |
| | | catcaggagtacggataaaatgcttgatggtcggaagaggcataaattccgt |
| | | cagccagtttagtctgaccatctcatctgtaacatcattggcaacgctacct |
| | | ttgccatgtttcagaaacaactctggcgcatcgggcttcccatacaagcgat |
| | | agattgtcgcacctgattgcccgacattatcgcgagcccatttatacccata |
| | | taaatcagcatccatgttggaatttaatcgcggcctcgacgtttcccgttga |
| | | atatggctcatactcttcctttttcaatattattgaagcatttatcagggtt |
| | | attgtctcatgagcggatacatatttgaatgtatttagaaaaataaacaaat |
| | | aggggttccgcgcacatttccccgaaaagtgccacctgacgtctaagaaacc |
| | | attattatcatgacattaacctataaaaataggcgtatcacgaggccctttc |
| | | gtctcgcgcgtttcggtgatgacggtgaaaacctctgacacatgcagctccc |
| | | ggagacggtcacagcttgtctgtaagcggatgccgggagcagacaagcccgt |
| | | cagggcgcgtcagcgggtgttggcgggtgtcggggctggcttaactatgcgg |
| | | catcagagcagattgtactgagagtgcaccatatgcggtgtgaaataccgca |
| | | cagatgcgtaaggagaaaataccgcatcaggcgccattcgccattcaggctg |
| | | cgcaactgttgggaagggcgatcggtgcgggcctcttcgctattacgccagc |
| | | tggcgaaagggggatgtgctgcaaggcgattaagttgggtaacgccagggtt |
| | | ttcccagtcacgacgttgtaaaacgacggccagta |
| pscAAV- | 17 | ggcgcgcccaaaggctatcaggtcattgcctgagagtctggagcaaacaaga |
| MGP- | | gaatcgttggccactccctctctgcgcgctcgctcgctcactgaggccgccc |
| hPax4- | | gggcaaagcccgggcgtcgggcgacctttggtcgcccggcctcagtgagcga |
| KanR | | gcgagcgcgcagagagggagtggccaactccatcactaggggttcctgatgt |
| | | aatacgactcactagttcctggaggggtggagtcgtgacctagggagatata |
| | | gccaaataccaaatcaagggataagaccctcaaatgagactaggctcatttg |
| | | acgtcaaaattcacttgagagaactttagcagtttttcgtgcctgactgaga |
| | | ccgaagggtggatctccaaactgccctttccattcccaaacagaaaggcaca |
| | | agagtaaataaaatgtttccgggcctctgcggtctcaacccggtatcagcgt |
| | | aaaaagcagatgagcaaagtgagtgggcgagtgaaatcatttgaacaaaacc |
| | | ccattatttacagatgagaaatttatattgtcagcgtaatatctgcaaggct |
| | | aaacagcctggagagcatataaaagcacagcaccctggtgcagaagggcaga |
| | | gcttgggcccaggacacactcaaagttcccaaggggactccctctgtctaga |
| | | tatctggtaccgtcgacgccaccatgaaccagcttggggggctctttgtgaa |
| | | tggccggcccctgcctctggatacccggcagcagattgtgcggctagcagtc |
| | | agtggaatgcggccctgtgacatctcacggatccttaaggtatctaatggct |
| | | gtgtgagcaagatcctagggcgttactaccgcacaggtgtcttggagccaaa |
| | | gggcattgggggaagcaagccacggctggctacaccccctgtggtggctcga |
| | | attgcccagctgaagggtgagtgtccagccctctttgcctgggaaatccaac |
| | | gccagctttgtgctgaagggctttgcacccaggacaagactcccagtgtctc |
| | | ctccatcaaccgagtcctgcgggcattacaggaggaccagggactaccgtgc |
| | | acacggctcaggtcaccagctgttttggctccagctgtcctcactccccata |
| | | gtggctctgagactccccggggtacccacccagggaccggccaccggaatcg |
| | | gactatcttctccccaagccaggcagaggcactggagaaagagttccagcgt |
| | | gggcagtatcctgattcagtggcccgtggaaagctggctactgccacctctc |
| | | tgcctgaggacacggtgagggtctggttttccaacagaagagccaaatggcg |
| | | tcggcaagagaagctcaagtgggaaatgcagctgccaggtgcttcccagggg |
| | | ctgactgtaccaagggttgccccaggaatcatctctgcacagcagtcccctg |
| | | gcagtgtgcccacagcagccctgcctgccctggaaccactgggtccctcctg |
| | | ctatcagctgtgctgggcaacagcaccagaaaggtgtctgagtgacacccca |
| | | cctaaagcctgtctcaagccctgctggggccacttgcccccacagccgaatt |
| | | ccctggactcaggactgctttgccttccttgcccttcctcccactgtcccct |
| | | ggccagtcttagtggctctcaggccctgctctggcctggctgcccactactg |
| | | tatggcttggaatgaagcgctgctcgagagatctacaacttgtttattgcag |
| | | cttataatggttacaaataaagcaatagcatcacaaatttcacaaataaagc |
| | | atttttttcactgcattctagttgtggtttgtccaaactcatcaatgtatct |
| | | taaggcctaggtgagctctggtaccctctccactccctctctgcgcgctcgc |
| | | tcgctcactgaggccgggcgaccaaaggtcgcccgacgcccgggctttgccc |
| | | gggcggcctcagtgagcgagcgagcgcgcagggcgatgaacggtaatcgtaa |
| | | aactagcatgtcaatcatatgtaccccggttgataatcagaaaagccccaaa |
| | | aacaggaagattgtataagcaaatatttaaattgtaagcgttaatattttgt |
| | | taaaattcgcgttaaatttttgttaaatcagctcattttttaaccaataggc |
| | | cgaaatcggcaaaatcccttataaatcaaaagaatagaccgagatagggttg |
| | | agtgttgttccagtttggaacaagagtccactattaaagaacgtggactcca |
| | | acgtcaaagggcgaaaaaccgtctatcagggcgatggcccactacgtgaacc |
| | | atcaccctaatcaagttttttggggtcgaggtgccgtaaagcactaaatcgg |
| | | aaccctaaagggagcccccgatttagagcttgacggggaaagccggcgaacg |
| | | tggcgagaaaggaagggaagaaagcgaaaggagcgggcgctagggcgctggc |
| | | aagtgtagcggtcacgctgcgcgtaaccaccacacccgccgcgcttaatgcg |
| | | ccgctacagggcgcgtactatggttgctttgacgagcacgtataacgtgctt |
| | | tcctcgttagaatcagagcgggagctaaacaggaggccgattaaagggattt |
| | | tagacaggaacggtacgccagaatcctgagaagtgtttttataatcagtgag |
| | | gccaccgagtaaaagagtctgtccatcacgcaaattaaccgttgtcgcaata |
| | | cttctttgattagtaataacatcacttgcctgagtagaagaactcaaactat |
| | | cggccttgctggtaatatccagaacaatattaccgccagccattgcaacgga |
| | | atcgccattcgccattcaggctgcgcaactgttgggaagggcgatcggtgcg |
| | | ggcctcttcgctattacttaattaacatggtcatagctgtttcctgtgtgaa |
| | | attgttatccgctcacaattccacacaacatacgagccggaagcataaagtg |
| | | taaagcctggggtgcctaatgagtgagctaactcacattaattgcgttgcgc |
| | | tcactgcccgctttccagtcgggaaacctgtcgtgccagctgcattaatgaa |
| | | tcggccaacgcgcggggagaggcggtttgcgtattgggcgctcttccgcttc |
| | | ctcgctcactgactcgctgcgctcggtcgttcggctgcggcgagcggtatca |
| | | gctcactcaaaggcggtaatacggttatccacagaatcaggggataacgcag |
| | | gaaagaacatgtgagcaaaaggccagcaaaaggccaggaaccgtaaaaaggc |
| | | cgcgttgctggcgtttttccataggctccgcccccctgacgagcatcacaaa |
| | | aatcgacgctcaagtcagaggtggcgaaacccgacaggactataaagatacc |
| | | aggcgtttccccctggaagctccctcgtgcgctctcctgttccgaccctgcc |
| | | gcttaccggatacctgtccgcctttctcccttcgggaagcgtggcgctttct |
| | | catagctcacgctgtaggtatctcagttcggtgtaggtcgttcgctccaagc |
| | | tgggctgtgtgcacgaaccccccgttcagcccgaccgctgcgccttatccgg |
| | | taactatcgtcttgagtccaacccggtaagacacgacttatcgccactggca |
| | | gcagccactggtaacaggattagcagagcgaggtatgtaggcggtgctacag |
| | | agttcttgaagtggtggcctaactacggctacactagaagaacagtatttgg |
| | | tatctgcgctctgctgaagccagttaccttcggaaaaagagttggtagctct |
| | | tgatccggcaaacaaaccaccgctggtagcggtggtttttttgtttgcaagc |
| | | agcagattacgcgcagaaaaaaaggatctcaagaagatcctttgatcttttc |
| | | tacggggtctgacgctcagtggaacgaaaactcacgttaagggattttggtc |
| | | atgagattatcaaaaaggatcttcacctagatccttttaaattaaaaatgaa |
| | | gttttaaatcaatctaaagtatatatgagtaaacttggtctgacagttagaa |
| | | aaactcatcgagcatcaaatgaaactgcaatttattcatatcaggattatca |
| | | ataccatatttttgaaaaagccgtttctgtaatgaaggagaaaactcaccga |
| | | ggcagttccataggatggcaagatcctggtatcggtctgcgattccgactcg |
| | | tccaacatcaatacaacctattaatttcccctcgtcaaaaataaggttatca |
| | | agtgagaaatcaccatgagtgacgactgaatccggtgagaatggcaaaagtt |
| | | tatgcatttctttccagacttgttcaacaggccagccattacgctcgtcatc |
| | | aaaatcactcgcatcaaccaaaccgttattcattcgtgattgcgcctgagcg |
| | | agacgaaatacgcgatcgctgttaaaaggacaattacaaacaggaatcgaat |
| | | gcaaccggcgcaggaacactgccagcgcatcaacaatattttcacctgaatc |
| | | aggatattcttctaatacctggaatgctgtttttccggggatcgcagtggtg |
| | | agtaaccatgcatcatcaggagtacggataaaatgcttgatggtcggaagag |
| | | gcataaattccgtcagccagtttagtctgaccatctcatctgtaacatcatt |
| | | ggcaacgctacctttgccatgtttcagaaacaactctggcgcatcgggcttc |
| | | ccatacaagcgatagattgtcgcacctgattgcccgacattatcgcgagccc |
| | | atttatacccatataaatcagcatccatgttggaatttaatcgcggcctcga |
| | | cgtttcccgttgaatatggctcatactcttcctttttcaatattattgaagc |
| | | atttatcagggttattgtctcatgagcggatacatatttgaatgtatttaga |
| | | aaaataaacaaataggggttccgcgcacatttccccgaaaagtgccacctga |
| | | cgtctaagaaaccattattatcatgacattaacctataaaaataggcgtatc |
| | | acgaggccctttcgtctcgcgcgtttcggtgatgacggtgaaaacctctgac |
| | | acatgcagctcccggagacggtcacagcttgtctgtaagcggatgccgggag |
| | | cagacaagcccgtcagggcgcgtcagcgggtgttggcgggtgtcggggctgg |
| | | cttaactatgcggcatcagagcagattgtactgagagtgcaccatatgcggt |
| | | gtgaaataccgcacagatgcgtaaggagaaaataccgcatcaggcgccattc |
| | | gccattcaggctgcgcaactgttgggaagggcgatcggtgcgggcctcttcg |
| | | ctattacgccagctggcgaaagggggatgtgctgcaaggcgattaagttggg |
| | | taacgccagggttttcccagtcacgacgttgtaaaacgacggccagta |
| pscAAV- | 18 | ggcgcgcccaaaggctatcaggtcattgcctgagagtctggagcaaacaaga |
| RIP- | | gaatcgttggccactccctctctgcgcgctcgctcgctcactgaggccgccc |
| hPax4- | | gggcaaagcccgggcgtcgggcgacctttggtcgcccggcctcagtgagcga |
| KanR | | gcgagcgcgcagagagggagtggccaactccatcactaggggttcctgatgt |
| | | aatacgactcactagttcctggaggggtggagtcgtgacctagggctgagct |
| | | aagaatccagctatcaatagaaactatgaaacagttccagggacaaagatac |
| | | caggtccccaacaactgcaactttctgggaaatgaggtggaaaatgctcagc |
| | | caaggaaaaagagggccttaccctctctgggacaatgattgtgctgtgaact |
| | | gcttcatcaggccatctggccccttgttaataatctaattaccctaggtcta |
| | | agtagagttgttgacgtccaatgagcgctttctgcagacttagcactaggca |
| | | agtgtttggaaattacagcttcagcccctctcgccatctgcctacctacccc |
| | | tcctagagcccttaatgggccaaacggcaaagtccagggggcagagaggagg |
| | | tgctttggactataaagctagtggagacccagtaactcccaagatctggtac |
| | | cgtcgacgccaccatgaaccagcttggggggctctttgtgaatggccggccc |
| | | ctgcctctggatacccggcagcagattgtgcggctagcagtcagtggaatgc |
| | | ggccctgtgacatctcacggatccttaaggtatctaatggctgtgtgagcaa |
| | | gatcctagggcgttactaccgcacaggtgtcttggagccaaagggcattggg |
| | | ggaagcaagccacggctggctacaccccctgtggtggctcgaattgcccagc |
| | | tgaagggtgagtgtccagccctctttgcctgggaaatccaacgccagctttg |
| | | tgctgaagggctttgcacccaggacaagactcccagtgtctcctccatcaac |
| | | cgagtcctgcgggcattacaggaggaccagggactaccgtgcacacggctca |
| | | ggtcaccagctgttttggctccagctgtcctcactccccatagtggctctga |
| | | gactccccggggtacccacccagggaccggccaccggaatcggactatcttc |
| | | tccccaagccaggcagaggcactggagaaagagttccagcgtgggcagtatc |
| | | ctgattcagtggcccgtggaaagctggctactgccacctctctgcctgagga |
| | | cacggtgagggtctggttttccaacagaagagccaaatggcgtcggcaagag |
| | | aagctcaagtgggaaatgcagctgccaggtgcttcccaggggctgactgtac |
| | | caagggttgccccaggaatcatctctgcacagcagtcccctggcagtgtgcc |
| | | cacagcagccctgcctgccctggaaccactgggtccctcctgctatcagctg |
| | | tgctgggcaacagcaccagaaaggtgtctgagtgacaccccacctaaagcct |
| | | gtctcaagccctgctggggccacttgcccccacagccgaattccctggactc |
| | | aggactgctttgccttccttgcccttcctcccactgtcccctggccagtctt |
| | | agtggctctcaggccctgctctggcctggctgcccactactgtatggcttgg |
| | | aatgaagcgctgctcgagagatctacaacttgtttattgcagcttataatgg |
| | | ttacaaataaagcaatagcatcacaaatttcacaaataaagcatttttttca |
| | | ctgcattctagttgtggtttgtccaaactcatcaatgtatcttaaggcctag |
| | | gtgagctctggtaccctctccactccctctctgcgcgctcgctcgctcactg |
| | | aggccgggcgaccaaaggtcgcccgacgcccgggctttgcccgggcggcctc |
| | | agtgagcgagcgagcgcgcagggcgatgaacggtaatcgtaaaactagcatg |
| | | tcaatcatatgtaccccggttgataatcagaaaagccccaaaaacaggaaga |
| | | ttgtataagcaaatatttaaattgtaagcgttaatattttgttaaaattcgc |
| | | gttaaatttttgttaaatcagctcattttttaaccaataggccgaaatcggc |
| | | aaaatcccttataaatcaaaagaatagaccgagatagggttgagtgttgttc |
| | | cagtttggaacaagagtccactattaaagaacgtggactccaacgtcaaagg |
| | | gcgaaaaaccgtctatcagggcgatggcccactacgtgaaccatcaccctaa |
| | | tcaagttttttggggtcgaggtgccgtaaagcactaaatcggaaccctaaag |
| | | ggagcccccgatttagagcttgacggggaaagccggcgaacgtggcgagaaa |
| | | ggaagggaagaaagcgaaaggagcgggcgctagggcgctggcaagtgtagcg |
| | | gtcacgctgcgcgtaaccaccacacccgccgcgcttaatgcgccgctacagg |
| | | gcgcgtactatggttgctttgacgagcacgtataacgtgctttcctcgttag |
| | | aatcagagcgggagctaaacaggaggccgattaaagggattttagacaggaa |
| | | cggtacgccagaatcctgagaagtgtttttataatcagtgaggccaccgagt |
| | | aaaagagtctgtccatcacgcaaattaaccgttgtcgcaatacttctttgat |
| | | tagtaataacatcacttgcctgagtagaagaactcaaactatcggccttgct |
| | | ggtaatatccagaacaatattaccgccagccattgcaacggaatcgccattc |
| | | gccattcaggctgcgcaactgttgggaagggcgatcggtgcgggcctcttcg |
| | | ctattacttaattaacatggtcatagctgtttcctgtgtgaaattgttatcc |
| | | gctcacaattccacacaacatacgagccggaagcataaagtgtaaagcctgg |
| | | ggtgcctaatgagtgagctaactcacattaattgcgttgcgctcactgcccg |
| | | ctttccagtcgggaaacctgtcgtgccagctgcattaatgaatcggccaacg |
| | | cgcggggagaggcggtttgcgtattgggcgctcttccgcttcctcgctcact |
| | | gactcgctgcgctcggtcgttcggctgcggcgagcggtatcagctcactcaa |
| | | aggcggtaatacggttatccacagaatcaggggataacgcaggaaagaacat |
| | | gtgagcaaaaggccagcaaaaggccaggaaccgtaaaaaggccgcgttgctg |
| | | gcgtttttccataggctccgcccccctgacgagcatcacaaaaatcgacgct |
| | | caagtcagaggtggcgaaacccgacaggactataaagataccaggcgtttcc |
| | | ccctggaagctccctcgtgcgctctcctgttccgaccctgccgcttaccgga |
| | | tacctgtccgcctttctcccttcgggaagcgtggcgctttctcatagctcac |
| | | gctgtaggtatctcagttcggtgtaggtcgttcgctccaagctgggctgtgt |
| | | gcacgaaccccccgttcagcccgaccgctgcgccttatccggtaactatcgt |
| | | cttgagtccaacccggtaagacacgacttatcgccactggcagcagccactg |
| | | gtaacaggattagcagagcgaggtatgtaggcggtgctacagagttcttgaa |
| | | gtggtggcctaactacggctacactagaagaacagtatttggtatctgcgct |
| | | ctgctgaagccagttaccttcggaaaaagagttggtagctcttgatccggca |
| | | aacaaaccaccgctggtagcggtggtttttttgtttgcaagcagcagattac |
| | | gcgcagaaaaaaaggatctcaagaagatcctttgatcttttctacggggtct |
| | | gacgctcagtggaacgaaaactcacgttaagggattttggtcatgagattat |
| | | caaaaaggatcttcacctagatccttttaaattaaaaatgaagttttaaatc |
| | | aatctaaagtatatatgagtaaacttggtctgacagttagaaaaactcatcg |
| | | agcatcaaatgaaactgcaatttattcatatcaggattatcaataccatatt |
| | | tttgaaaaagccgtttctgtaatgaaggagaaaactcaccgaggcagttcca |
| | | taggatggcaagatcctggtatcggtctgcgattccgactcgtccaacatca |
| | | atacaacctattaatttcccctcgtcaaaaataaggttatcaagtgagaaat |
| | | caccatgagtgacgactgaatccggtgagaatggcaaaagtttatgcatttc |
| | | tttccagacttgttcaacaggccagccattacgctcgtcatcaaaatcactc |
| | | gcatcaaccaaaccgttattcattcgtgattgcgcctgagcgagacgaaata |
| | | cgcgatcgctgttaaaaggacaattacaaacaggaatcgaatgcaaccggcg |
| | | caggaacactgccagcgcatcaacaatattttcacctgaatcaggatattct |
| | | tctaatacctggaatgctgtttttccggggatcgcagtggtgagtaaccatg |
| | | catcatcaggagtacggataaaatgcttgatggtcggaagaggcataaattc |
| | | cgtcagccagtttagtctgaccatctcatctgtaacatcattggcaacgcta |
| | | cctttgccatgtttcagaaacaactctggcgcatcgggcttcccatacaagc |
| | | gatagattgtcgcacctgattgcccgacattatcgcgagcccatttataccc |
| | | atataaatcagcatccatgttggaatttaatcgcggcctcgacgtttcccgt |
| | | tgaatatggctcatactcttcctttttcaatattattgaagcatttatcagg |
| | | gttattgtctcatgagcggatacatatttgaatgtatttagaaaaataaaca |
| | | aataggggttccgcgcacatttccccgaaaagtgccacctgacgtctaagaa |
| | | accattattatcatgacattaacctataaaaataggcgtatcacgaggccct |
| | | ttcgtctcgcgcgtttcggtgatgacggtgaaaacctctgacacatgcagct |
| | | cccggagacggtcacagcttgtctgtaagcggatgccgggagcagacaagcc |
| | | cgtcagggcgcgtcagcgggtgttggcgggtgtcggggctggcttaactatg |
| | | cggcatcagagcagattgtactgagagtgcaccatatgcggtgtgaaatacc |
| | | gcacagatgcgtaaggagaaaataccgcatcaggcgccattcgccattcagg |
| | | ctgcgcaactgttgggaagggcgatcggtgcgggcctcttcgctattacgcc |
| | | agctggcgaaagggggatgtgctgcaaggcgattaagttgggtaacgccagg |
| | | gttttcccagtcacgacgttgtaaaacgacggccagta |
| Capsids | | |
| AAV6 | 19 | MAADGYLPDW LEDNLSEGIR EWWDLKPGAP KPKANQQKQD |
| VP1 | | DGRGLVLPGY KYLGPENGLD KGEPVNAADA AALEHDKAYD |
| | | QQLKAGDNPY LRYNHADAEF QERLQEDTSF GGNLGRAVFQ |
| | | AKKRVLEPFG LVEEGAKTAP GKKRPVEQSP QEPDSSSGIG |
| | | KTGQQPAKKR LNFGQTGDSE SVPDPQPLGE PPATPAAVGP |
| | | TTMASGGGAP MADNNEGADG VGNASGNWHC DSTWLGDRVI |
| | | TTSTRTWALP TYNNHLYKQI SSASTGASND NHYFGYSTPW |
| | | GYFDFNRFHC HFSPRDWQRL INNNWGERPK RLNFKLFNIQ |
| | | VKEVTTNDGV TTIANNLTST VQVFSDSEYQ LPYVLGSAHQ |
| | | GCLPPFPADV FMIPQYGYLT LNNGSQAVGR SSFYCLEYFP |
| | | SQMLRTGNNF TFSYTFEDVP FHSSYAHSQS LDRLMNPLID |
| | | QYLYYLNRTQ NQSGSAQNKD LLFSRGSPAG MSVQPKNWLP |
| | | GPCYRQQRVS KTKTDNNNSN FTWTGASKYN LNGRESIINP |
| | | GTAMASHKDD KDKFFPMSGV MIFGKESAGA SNTALDNVMI |
| | | TDEEEIKATN PVATERFGTV AVNLQSSSTD PATGDVHVMG |
| | | ALPGMVWQDR DVYLQGPIWA KIPHTDGHFH PSPLMGGFGL |
| | | KHPPPQILIK NTPVPANPPA EFSATKFASF ITQYSTGQVS |
| | | VEIEWELQKE NSKRWNPEVQ YTSNYAKSAN VDFTVDNNGL |
| | | YTEPRPIGTR YLTRPL* |
| AAV6 | 20 | atggctgccgatggttatcttccagattggctcgaggacaacctctctgagg |
| VP1 | | gcattcgcgagtggtgggacttgaaacctggagccccgaaacccaaagccaa |
| coding | | ccagcaaaagcaggacgacggccggggtctggtgcttcctggctacaagtac |
| sequence | | ctcggacccttcaacggactcgacaagggggagcccgtcaacgcggcggatg |
| | | cagcggccctcgagcacgacaaggcctacgaccagcagctcaaagcgggtga |
| | | caatccgtacctgcggtataaccacgccgacgccgagtttcaggagcgtctg |
| | | caagaagatacgtcttttgggggcaacctcgggcgagcagtcttccaggcca |
| | | agaagagggttctcgaaccttttggtctggttgaggaaggtgctaagacggc |
| | | tcctggaaagaaacgtccggtagagcagtcgccacaagagccagactcctcc |
| | | tcgggcattggcaagacaggccagcagcccgctaaaaagagactcaattttg |
| | | gtcagactggcgactcagagtcagtccccgacccacaacctctcggagaacc |
| | | tccagcaacccccgctgctgtgggacctactacaatggcttcaggcggtggc |
| | | gcaccaatggcagacaataacgaaggcgccgacggagtgggtaatgcctcag |
| | | gaaattggcattgcgattccacatggctgggcgacagagtcatcaccaccag |
| | | cacccgaacatgggccttgcccacctataacaaccacctctacaagcaaatc |
| | | tccagtgcttcaacgggggccagcaacgacaaccactacttcggctacagca |
| | | ccccctgggggtattttgatttcaacagattccactgccatttctcaccacg |
| | | tgactggcagcgactcatcaacaacaattggggattccggcccaagagactc |
| | | aacttcaagctcttcaacatccaagtcaaggaggtcacgacgaatgatggcg |
| | | tcacgaccatcgctaataaccttaccagcacggttcaagtcttctcggactc |
| | | ggagtaccagttgccgtacgtcctcggctctgcgcaccagggctgcctccct |
| | | ccgttcccggcggacgtgttcatgattccgcagtacggctacctaacgctca |
| | | acaatggcagccaggcagtgggacggtcatccttttactgcctggaatattt |
| | | cccatcgcagatgctgagaacgggcaataactttaccttcagctacaccttc |
| | | gaggacgtgcctttccacagcagctacgcgcacagccagagcctggaccggc |
| | | tgatgaatcctctcatcgaccagtacctgtattacctgaacagaactcagaa |
| | | tcagtccggaagtgcccaaaacaaggacttgctgtttagccgggggtctcca |
| | | gctggcatgtctgttcagcccaaaaactggctacctggaccctgttaccggc |
| | | agcagcgcgtttctaaaacaaaaacagacaacaacaacagcaactttacctg |
| | | gactggtgcttcaaaatataaccttaatgggcgtgaatctataatcaaccct |
| | | ggcactgctatggcctcacacaaagacgacaaagacaagttctttcccatga |
| | | gcggtgtcatgatttttggaaaggagagcgccggagcttcaaacactgcatt |
| | | ggacaatgtcatgatcacagacgaagaggaaatcaaagccactaaccccgtg |
| | | gccaccgaaagatttgggactgtggcagtcaatctccagagcagcagcacag |
| | | accctgcgaccggagatgtgcatgttatgggagccttacctggaatggtgtg |
| | | gcaagacagagacgtatacctgcagggtcctatttgggccaaaattcctcac |
| | | acggatggacactttcacccgtctcctctcatgggcggctttggacttaagc |
| | | acccgcctcctcagatcctcatcaaaaacacgcctgttcctgcgaatcctcc |
| | | ggcagagttttcggctacaaagtttgcttcattcatcacccagtattccaca |
| | | ggacaagtgagcgtggagattgaatgggagctgcagaaagaaaacagcaaac |
| | | gctggaatcccgaagtgcagtatacatctaactatgcaaaatctgccaacgt |
| | | tgatttcactgtggacaacaatggactttatactgagcctcgccccattggc |
| | | acccgttacctcacccgtcccctgtaa |
| AAV9 | 21 | MAADGYLPDW LEDNLSEGIR EWWALKPGAP QPKANQQHOD |
| VP1 | | NARGLVLPGY KYLGPGNGLD KGEPVNAADA AALEHDKAYD |
| | | QQLKAGDNPY LKYNHADAEF QERLKEDTSF GGNLGRAVFQ |
| | | AKKRLLEPLG LVEEAAKTAP GKKRPVEQSP QEPDSSAGIG |
| | | KSGAQPAKKR LNFGQTGDTE SVPDPQPIGE PPAAPSGVGS |
| | | LTMASGGGAP VADNNEGADG VGSSSGNWHC DSQWLGDRVI |
| | | TTSTRTWALP TYNNHLYKQI SNSTSGGSSN DNAYFGYSTP |
| | | WGYFDENRFH CHESPRDWQR LINNNWGFRP KRLNFKLFNI |
| | | QVKEVTDNNG VKTIANNLTS TVQVFTDSDY QLPYVLGSAH |
| | | EGCLPPFPAD VFMIPQYGYL TLNDGSQAVG RSSFYCLEYF |
| | | PSQMLRTGNN FQFSYEFENV PFHSSYAHSQ SLDRLMNPLI |
| | | DQYLYYLSKT INGSGQNQQT LKFSVAGPSN MAVQGRNYIP |
| | | GPSYRQQRVS TTVTQNNNSE FAWPGASSWA LNGRNSLMNP |
| | | GPAMASHKEG EDRFFPLSGS LIFGKQGTGR DNVDADKVMI |
| | | TNEEEIKTTN PVATESYGQV ATNHQSAQAQ AQTGWVQNQG |
| | | ILPGMVWQDR DVYLQGPIWA KIPHTDGNFH PSPLMGGFGM |
| | | KHPPPQILIK NTPVPADPPT AFNKDKLNSF ITQYSTGQVS |
| | | VEIEWELQKE NSKRWNPEIQ YTSNYYKSNN VEFAVNTEGV |
| | | YSEPRPIGTR YLTRNL |
| AAV9 | 22 | atggctgccgatggttatcttccagattggctcgaggacaaccttagtgaag |
| VP1 | | gaattcgcgagtggtgggctttgaaacctggagcccctcaacccaaggcaaa |
| coding | | tcaacaacatcaagacaacgctcgaggtcttgtgcttccgggttacaaatac |
| sequence | | cttggacccggcaacggactcgacaagggggagccggtcaacgcagcagacg |
| | | cggcggccctcgagcacgacaaggcctacgaccagcagctcaaggccggaga |
| | | caacccgtacctcaagtacaaccacgccgacgccgagttccaggagcggctc |
| | | aaagaagatacgtcttttgggggcaacctcgggcgagcagtcttccaggcca |
| | | aaaagaggcttcttgaacctcttggtctggttgaggaagcggctaagacggc |
| | | tcctggaaagaagaggcctgtagagcagtctcctcaggaaccggactcctcc |
| | | gcgggtattggcaaatcgggtgcacagcccgctaaaaagagactcaatttcg |
| | | gtcagactggcgacacagagtcagtcccagaccctcaaccaatcggagaacc |
| | | tcccgcagccccctcaggtgtgggatctcttacaatggcttcaggtggtggc |
| | | gcaccagtggcagacaataacgaaggtgccgatggagtgggtagttcctcgg |
| | | gaaattggcattgcgattcccaatggctgggggacagagtcatcaccaccag |
| | | cacccgaacctgggccctgcccacctacaacaatcacctctacaagcaaatc |
| | | tccaacagcacatctggaggatcttcaaatgacaacgcctacttcggctaca |
| | | gcaccccctgggggtattttgacttcaacagattccactgccacttctcacc |
| | | acgtgactggcagcgactcatcaacaacaactggggattccggcctaagcga |
| | | ctcaacttcaagctcttcaacattcaggtcaaagaggttacggacaacaatg |
| | | gagtcaagaccatcgccaataaccttaccagcacggtccaggtcttcacgga |
| | | ctcagactatcagctcccgtacgtgctcgggtcggctcacgagggctgcctc |
| | | ccgccgttcccagcggacgttttcatgattcctcagtacgggtatctgacgc |
| | | ttaatgatggaagccaggccgtgggtcgttcgtccttttactgcctggaata |
| | | tttcccgtcgcaaatgctaagaacgggtaacaacttccagttcagctacgag |
| | | tttgagaacgtacctttccatagcagctacgctcacagccaaagcctggacc |
| | | gactaatgaatccactcatcgaccaatacttgtactatctctcaaagactat |
| | | taacggttctggacagaatcaacaaacgctaaaattcagtgtggccggaccc |
| | | agcaacatggctgtccagggaagaaactacatacctggacccagctaccgac |
| | | aacaacgtgtctcaaccactgtgactcaaaacaacaacagcgaatttgcttg |
| | | gcctggagcttcttcttgggctctcaatggacgtaatagcttgatgaatcct |
| | | ggacctgctatggccagccacaaagaaggagaggaccgtttctttcctttgt |
| | | ctggatctttaatttttggcaaacaaggaactggaagagacaacgtggatgc |
| | | ggacaaagtcatgataaccaacgaagaagaaattaaaactactaacccggta |
| | | gcaacggagtcctatggacaagtggccacaaaccaccagagtgcccaagcac |
| | | aggcgcagaccggctgggttcaaaaccaaggaatacttccgggtatggtttg |
| | | gcaggacagagatgtgtacctgcaaggacccatttgggccaaaattcctcac |
| | | acggacggcaactttcacccttctccgctgatgggagggtttggaatgaagc |
| | | acccgcctcctcagatcctcatcaaaaacacacctgtacctgcggatcctcc |
| | | aacggccttcaacaaggacaagctgaactctttcatcacccagtattctact |
| | | ggccaagtcagcgtggagatcgagtgggagctgcagaaggaaaacagcaagc |
| | | gctggaacccggagatccagtacacttccaactattacaagtctaataatgt |
| | | tgaatttgctgttaatactgaaggtgtatatagtgaaccccgccccattggc |
| | | accagatacctgactcgtaatctgtaa |
| AAV8 | 23 | MAADGYLPDWLEDNLSEGIREWWALKPGAPKPKANQQKQDDGRGLVLPGYKY |
| VPI | | LGPENGLDKGEPVNAADAAALEHDKAYDQQLQAGDNPYLRYNHADAEFQERL |
| (GenBank | | QEDTSFGGNLGRAVFQAKKRVLEPLGLVEEGAKTAPGKKRPVEPSPQRSPDS |
| AAN03857) | | STGIGKKGQQPARKRLNFGQTGDSESVPDPQPLGEPPAAPSGVGPNTMAAGG |
| | | GAPMADNNEGADGVGSSSGNWHCDSTWLGDRVITTSTRTWALPTYNNHLYKQ |
| | | ISNGTSGGATNDNTYFGYSTPWGYFDFNRFHCHFSPRDWORLINNNWGFRPK |
| | | RLSFKLFNIQVKEVTQNEGTKTIANNLTSTIQVFTDSEYQLPYVLGSAHQGC |
| | | LPPFPADVFMIPQYGYLTLNNGSQAVGRSSFYCLEYFPSQMLRTGNNFQFTY |
| | | TFEDVPFHSSYAHSQSLDRLMNPLIDQYLYYLSRTQTTGGTANTQTLGFSQG |
| | | GPNTMANQAKNWLPGPCYRQQRVSTTTGQNNNSNFAWTAGTKYHLNGRNSLA |
| | | NPGIAMATHKDDEERFFPSNGILIFGKQNAARDNADYSDVMLTSEEEIKTTN |
| | | PVATEEYGIVADNLQQONTAPQIGTVNSQGALPGMVWQNRDVYLQGPIWAKI |
| | | PHTDGNFHPSPLMGGFGLKHPPPQILIKNTPVPADPPTTFNQSKLNSFITQY |
| | | STGQVSVEIEWELQKENSKRWNPEIQYTSNYYKSTSVDFAVNTEGVYSEPRP |
| | | IGTRYLTRNL |
| AAV8 | 24 | CAGAGAGGGAGTGGCCAACTCCATCACTAGGGGTAGCGCGAAGCGCCTCCCACGCTGC |
| VP1 | | CGCGTCAGCGCTGACGTAAATTACGTCATAGGGGAGTGGTCCTGTATTAGCTGTCACG |
| coding | | TGAGTGCTTTTGCGGCATTTTGCGACACCACGTGGCCATTTGAGGTATATATGGCCGA |
| sequence | | GTGAGCGAGCAGGATCTCCATTTTGACCGCGAAATTTGAACGAGCAGCAGCCATGCCG |
| AF513852.1 | | GGCTTCTACGAGATCGTGATCAAGGTGCCGAGCGACCTGGACGAGCACCTGCCGGGCA |
| | | TTTCTGACTCGTTTGTGAACTGGGTGGCCGAGAAGGAATGGGAGCTGCCCCCGGATTC |
| | | TGACATGGATCGGAATCTGATCGAGCAGGCACCCCTGACCGTGGCCGAGAAGCTGCAG |
| | | CGCGACTTCCTGGTCCAATGGCGCCGCGTGAGTAAGGCCCCGGAGGCCCTCTTCTTTG |
| | | TTCAGTTCGAGAAGGGCGAGAGCTACTTTCACCTGCACGTTCTGGTCGAGACCACGGG |
| | | GGTCAAGTCCATGGTGCTAGGCCGCTTCCTGAGTCAGATTCGGGAAAAGCTTGGTCCA |
| | | GACCATCTACCCGCGGGGTCGAGCCCCACCTTGCCCAACTGGTTCGCGGTGACCAAAG |
| | | ACGCGGTAATGGCGCCGGCGGGGGGGAACAAGGTGGTGGACGAGTGCTACATCCCCAA |
| | | CTACCTCCTGCCCAAGACTCAGCCCGAGCTGCAGTGGGCGTGGACTAACATGGAGGAG |
| | | TATATAAGCGCGTGCTTGAACCTGGCCGAGCGCAAACGGCTCGTGGCGCAGCACCTGA |
| | | CCCACGTCAGCCAGACGCAGGAGCAGAACAAGGAGAATCTGAACCCCAATTCTGACGC |
| | | GCCCGTGATCAGGTCAAAAACCTCCGCGCGCTATATGGAGCTGGTCGGGTGGCTGGTG |
| | | GACCGGGGCATCACCTCCGAGAAGCAGTGGATCCAGGAGGACCAGGCCTCGTACATCT |
| | | CCTTCAACGCCGCCTCCAACTCGCGGTCCCAGATCAAGGCCGCGCTGGACAATGCCGG |
| | | CAAGATCATGGCGCTGACCAAATCCGCGCCCGACTACCTGGTGGGGCCCTCGCTGCCC |
| | | GCGGACATTACCCAGAACCGCATCTACCGCATCCTCGCTCTCAACGGCTACGACCCTG |
| | | CCTACGCCGGCTCCGTCTTTCTCGGCTGGGCTCAGAAAAAGTTCGGGAAACGCAACAC |
| | | CATCTGGCTGTTTGGACCCGCCACCACCGGCAAGACCAACATTGCGGAAGCCATCGCC |
| | | CACGCCGTGCCCTTCTACGGCTGCGTCAACTGGACCAATGAGAACTTTCCCTTCAATG |
| | | ATTGCGTCGACAAGATGGTGATCTGGTGGGAGGAGGGCAAGATGACGGCCAAGGTCGT |
| | | GGAGTCCGCCAAGGCCATTCTCGGCGGCAGCAAGGTGCGCGTGGACCAAAAGTGCAAG |
| | | TCGTCCGCCCAGATCGACCCCACCCCCGTGATCGTCACCTCCAACACCAACATGTGCG |
| | | CCGTGATTGACGGGAACAGCACCACCTTCGAGCACCAGCAGCCTCTCCAGGACCGGAT |
| | | GTTTAAGTTCGAACTCACCCGCCGTCTGGAGCACGACTTTGGCAAGGTGACAAAGCAG |
| | | GAAGTCAAAGAGTTCTTCCGCTGGGCCAGTGATCACGTGACCGAGGTGGCGCATGAGT |
| | | TTTACGTCAGAAAGGGCGGAGCCAGCAAAAGACCCGCCCCCGATGACGCGGATAAAAG |
| | | CGAGCCCAAGCGGGCCTGCCCCTCAGTCGCGGATCCATCGACGTCAGACGCGGAAGGA |
| | | GCTCCGGTGGACTTTGCCGACAGGTACCAAAACAAATGTTCTCGTCACGCGGGCATGC |
| | | TTCAGATGCTGTTTCCCTGCAAAACGTGCGAGAGAATGAATCAGAATTTCAACATTTG |
| | | CTTCACACACGGGGTCAGAGACTGCTCAGAGTGTTTCCCCGGCGTGTCAGAATCTCAA |
| | | CCGGTCGTCAGAAAGAGGACGTATCGGAAACTCTGTGCGATTCATCATCTGCTGGGGC |
| | | GGGCTCCCGAGATTGCTTGCTCGGCCTGCGATCTGGTCAACGTGGACCTGGATGACTG |
| | | TGTTTCTGAGCAATAAATGACTTAAACCAGGTATGGCTGCCGATGGTTATCTTCCAGA |
| | | TTGGCTCGAGGACAACCTCTCTGAGGGCATTCGCGAGTGGTGGGCGCTGAAACCTGGA |
| | | GCCCCGAAGCCCAAAGCCAACCAGCAAAAGCAGGACGACGGCCGGGGTCTGGTGCTTC |
| | | CTGGCTACAAGTACCTCGGACCCTTCAACGGACTCGACAAGGGGGAGCCCGTCAACGC |
| | | GGCGGACGCAGCGGCCCTCGAGCACGACAAGGCCTACGACCAGCAGCTGCAGGCGGGT |
| | | GACAATCCGTACCTGCGGTATAACCACGCCGACGCCGAGTTTCAGGAGCGTCTGCAAG |
| | | AAGATACGTCTTTTGGGGGCAACCTCGGGCGAGCAGTCTTCCAGGCCAAGAAGCGGGT |
| | | TCTCGAACCTCTCGGTCTGGTTGAGGAAGGCGCTAAGACGGCTCCTGGAAAGAAGAGA |
| | | CCGGTAGAGCCATCACCCCAGCGTTCTCCAGACTCCTCTACGGGCATCGGCAAGAAAG |
| | | GCCAACAGCCCGCCAGAAAAAGACTCAATTTTGGTCAGACTGGCGACTCAGAGTCAGT |
| | | TCCAGACCCTCAACCTCTCGGAGAACCTCCAGCAGCGCCCTCTGGTGTGGGACCTAAT |
| | | ACAATGGCTGCAGGCGGTGGCGCACCAATGGCAGACAATAACGAAGGCGCCGACGGAG |
| | | TGGGTAGTTCCTCGGGAAATTGGCATTGCGATTCCACATGGCTGGGCGACAGAGTCAT |
| | | CACCACCAGCACCCGAACCTGGGCCCTGCCCACCTACAACAACCACCTCTACAAGCAA |
| | | ATCTCCAACGGGACATCGGGAGGAGCCACCAACGACAACACCTACTTCGGCTACAGCA |
| | | CCCCCTGGGGGTATTTTGACTTTAACAGATTCCACTGCCACTTTTCACCACGTGACTG |
| | | GCAGCGACTCATCAACAACAACTGGGGATTCCGGCCCAAGAGACTCAGCTTCAAGCTC |
| | | TTCAACATCCAGGTCAAGGAGGTCACGCAGAATGAAGGCACCAAGACCATCGCCAATA |
| | | ACCTCACCAGCACCATCCAGGTGTTTACGGACTCGGAGTACCAGCTGCCGTACGTTCT |
| | | CGGCTCTGCCCACCAGGGCTGCCTGCCTCCGTTCCCGGCGGACGTGTTCATGATTCCC |
| | | CAGTACGGCTACCTAACACTCAACAACGGTAGTCAGGCCGTGGGACGCTCCTCCTTCT |
| | | ACTGCCTGGAATACTTTCCTTCGCAGATGCTGAGAACCGGCAACAACTTCCAGTTTAC |
| | | TTACACCTTCGAGGACGTGCCTTTCCACAGCAGCTACGCCCACAGCCAGAGCTTGGAC |
| | | CGGCTGATGAATCCTCTGATTGACCAGTACCTGTACTACTTGTCTCGGACTCAAACAA |
| | | CAGGAGGCACGGCAAATACGCAGACTCTGGGCTTCAGCCAAGGTGGGCCTAATACAAT |
| | | GGCCAATCAGGCAAAGAACTGGCTGCCAGGACCCTGTTACCGCCAACAACGCGTCTCA |
| | | ACGACAACCGGGCAAAACAACAATAGCAACTTTGCCTGGACTGCTGGGACCAAATACC |
| | | ATCTGAATGGAAGAAATTCATTGGCTAATCCTGGCATCGCTATGGCAACACACAAAGA |
| | | CGACGAGGAGCGTTTTTTTCCCAGTAACGGGATCCTGATTTTTGGCAAACAAAATGCT |
| | | GCCAGAGACAATGCGGATTACAGCGATGTCATGCTCACCAGCGAGGAAGAAATCAAAA |
| | | CCACTAACCCTGTGGCTACAGAGGAATACGGTATCGTGGCAGATAACTTGCAGCAGCA |
| | | AAACACGGCTCCTCAAATTGGAACTGTCAACAGCCAGGGGGCCTTACCCGGTATGGTC |
| | | TGGCAGAACCGGGACGTGTACCTGCAGGGTCCCATCTGGGCCAAGATTCCTCACACGG |
| | | ACGGCAACTTCCACCCGTCTCCGCTGATGGGCGGCTTTGGCCTGAAACATCCTCCGCC |
| | | TCAGATCCTGATCAAGAACACGCCTGTACCTGCGGATCCTCCGACCACCTTCAACCAG |
| | | TCAAAGCTGAACTCTTTCATCACGCAATACAGCACCGGACAGGTCAGCGTGGAAATTG |
| | | AATGGGAGCTGCAGAAGGAAAACAGCAAGCGCTGGAACCCCGAGATCCAGTACACCTC |
| | | CAACTACTACAAATCTACAAGTGTGGACTTTGCTGTTAATACAGAAGGCGTGTACTCT |
| | | GAACCCCGCCCCATTGGCACCCGTTACCTCACCCGTAATCTGTAATTGCCTGTTAATC |
| | | AATAAACCGGTTGATTCGTTTCAGTTGAACTTTGGTCTCTGCG |

### Example 1

The purpose of this study is to evaluate the therapeutic efficacy of the AAV9-based Pax4 gene delivery vectors in streptozotocin (STZ)-induced T1D mouse models. To achieve efficient Pax4 gene delivery in vivo, AAV9-based Pax4 gene delivery vectors are generated in which human Pax4 cDNA is controlled by either chicken beta-actin core promoter region with cytomegalovirus (CMV) early enhancer element or a CAG promoter (SEQ ID NO: 4), or rat insulin promoter (RIP) (SEQ ID NO: 6), or mouse glucagon promoter (MGP) (SEQ ID NO: 5). While the CAG promoter provides strong and ubiquitous gene expression, RIP and MGP promoters provide β-cell-specific and α-cell-specific gene expression, respectively. The efficacy of AAV9-based Pax4 gene delivery will be evaluated by the expected improvements in T1D parameters in STZ mouse model.

### Experimental Design

The experiment will use 6 experimental groups of mice (n≥5 mice/group, females):
1. Normal mice as control
2. Untreated T1D mice as control
3. AAV9.CAG-eGFP treated T1D mice (control treatment)
4. AAV9.CAG-Pax4 treated T1D mice
5. AAV9.RIP-Pax4 treated T1D mice
6. AAV9.MGP-Pax4 treated T1D mice.

C57BL/6 mice (Jackson Laboratories), 6-8 weeks old will be housed for at least 7 days and then will be injected with 65 mg/Kg bodyweight of STZ daily, for 5 consecutive days, via i.p. injections. Blood glucose will be measured using ALPHA TRAK2 blood glucose monitoring meter and test strips, every other day, for the next 2 weeks. 1 to 2 weeks after the last STZ injection, once the blood glucose of the mouse is >300 mg/dL, the rAAV vectors will be introduced by intra-bile ductal injection, at a dose of 3.0E11 vg/mouse in a total volume of 75 l. The mice will be observed 3 times a day for the first 2 days after the surgery, twice a day for one week, and then daily for another week. Blood Glucose monitoring will be carried out 3 times a week for the first 2 weeks after surgery, and then twice a week for the next 10 weeks. All measurements will be taken at approximately the same time of day. Glucose tolerance test (GTT) will be performed at weeks 2, 6, and 10. Glucose-stimulated insulin secretion (GSIS) assay will be performed at weeks 4, 8, and 12. At 12 weeks after surgery, mice will be sacrificed by cardiac puncture under anesthesia, collecting blood and harvesting the pancreas, liver, spleen, intestine and kidney for biochemical and immunohistochemical assays. Hormone levels (insulin, glucagon, C-peptide) in blood samples will be measures using ELISA kits. Immunohistochemistry assessment of tissue slices (to examine alpha cells, beta cells, and to examine Pax4 expression).

### Statistical Analyses:

The statistical analyses will be performed using the GraphPad Prism 9 software to compare different treatment groups for their blood glucose, GTT data, and GSIS data. When analyzing glucose levels among multiple groups over the time (two parameters, as in GTT and GSIS), two-way ANOVA will be performed to determine the significance of their differences. For each data point (at a fixed time point), one-way ANOVA will be performed to determine the significance of differences among 3 or more groups. If comparison of two groups is needed, Student's t-test will be performed. The data will be expressed as Mean±SEM unless indicated otherwise. P<0.05 is considered statistically significant.

### Example 2—Preclinical Pilot Study of AAV9-Mediated Pax4 Gene Delivery into Pancreas for Treatment of Type 1 Diabetes

AAV9-based vectors were developed for Pax4 gene delivery in vivo. AAV9 was chosen because it has been shown to mediate significant gene delivery into pancreas following systemic or intra-bile ductal (IBD) administration, and AAV9-based vectors are being used in clinical trials. For this study, three types of AAV9.Pax4 vectors were generated to deliver human Pax4 cDNA: i) AAV9.CAG-Pax4 in which Pax4 expression is driven by the ubiquitously active CAG promoter; ii) AAV9.MGP-Pax4 in which Pax4 is driven by mouse glucagon promoter; and iii) AAV9.RIP-Pax4 in which Pax4 is driven by rat insulin promoter.

### Summary of Study

The objective of this pre-clinical study was to evaluate the therapeutic effects of these AAV9.Pax4 vectors in streptozotocin (STZ)-induced T1D mouse models. The STZ-T1D models were established in C57BL/6 mice by multiple-low doses of STZ injection (5 daily doses of 65 mg/kg bodyweight). When their non-fasting blood glucose (BG) reached >300 mg/dL, they were treated with a single dose of 3×10¹¹ vg/mouse of the AAV9.Pax4 vectors or control (AAV9.CAG-GFP) via IBD injection, which allowed direct vector administration into pancreas through the pancreatic ductal system. PBS (phosphate-buffered saline) was injected into the T1D mice as vehicle control. In addition, 5 mice from the same batch were maintained as normal controls (that is, they were not subjected to STZ treatment or IBD injection). Non-fasting blood glucose was monitored 2-3 times per week in STZ mice and weekly in normal mice for approximately 3 months. Intraperitoneal glucose tolerance test (GTT) was performed about 8 weeks after vector administration. Glucose stimulated insulin secretion (GSIS) assay was performed about 10 weeks after vector administration. At ˜3 month after the treatment, the mice were sacrificed. Their blood was collected for hormone (insulin, glucagon, C-peptide) measurement by corresponding ELISA kits, and the pancreas harvested and processed for immunohistochemistry analysis of islet α and β cells, as well as Pax4 (or GFP) transgene expression.

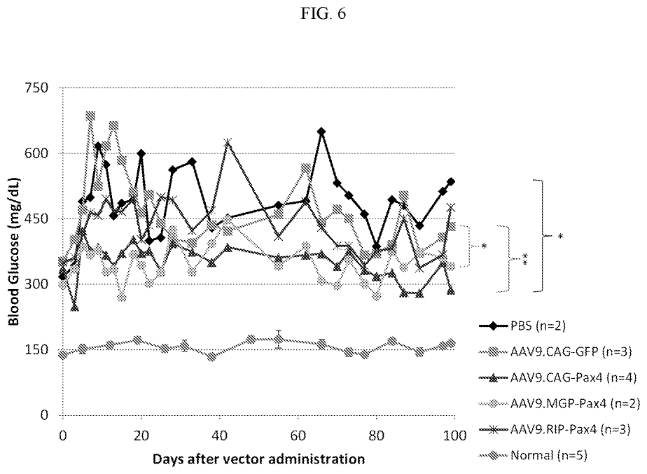

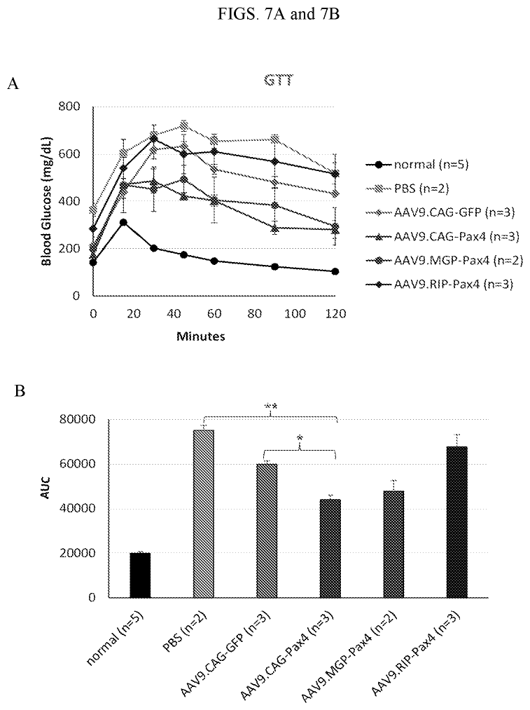

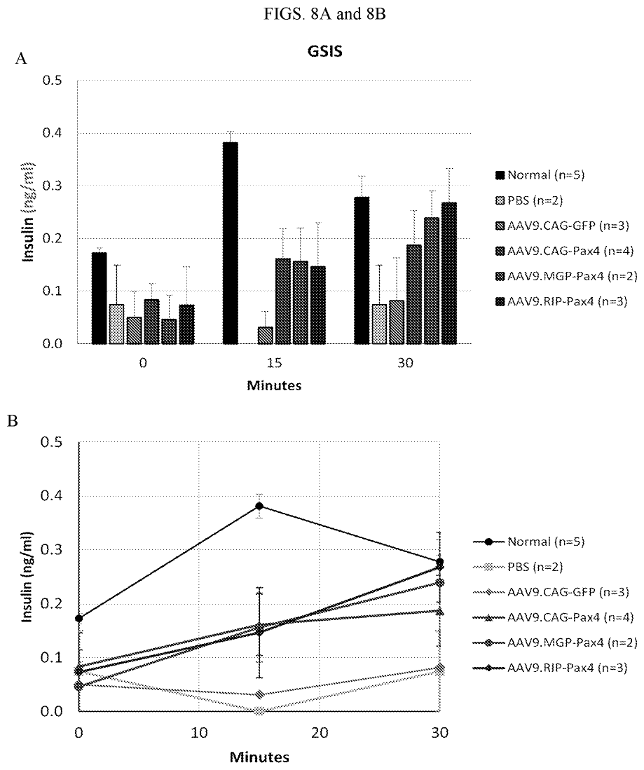

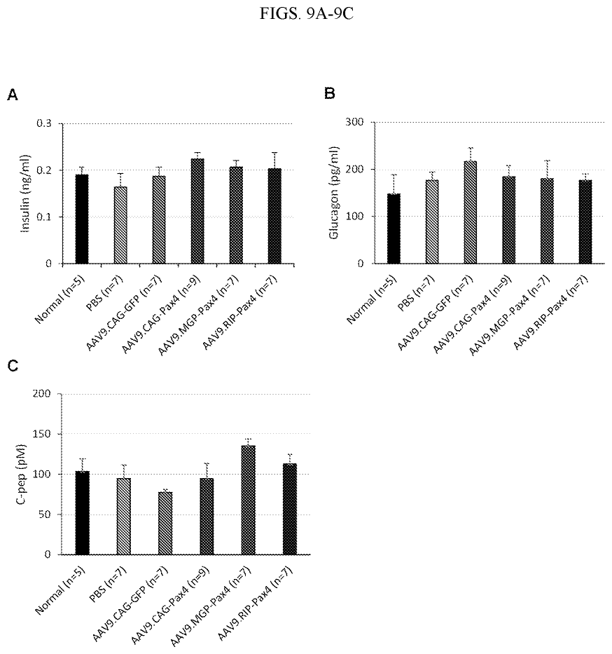

The STZ-treated mice developed hyperglycemia following STZ treatment at a significantly faster than expected rate compared to previous STZ dosing experience and, thus, the STZ-treated mice that developed hyperglycemia faster than expected were treated with the rAAV vectors as early as 3 days after the last STZ dose (at least 4 days earlier than originally expected). The effects of these vector treatments less than 7 days after the last STZ dose could therefore be complicated by the presence of on-going STZ-induced β cell death. Accordingly, data from the mice that developed hyperglycemia after 1 week of last STZ dose, and, thus, treated later were analyzed separately from the data generated from the mice that developed hyperglycemia before 1 week after last STZ dose. The results from these mice, treated 1 week after last STZ dose, show that AAV9.Pax4 vectors showed signs of therapeutic benefit compared to the control treatments. Specifically, AAV9.CAG-Pax4 and AAV9.MGP-Pax4-treated T1D mice showed significantly lower non-fasting BG than the control-treated T1D mice, despite the small sample size (n=2-4) (FIG. 6). In evaluated animals, AAV9.CAG-Pax4-treated mice showed significant improvement in GTT (FIGS. 7A and 7B), and all T iD model mice treated with one of the 3 AAV9.Pax4 vectors showed tendency of higher insulin secretion than the control treatments in response to glucose challenge as assessed by GSIS assay (FIGS. 8A and 8B). Moreover, circulating insulin and C-peptide in AAV9.Pax4-treated mice were significantly higher than the control PBS or AAV9.CAG-GFP-treated mice (FIGS. 9A and 9C), suggesting Pax4 treatment improved β cell mass/function. On the other hand, circulating glucagon didn't show significant differences among the treatment groups (FIG. 9B), suggesting Pax4 gene therapy did not result in glucagon deficiency. Finally, IHC staining of the pancreatic slices showed Pax4 and GFP expression, confirming AAV9-mediated transgene expression is long-lasting (data not shown).

In summary, despite the non-optimal T1D models, the observations that a single dose of the AAV9.Pax4 vectors could improve β cell function and ameliorate hyperglycemia support the continued development of AAV9-based Pax4 gene therapy as a means to treat T1D.

### INTRODUCTION

3 AAV9-based human Pax4 gene delivery vectors were generated for testing:
i) AAV9.CAG-Pax4 in which Pax4 expression is driven by chicken beta-actin core promoter region with cytomegalovirus (CMV) early enhancer element (CAG) promoter. The ubiquitous activity of the CAG promoter will drive Pax4 expression in both α and β cells, having the potential to both induce transdifferentiation in α cells and promoting survival of remaining and newly transdifferentiated β cells. However, the strong ubiquitous promoter activity will also enable Pax4 expression in non-islet and non-pancreatic transduced cells, resulting in off-target transgene expression.
ii) AAV9.MGP-Pax4 in which Pax4 is driven by mouse glucagon promoter (MGP) which drives cell specific transgene expression in pancreatic islet α cells, but is expected to have minimal promoter activity in glucagon negative pancreatic islet β cells.
iii) AAV9.RIP-Pax4 in which Pax4 is driven by rat insulin promoter (RIP) which drives cell specific transgene expression in pancreatic islet β cells, but is expected to have minimal promoter activity in insulin negative pancreatic islet α cells.

### Objective

The objective of this study was to evaluate the therapeutic efficacy of the AAV9-based Pax4 gene delivery vectors in streptozotocin (STZ)-induced TiD mouse models following direct vector administration into pancreas via intra-bile ductal (IBD) injection. Three vectors, AAV9.CAG-Pax4, AAV9.MGP-Pax4 and AAV9.RIP-Pax4, were tested.

### Materials and Methods

rAAV constructs and concentrations used in this study are listed in Tables 2 and 3.

TABLE 2
1^{st} batch Constructs

| | Nominal | |
|---|---|---|
| Name | Concentration | Excipients |
| AAV9.CAG-Pax4 | 1.0749E+13 vg/ml | PBS and 5% Glycerol |
| AAV9.MGP-Pax4 | 7.7418E+12 vg/ml | PBS and 5% Glycerol |
| AAV9.RIP-Pax4 | 1.2243E+13 vg/ml | PBS and 5% Glycerol |

TABLE 3
2^{nd} batch Constructs

| | Nominal | |
|---|---|---|
| Name | Concentration | Excipients |
| AAV9.CAG-Pax4 | 7.8527E+12 vg/ml | PBS and 5% Glycerol |
| AAV9.MGP-Pax4 | 5.9939E+12 vg/ml | PBS and 5% Glycerol |
| AAV9.RIP-Pax4 | 8.6333E+12 vg/ml | PBS and 5% Glycerol |

Vehicle controls used in this study are listed in Table 4.

TABLE 4
Vehicle controls:

| | Nominal | |
|---|---|---|
| Name | Concentration | Excipients |
| PBS 1X (Phosphate- | N/A | 154 mM NaCl, 5.6 mM |
| Buffered Saline) | | Na2HPO4 and 1 mM |
| | | KH2PO4, pH 7.4 |
| AAV9.CAG-GFP | 1.5883E+13 vg/ml | PBS and 5% Glycerol |

### Materials/Supplies:

Animals: C57BL/6 mice, female, ˜11 weeks old, from Charles River (Stock #027). Weight at randomization: 19.1 g±0.15 g.

Glucose measurement: AlphaTRAK2 blood glucose monitoring system (Zoetis), including glucometer and blood glucose test strips.

Mouse ultrasensitive insulin ELISA kits: cat #80-INSMSU-E01, from ALPCO.

Glucagon ELISA kit: cat #48-GLUHU-E01, from ALPCO.

Mouse C-peptide ELISA kit: cat #80-CPTMS-E01, from ALPCO.

Antibodies used in the study are listed in Table 5.

TABLE 5
List of antibodies.

| | | | Dilution used |
|---|---|---|---|
| Antibody | Vendor | Catalog # | (for IHC) |
| Insulin polyclonal antibody | Invitrogen | PA1-26938 | 1:75 |
| (Guinea pig) | | | |
| Glucagon monoclonal | Sigma-Aldrich | G2654 | 1:500 |
| antibody (Mouse) | | | |
| Glucagon polyclonal | Cell Signaling | 2760 | 1:100 |
| antibody (Rabbit) | Technology | | |
| GFP monoclonal antibody | Invitrogen | 3E6 | 1:500 |
| (mouse) | | | |
| Pax4 polyclonal antibody | Invitrogen | PA1-108 | 1:500 |
| (rabbit) | | | |

### Experimental Design

There were 6 groups (n≥5 mice/group, females) in the study:
1) Normal, untreated mice (as control)
2) PBS-treated STZ-T1D mice (as vehicle control)
3) AAV9.CAG-GFP treated STZ-T1D mice (as vector control)
4) AAV9.CAG-Pax4 treated STZ-T1D mice
5) AAV9.MGP-Pax4 treated STZ-T1D mice
6) AAV9.RIP-Pax4 treated STZ-T1D mice

The procedure is detailed in Table 6.

TABLE 6
Study procedure:

| | |
|---|---|
| 1 | C57BL/6 mice purchased from Charles River, 8 weeks old, n = 50 mice. |
| 2 | When the mice were 11 weeks old, they were injected with 65 mg/Kg bodyweight of STZ |
| | daily, for 5 consecutive days, via i.p. injections. |
| 3 | Their blood glucose is measured using ALPHA TRAK2 blood glucose monitoring |
| | meter and test strips, every other day. |
| 4 | Performed intra-bile ductal (IBD) injection when their blood glucose was |
| | >300 mg/dL, which started 2 days after the last STZ injection. |
| | The mice were randomly assigned into each treatment group based on their blood |
| | glucose levels. For each mouse, 3 × 10¹¹ vg of AAV9 vector diluted into 50 μl (total |
| | volume) with PBS was injected into the pancreas via IBD injection. |
| 5 | Post-operative care. Buprenorphine (0.1 mg/Kg) were given every 12 hours for 48 |
| | hours after the surgery. The mice were observed 3 times a day for the first 2 days |
| | after the surgery, twice a day for one week, and then daily for another week. |
| 6 | Blood Glucose monitored 3 times a week for the first 2 weeks after surgery, twice a |
| | week for the next 10 weeks. All measurements were taken between 1-2 pm. |
| 7 | Glucose tolerance test (GTT) were performed at ~8 weeks after IBD injection. |
| 8 | Glucose-stimulated insulin secretion (GSIS) assay were performed at ~10 weeks |
| | after IBD injection. |
| 9 | At ~3 months after surgery, blood (1.2 ml) was collected from the mice via cardiac |
| | puncture under anesthesia. The procedure resulted in the death of the mice. |
| 10 | Harvested the pancreas, liver, kidney, and spleen, and preserved them in 10% |
| | formalin solution for future IHC assays. |
| 11 | Measured hormones (insulin, glucagon, C-peptide) in blood samples using ELISA |
| | kits from ALPCO. |
| 12 | Pancreatic tissues were processed for IHC assessment (to examine a and β cell |
| | condition, and to examine Pax4 (or GFP) expression. |

The mice were subjected to 5 daily doses of 65 mg/kg bodyweight via i.p. injections. Animals were randomly assigned to treatments based on theirblood glucose levels. For each mouse, 3×10¹¹ VG of AAV9 vector diluted into 50 μl (total volume) with PBS was injected into the pancreas via IBD injection. The details of each treatment group are listed in Table 7. The gray shade marks the mice that developed hyperglycemia after 1 week of the last STZ dose and were utilized for further hormone and biomarker analysis.

TABLE 7
Details of Body Weight

| | | group | Blood Glucose | |
|---|---|---|---|---|
| Treatment | Mouse | Body Weight | (mg/dL) at | |
| Group | # | (g) at surgery | surgery | Notes |
| PBS | 2 | 18.6 | 277 | |
| | 3 | 18.1 | 271 | |
| | 10 | 19.1 | 337 | Euthanized due to surgical issue |
| | 14 | 18.8 | 289 | |
| | 21 | 19.6 | 370 | |
| | 23 | 18.9 | 318 | |
| | 17 | 19.3 | 347 | |
| | 20 | 18.6 | 288 | |
| AAV9.CAG- | 1 | 19.0 | 331 | |
| GFP | 6 | 16.7 | 310 | |
| | 34 | 17.9 | 314 | |
| | 36 | 19.4 | 306 | |
| | 7 | 18.2 | 284 | |
| | 15 | 19.7 | 315 | |
| | 22 | 19.2 | 460 | |
| AAV9.CAG- | 19 | 18.2 | 324 | 1^{st} batch vector |
| Pax4 | 26 | 18.9 | 318 | |
| | 28 | 18.0 | 317 | |
| | 29 | 20.2 | 387 | |
| | 40 | 20.0 | 378 | |
| | 12* | 18.1 | 359 | 2^{nd} batch vector |
| | 16* | 19.4 | 313 | (* marked mice) |
| | 18* | 20.1 | 303 | |
| | 38* | 19.6 | 365 | |
| AAV9.MGP- | 31 | 18.2 | 310 | 1^{st} batch vector |
| Pax4 | 32 | 18.5 | 317 | |
| | 33 | 19.8 | 314 | |
| | 35 | 17.0 | 343 | |
| | 39 | 18.3 | 336 | |
| | 5* | 20.0 | 278 | 2^{nd} batch vector |
| | 8* | 19.8 | 320 | (* marked mice) |
| AAV9.RIP- | 27 | 17.7 | 306 | 1^{st} batch vector |
| Pax4 | 30 | 18.7 | 280 | |
| | 37 | 19.0 | 360 | |
| | 45 | 19.0 | 302 | |
| | 42* | 19.3 | 434 | 2^{nd} batch vector |
| | 43* | 20.9 | 290 | (* marked mice) |
| | 25* | 20.3 | 324 | |
| Normal mice | 46 | 19.2 | 122 | |
| | 47 | 18.5 | 131 | |
| | 48 | 19.7 | 145 | |
| | 49 | 21.0 | 152 | |
| | 50 | 22.6 | 140 | |

### Bodyweight and Clinical Observations

Body weight was measured whenever blood glucose was measured, which is 2-3 times/week for about 3 months.

Blood glucose (non-fasting) was monitored 2-3 times per week for ˜3 months.

Glucose tolerance test (GTT) was performed ˜8 weeks after vector dosing.

Glucose stimulated insulin secretion (GSIS) assay was performed ˜10 weeks after vector dosing.

### Laboratory Procedures

T1D induction with multiple low doses of STZ. T1D induction was performed as described previously (Zhang et al., (2016), Bone et al.) Specifically, female C57Bl/6 mice (from Charles River), ˜11 weeks old, were fasted for 5 hours and then injected with 65 mg/kg bodyweight of STZ via intra-peritoneal (i.p.) injections for 5 consecutive days. Blood glucose was monitored with AlphaTRAK2 Blood Glucose monitoring system every other day. Treatment with test articles or controls began when blood glucose (non-fasting) reached >300 mg/dL.

Intra-bile ductal (IBD) injection. The procedure was performed as described in previous studies (Zhang et al., (2016), Bone et al.), with modifications that included a smaller injection volume and clamping of duodenum. Specifically, prior to surgery, all surgical instruments were sterilized by autoclave. At the time of surgery, the workstation was thoroughly cleaned with 70% ethanol. In-between surgeries, the instruments were sterilized with a dry bead sterilizer. The surgery was performed under a Leica M80 stereo microscope coupled with Leica IC90 E camera (microinjection system). 70% ethanol spray was used to make sure the work area was clean and sterile. For the surgery, the mouse was anesthetized with 100 mg/Kg ketamine+10 mg/ml xylazine via i.p. injection. Prior to incision, the abdominal hair was removed with a shaving blade. Buprenex (0.1 mg/Kg body weight) was administered via subcutaneous injection prior to the creation of the incision. Then, the peritoneal cavity of the mouse was accessed through a midline incision. After exposing the duodenum and visualization of the common bile duct, the needle (31G) of the syringe containing 50 μl of PBS or AAV vectors was inserted into the common bile duct adjacent to the duodenum toward the papilla of vater. Then the duodenum tissue surrounding the needle was clamped with a curved micro hemp clip, which was to secure the needle in place and to prevent the injected solution from entering the duodenum. Then the solution in the syringe was slowly injected into the pancreatic duct. After removal of the needle, a cotton swab was held to the injection site to prevent leakage for 10-15 seconds. Then the abdominal organs were carefully placed in position, the peritoneal cavity and the skin sutured with absorbable suture. The mouse was placed on a heating pad for recovery from anesthesia, and returned to its cage following recovery from anesthesia.

Glucose Tolerance Test (GTT). GTT was performed essentially the same as described previously. Zhang et al. (2019). Specifically, following overnight fasting, the mice were injected with 1 g glucose/Kg bodyweight via i.p. injection. Blood glucose was measured prior to glucose injection (time 0), and at various time points after injection (15, 30, 45, 60, 90 and 120 minutes) using AlphaTRAK glucose meter.

Glucose-stimulated insulin secretion (GSIS). In vivo GSIS was performed in the same setting as GTT except that blood was collected for insulin measurement. Zhang et al. (2019). Specifically, after overnight fasting, the mice were injected with 1 g glucose/Kg bodyweight via i.p. injection. About 20 μl blood was collected from tail vein prior to glucose injection (time 0), and at 15 and 30 minutes after glucose injection. Insulin concentrations in the serum were measured using ultrasensitive mouse insulin ELISA kit (from ALPCO) following the manufacturer's instruction.

Hormone measurements. Hormones including mouse insulin, glucagon, and C-peptide were measured using corresponding ELISA kits from ALPCO (Salem, New Hampshire), as described previously (Parajuli et al., Zhang et al. (2016), Zhang et al. (2019)). Specifically, mouse insulin was measured with the mouse ultrasensitive insulin kit 80-INSMSU-E01, mouse C-peptide with the kit 80-CPTMS-E01, and glucagon with the kit 48-GLUHU-E01. All assays followed the manufacturer's protocols. Note the glucagon kit can be used to measure both human and mouse glucagon according to the manufacturer's instruction.

Immunohistochemistry (IHC) staining of pancreatic tissues. This was performed as described previously. Parajuli et al. Specifically, the mouse pancreases were fixed in 10% buffered formalin solution overnight at room temperature, and then processed for paraffin embedding and sectioning. For IHC staining, the pancreatic slices were first deparaffinized with sequential incubation in xylene (4×2 minutes), 100% ethanol (2×1 minute), 95% ethanol (30 seconds), 70% ethanol (45 seconds) and deionized water (1 minute). Antigen retrieval (optional) was then conducted by boiling the pancreas slices in citrate buffer (pH 6.0) for 20 minutes, followed by natural cooling down to below 40° C., and washing with deionized water. (Note: Antigen retrieval was required for Pax4 staining, but not for insulin, glucagon or GFP staining). Prior to antibody incubation, the tissue slices were permeabilized with 0.25% Triton X-100 in PBS for 30 minutes, and blocked in blocking solution (2% Glycine, 2% Bovine Serum Albumin, 5% FBS, 50 mM NH₄Cl in PBS), for 1 hour, at room temperature. The slices were then incubated with the primary antibody diluted in 3% FBS in PBS, overnight at 4° C., followed by incubation with the corresponding secondary antibody that were conjugated to various fluorescent agents. For nuclear staining, Hoechst 33342 (2 μg/ml in PBS) was added to the slides after secondary antibody incubation, and incubated for 10 minutes at room temperature. The slices were then washed in PBS and water, air-dried, mounted with glass coverslip, and processed for fluorescence microscopy. Images were taken with Photometrics IRIS 15 digital camera that was attached to a Nikon Ti-S/L100 Inverted microscope. NIS-Elements Imaging software was used to collect fluorescence images.

Terminal blood collection via cardiac puncture: At approximately 3 months after surgery, blood (1.2 ml) was collected from the mice via cardiac puncture under anesthesia. The procedure resulted in the death of the mice.

Tissue Collection: Pancreas, liver, kidney, intestine, and spleen were harvested, and preserved in 10% formalin solution.

Immunohistochemistry: Formalin-fixed pancreatic tissues were embedded in paraffin, sectioned, and processed for IHC staining of insulin, glucagon, and Pax4 (or GFP) to assess α and β cell condition, and transgene (Pax4 or GFP) expression.

Statistical Evaluation: The statistical analyses were performed using the GraphPad Prism 9 software. When analyzing glucose levels among multiple groups over the time (two parameters, as in GTT and GSIS), two-way ANOVA was performed to determine the significance of their differences. For each data point (at a fixed time point), one-way ANOVA was performed to determine the significance of differences among 3 or more groups, and Student's t-test was used to compare the differences between two groups. All quantitative data were expressed as Mean±SEM. P<0.05 is considered statistically significant. Statistical significance is defined as *:p<0.05, **:p<0.01, ***:p<0.001.

### Results

The STZ-induced T1D mice (5×65 mg/kg bodyweight) developed hyperglycemia at a significantly faster than expected rate compared to previous STZ dosing experience with STZ at 5×50 mg/kg bodyweight, where diabetes developed over a two week period. rAAV vectors were administered when blood glucose levels reached 300 mg/dL. For the mice that were treated within 1 week of last STZ dose, it is possible that STZ-induced β cell death was still ongoing, which could have complicated assessment of the therapeutic effects of the test articles (Goyal et al. and Szkudelski). Data from the mice that developed hyperglycemia after 1 week and thus treated later were analyzed separately (FIG. 6).

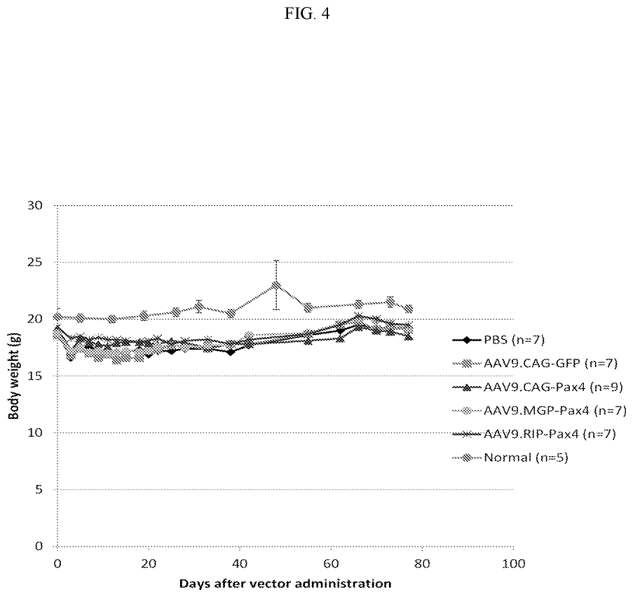

Body weight. The body weight of the mice was monitored throughout the study. There were no significant differences among the treatment groups (FIG. 4). The diabetic mice (treated with test or control articles) weighed less than the normal mice because they lost weight during STZ-induction and subsequent IBD surgery.

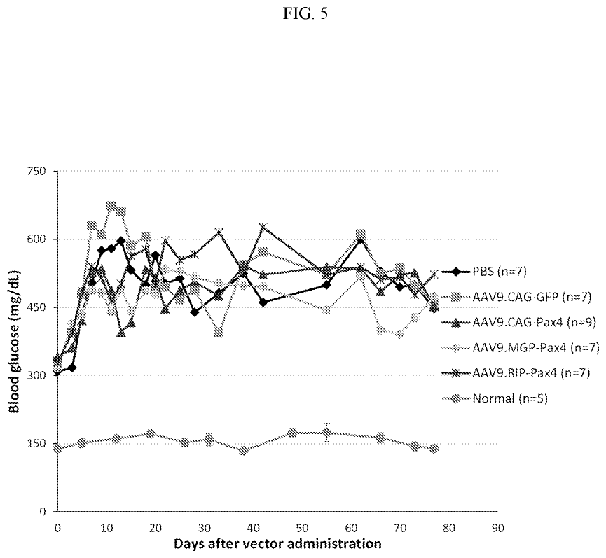

Blood glucose (non-fasting). Non-fasting blood glucose (BG) of each mouse was monitored for ˜12 weeks after IBD injection of the test articles and controls. The mean data of all mice in each group were plotted (FIG. 5). Overall, no significant differences were detected among the treatment groups. Data from the mice that developed hyperglycemia after 1 week and thus treated later were analyzed separately as shown in FIG. 6. Within this group of mice, AAV9.CAG-Pax4 and AAV9.MGP-Pax4 treated T1D mice showed substantially lower BG than the control-treated groups, especially during the first 2-3 weeks after vector administration. Statistical significance of the differences was reached between AAV9.CAG-Pax4-treated mice and both control groups (PBS and AAV9.CAG-GFP), whereas AAV9.MGP-Pax4-treated mice showed significant difference from AAV9.CAG-GFP group (p=0.0327), but not from PBS control group (p=0.0973), probably due to insufficient sample size (n=2).

Glucose Tolerance Test (GTT). The T1D mice in PBS, AAV9.CAG-GFP and AAV9.Pax4 treatment groups, on average, had non-fasting BG of >450 mg/dL (FIG. 5), suggesting they had very poor blood glucose control and wouldn't tolerate glucose challenge well. Nonetheless, GTT was attempted at approximately 8 weeks after vector administration. To perform GTT, mice were fasted overnight. GTT was assessed in the mice treated at least 1 week after last STZ treatment (see FIGS. 7A and 7B). The data show that, compared to the control PBS or GFP groups, AAV9.CAG-Pax4 significantly improved glucose tolerance; AAV9.MGP-Pax4 showed improvement, but did not reach statistical significance probably due to the small sample size. AAV9.RIP-Pax4 did not show any benefit, which may have been due to diabetic mice having very few β cells left, and, thus, the vector couldn't exert its β cell protection effect. Parajuli et al. *: p<0.05, **: p<0.01 between the indicated groups.

Glucose Stimulated Insulin Secretion (GSIS) assay. At about 10 weeks after vector administration, a GSIS assay was performed on the mice that had IBD injection more than 1 week after the last STZ treatment. The mice were fasted overnight, then injected with 1 g/Kg bodyweight of glucose via i.p. injection. Blood was collected before (time 0) glucose injection, and 15 and 30 minutes after glucose injection. Zhang et al. (2019) Insulin concentrations were measured using ultra-sensitive mouse insulin ELISA kits. As shown in FIGS. 8A and 8B, all Pax4-expressing AAV9 vectors-treated mice tended to have more insulin secretion upon glucose injection than the PBS and GFP control groups, suggesting they had more functional β cells, although the differences are not statistically significant due to small sample sizes.

Circulating insulin, glucagon and C-peptide in the mice. At the end of animal study (approximately 3 months after vector administration via IBD injection), blood was collected from each mouse via cardiac puncture, and processed for hormone measurements using mouse insulin, C-peptide and glucagon ELISA kits (ALPCO). Shown in FIGS. 9A-9C are the data from all mice, and no significant differences were detected among different groups for any of the three hormones.

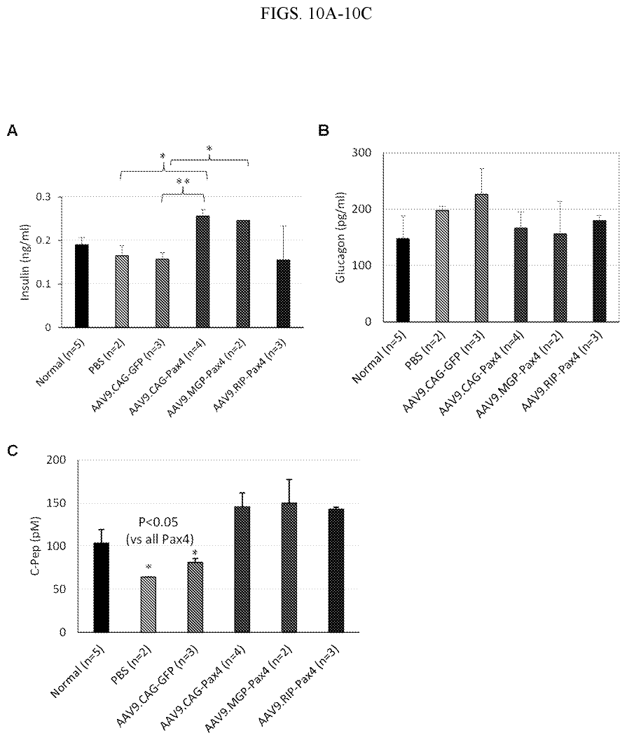

Hormone levels (insulin, glucagon and C-peptide) were also analyzed at the end of the study (3 months after vector administration) from the mice that were treated with vectors and controls one week after the last STZ dose. Some significant differences were detected (FIGS. 10A-10C). Specifically, AAV9.CAG-Pax4-treated mice had significantly more insulin than the control PBS or AAV9.CAG-GFP control treatments (FIG. 10A). All Pax4-containing treatment groups showed significantly more C-peptide than the control treatments (FIG. 10C). Since C-peptide has longer half-life in circulation, circulating C-peptide reflects accumulative β cell function, and higher circulating C-peptide indicates more functional β cells. Note: normal mice didn't show higher insulin or C-peptide than the diabetic groups because they had normal blood glucose, and thus their β cells were not stimulated at the experimental setting. Moreover, circulating glucagon levels in all groups did not show any significant differences (FIG. 10B), suggesting that glucagon deficiency was not induced by the treatments.

Immunohistochemistry assessment of islet α and β cells, and transgene (Pax4, GFP) expression. After the mice were euthanized, their pancreases were harvested and processed for IHC staining of insulin, glucagon, and Pax4 (or GFP). GFP staining showed highly efficient gene delivery into pancreas by AAV9 vector. Pax4 gene expression is detectable, but the number of Pax4⁺ cells was very few compared to GFP⁺ cells, even for the AAV9.CAG-Pax4-treated group. Data not shown. The sensitivity of the Pax4 antibody appeared to be very low, and could only detect cells with high levels of Pax4 expression. Nonetheless, the results show Pax4 expression sustained for 3 months after one dose of vector administration via IBD injection.

### DISCUSSION

The study was designed to examine the therapeutic effects of AAV9-based Pax4 gene delivery in STZ-induced T1D mice following direct vector administration into pancreas via IBD injection. Some results support that Pax4 gene delivery into pancreatic islets increases insulin secretion and improves blood glucose control in T1D.

The T1D induced in the mice by multiple-low doses of STZ (5×65 mg/kg bodyweight) developed hyperglycemia more quickly and severely than observed in prior experience with this model (Goyal et al.). 1/3 of the mice (15/45) had blood glucose (BG) of >300 mg/dL within 5 days of last STZ dose, and more than 50% mice (24/45) reached >300 mg/dL within 1 week of last STZ dose. Vectors were administered when BG reached 300 mg/dL. In the mice that reached the target BG levels prior to 1 week after last STZ dose, STZ-induced β cell death may have been ongoing at the time of vector administration, which could complicate assessment of therapeutic benefit of the constructs. In addition, acute β cell death in the severe T1D models could have left very few residual β cells to be protected by Pax4 expression. Indeed, for data analysis, analyzing only those mice reaching the target BG and hyperglycemia after 1 week, the benefits of Pax4 gene therapy were detected as assessed by GTT, GSIS, and basal hormone secretion (insulin, C-peptide and glucagon) in circulation.

Among the vectors, AAV9.CAG-Pax4 and AAV9.MGP-Pax4 showed the most significant improvements, followed by AAV9.RIP-Pax4. Not to be bound by any particular theory, the MGP promoter would be expected to drive Pax4 gene expression selectively in a cells and not β cells, and, thus, would induce β cell regeneration (by inducing α-to-β cell transdifferentiation); the RIP promoter would be expected to drive Pax4 gene expression selectively in β cells, and, thus would only protect pre-existing β cells; and the ubiquitously active CAG promoter, in contrast, would be expected to drive Pax4 gene expression in both α and β cells (and other cells), and, thus not only promote β cell regeneration from a cells, but also protect pre-existing β cells. The results are consistent. It should be noted that AAV9.CAG-Pax4 can mediate Pax4 expression even in non-pancreatic islet transduced cells due to the ubiquitous nature of the CAG promoter in combination with the wide biodistribution of AAV9.

The dosage of AAV9 vectors used in this study is 3×10¹¹ vg/mouse, which appeared to be sufficient based on GFP gene delivery mediated by AAV9.CAG-GFP vector. The data also demonstrate that AAV9-mediated gene expression is persistent—it lasted for 3 months with one dose. The efficiency of Pax4 gene delivery could not be reliably evaluated because of the sensitivity of Pax4 detection.

### CONCLUSIONS

Single administration of AAV9.Pax4 vectors, especially AAV9.CAG-Pax4 and AAV9.MGP-Pax4, improved blood glucose control as assessed by non-fasting blood glucose monitoring and glucose tolerance test. Second, single administration of AAV9.Pax4 vectors resulted in sustained improvements in β cell function as assessed by GSIS assay, and by basal insulin and C-peptide levels in circulation 3 months after the treatment. These improvements reached statistical significance even with the very small sample size (n=2-4).

### REFERENCES

All publications, patent applications, patents, and other references mentioned herein (e.g., sequence database reference numbers) are incorporated by reference in their entirety. For example, all GenBank, Unigene, and Entrez sequences referred to herein are incorporated by reference. Unless otherwise specified, the sequence accession numbers specified herein, including in any Table herein, refer to the database entries current as of the filing date of this application. When one gene or protein references a plurality of sequence accession numbers, all of the sequence variants are encompassed.
1. Parajuli K R, Zhang Y, Cao A M, Wang H, Fonseca V A, Wu H. Pax4 Gene Delivery Improves Islet Transplantation Efficacy by Promoting beta Cell Survival and alpha-to-beta Cell Transdifferentiation. Cell Transplant. 2020; 29:963689720958655.
2. Lorenzo P I, Juarez-Vicente F, Cobo-Vuilleumier N, Garcia-Dominguez M, Gauthier B R. The Diabetes-Linked Transcription Factor PAX4: From Gene to Functional Consequences. Genes. 2017; 8(3).
3. Brun T, Gauthier B R. A focus on the role of Pax4 in mature pancreatic islet beta-cell expansion and survival in health and disease. Journal of molecular endocrinology. 2008; 40(2):37-45.
4. Lorenzo P I, Fuente-Martin E, Brun T, Cobo-Vuilleumier N, Jimenez-Moreno C M, I G H G, Lopez Noriega L, Mellado-Gil J M, Martin-Montalvo A, Soria B, Gauthier B R. PAX4 Defines an Expandable beta-Cell Subpopulation in the Adult Pancreatic Islet. Scientific reports. 2015; 5:15672.
5. Sosa-Pineda B, Chowdhury K, Torres M, Oliver G, Gruss P. The Pax4 gene is essential for differentiation of insulin-producing beta cells in the mammalian pancreas. Nature. 1997; 386(6623):399-402.
6. Collombat P, Xu X, Ravassard P, Sosa-Pineda B, Dussaud S, Billestrup N, Madsen O D, Serup P, Heimberg H, Mansouri A. The ectopic expression of Pax4 in the mouse pancreas converts progenitor cells into alpha and subsequently beta cells. Cell. 2009; 138(3):449-462.
7. Zhang Y, Fava G E, Wang H, Mauvais-Jarvis F, Fonseca V A, Wu H. PAX4 Gene Transfer Induces alpha-to-beta Cell Phenotypic Conversion and Confers Therapeutic Benefits for Diabetes Treatment. Molecular therapy: the journal of the American Society of Gene Therapy. 2016; 24(2):251-260.
8. Mendell J R, Al-Zaidy S A, Rodino-Klapac L R, Goodspeed K, Gray S J, Kay C N, Boye S L, Boye S E, George L A, Salabarria S, Corti M, Byrne B J, Tremblay J P. Current Clinical Applications of In Vivo Gene Therapy with AAVs. Molecular therapy: the journal of the American Society of Gene Therapy. 2021; 29(2):464-488.
9. Inagaki K, Fuess S, Storm T A, Gibson G A, McTiernan C F, Kay M A, Nakai H. Robust systemic transduction with AAV9 vectors in mice: efficient global cardiac gene transfer superior to that of AAV8. Molecular therapy: the journal of the American Society of Gene Therapy. 2006; 14(1):45-53.
10. Jimenez V, Ayuso E, Mallol C, Agudo J, Casellas A, Obach M, Munoz S, Salavert A, Bosch F. In vivo genetic engineering of murine pancreatic beta cells mediated by single-stranded adeno-associated viral vectors of serotypes 6, 8 and 9. Diabetologia. 2011; 54(5):1075-1086.
11. Bone R N, Icyuz M, Zhang Y, Zhang Y, Cui W, Wang H, Peng J B, Matthews Q L, Siegal G P, Wu H. Gene transfer of active Akt1 by an infectivity-enhanced adenovirus impacts beta-cell survival and proliferation differentially in vitro and in vivo. Islets. 2012; 4(6):366-378.
12. Zhang Y, Parajuli K R, Fava G E, Gupta R, Xu W, Nguyen L U, Zakaria A F, Fonseca V A, Wang H, Mauvais-Jarvis F, Sloop K W, Wu H. GLP-1 Receptor in Pancreatic alpha-Cells Regulates Glucagon Secretion in a Glucose-Dependent Bidirectional Manner. Diabetes. 2019; 68(1):34-44.
13. Goyal S N, Reddy N M, Patil K R, Nakhate K T, Ojha S, Patil C R, Agrawal Y O. Challenges and issues with streptozotocin-induced diabetes—A clinically relevant animal model to understand the diabetes pathogenesis and evaluate therapeutics. Chem Biol Interact. 2016; 244:49-63.
14. Szkudelski T. The mechanism of alloxan and streptozotocin action in B cells of the rat pancreas. Physiol Res. 2001; 50(6):537-546.
15. Furuyama K, Chera S, van Gurp L, Oropeza D, Ghila L, Damond N, Vethe H, Paulo J A, Joosten A M, Berney T, Bosco D, Dorrell C, Grompe M, Raeder H, Roep B O, Thorel F, Herrera P L. Diabetes relief in mice by glucose-sensing insulin-secreting human alpha-cells. Nature. 2019; 567(7746):43-48.
16. Xiao X, Guo P, Shiota C, Zhang T, Coudriet G M, Fischbach S, Prasadan K, Fusco J, Ramachandran S, Witkowski P, Piganelli J D, Gittes G K. Endogenous Reprogramming of Alpha Cells into Beta Cells, Induced by Viral Gene Therapy, Reverses Autoimmune Diabetes. Cell stem cell. 2018; 22(1):78-90 e74.

We claim:

1. A recombinant adeno-associated virus (AAV) vector comprising an expression cassette comprising a nucleotide sequence encoding hPax4, operably linked to a promoter that promotes expression of the nucleotide sequence encoding hPax4 in pancreatic islet cells and a polyadenylation (poly(A)) signal sequence; the promoter comprising a CAG promoter, a rodent or human glucagon promoter, or a rodent or human insulin promoter; wherein the expression cassette is flanked by inverted terminal repeat (ITR) nucleotide sequences.

2. The recombinant AAV vector of claim 1 in which the hPax4 comprises the amino acid sequence of SEQ ID NO: 1.

3. The recombinant AAV vector of claims 1 or 2 in which the nucleic acid that encodes hPax4 comprises a nucleotide sequence having at least 85% identity to the nucleotide sequence of SEQ ID NO: 2 or the reverse complement thereof.

4. The recombinant AAV vector of any one of claims 1 to 3 in which the nucleic acid that encodes hPax4 comprises or consists of the nucleotide sequence of SEQ ID NO: 2 or the reverse complement thereof.

5. The recombinant AAV vector of any one of claims 1 to 4 wherein the promoter has the nucleotide sequence of SEQ ID NO: 4 (CAG promoter), SEQ ID NO: 5 (Mouse Glucagon Promoter) or SEQ ID NO: 6 (Rat Insulin Promoter).

6. The recombinant AAV vector of any one of claims 1 to 5 wherein the poly A signal sequence is an SV40 polyadenylation signal sequence having a nucleotide sequence of SEQ ID NO: 7, or the reverse complement thereof.

7. The recombinant AAV vector of any one of claims 1 to 6 which is a self-complementary vector.

8. The recombinant AAV vector of any one of claims 1 to 7 wherein the ITR nucleotide sequences comprise a 5′ ITR having a nucleotide sequence of SEQ ID NO: 8 and a 3′ ITR having a nucleotide sequence of SEQ ID NO: 25 or a modified 3′ ITR having a nucleotide sequence of SEQ ID NO: 9, or the reverse complement thereof, or a modified 5′ITR sequence and a 3′ ITR sequence having a nucleotide sequence of SEQ ID NO: 25, or the reverse complement thereof.

9. The recombinant AAV vector of any one of claims 1 to 8 in which the expression cassette has the nucleotide sequence of SEQ ID NO: 10 (CAT-hPax4), SEQ ID NO: 11 (MGP-hPax4), or SEQ ID NO: 12 (RIP-hPax4), or the reverse complement thereof.

10. The recombinant AAV vector of any one of claims 1 to 9 comprising the nucleotide sequence of SEQ ID NO: 13 (scAAV-CAG-hPax), SEQ ID NO: 14 (scAAV-MGP-Pax4) or SEQ ID NO: 15 (scAAV-RIP-hPax), or the reverse complement thereof.

11. A recombinant AAV virion comprising: 1) an AAV capsid comprising an AAV capsid protein and having a tropism for pancreatic islet cells; and 2) the recombinant AAV vector of any one of claims 1-10.

12. The AAV virion of claim 11, wherein the AAV capsid protein has an amino acid sequence at least 85% identical to an AAV6 capsid protein (SEQ ID NO: 19), and AAV8 capsid protein (SEQ ID NO: 23), or an AAV9 capsid protein (SEQ ID NO: 21).

13. The AAV virion of claim 12, wherein the AAV capsid protein has the amino acid sequence of SEQ ID NO: 19, 21 or 23.

14. A composition for use in a subject for increasing insulin production in a subject in need thereof, the composition comprising a therapeutically effective amount of the AAV virion of any one of claims 11 to 13 such that the nucleotide sequence encoding hPax4 is expressed in pancreatic islet cells when administered to the subject.

15. A composition for use in a subject for increasing the numbers of β-cells in a subject in need thereof, the composition comprising a therapeutically effective amount of the AAV virion of any one of claims 11-13 such that the nucleotide sequence encoding hPax4 is expressed in pancreatic islet cells when administered to the subject.

16. A composition for use in a subject for treating diabetes mellitus in a subject suffering therefrom, the composition comprising a therapeutically effective amount of the AAV virion of any one of claims 11 to 13 such that the nucleotide sequence encoding hPax4 is expressed in pancreatic islet cells when administered to the subject.

17. The composition for use in claim 16, where the diabetes mellitus is Type 1 diabetes.

18. The composition for use in claim 16, where the diabetes mellitus is Type 2 diabetes.

19. A composition for use in a subject for reducing the risk of developing diabetes in a subject positive for anti-islet autoantibodies, the composition comprising a therapeutically effective amount of the AAV virion of any one of claims 11 to 13 such that the nucleotide sequence encoding hPax4 is expressed in pancreatic islet cells when administered to the subject.

20. The composition for use in any one of claims 14 to 19, wherein said composition is administered by systemic administration, intravenous administration, intramuscular administration, intraperitoneal administration, or subcutaneous administration.

21. The composition for use in any one of claims 14 to 19, wherein said composition is administered by direct administration to the pancreas, intraductal administration or endoscopic retrograde cholangio-pancreatography (ERCP).

22. The composition for use in any one of claims 14 to 21, wherein the composition further comprises a suitable carrier.

23. The composition for use in any one of claims 14 to 22, wherein the subject is a human.

24. An AAV vector plasmid comprising 1) an origin of replication and 2) the recombinant AAV vector of any one of claims 1 to 10.

25. The AAV vector plasmid of claim 24 comprising the nucleotide sequence of SEQ ID NO: 16, SEQ ID NO: 17, or SEQ ID NO: 18.

26. An isolated cell comprising an AAV vector plasmid of claim 24 or 25 and a second plasmid comprising nucleotide sequences encoding rep and cap; the cap encoding a VP1, a VP2, and a VP3; the rep encoding rep78, rep68, rep 52, and rep 40.

27. The cell of claim 26, the cap being AAV6 cap or AAV8 cap or AAV9 cap.

28. A method of producing an AAV virion, the method comprising culturing a host cell comprising the AAV vector plasmid of claim 24 or 25, a second plasmid encoding the cap and rep; the cap encoding the VP1, the VP2, and the VP3; the rep encoding rep78, rep68, rep 52, and rep 40; and any additional adenoviral helper functions, under conditions sufficient to produce the AAV virion.

29. The method of claim 28, further comprising isolating the AAV virion produced by the host cell.


SEQUENCE LISTING (25 sequences)

SEQ ID NO: 1 - protein, 343 aa, synthetic construct
MNQLGGLFVNGRPLPLDTRQQIVRLAVSGMRPCDISRILKVSNGCVSKILGRYYRTGVLEPKGIGGSKPRLATPPVVARIAQLKGECPALFAWEIQRQLCAEGLCTQDKTPSVSSINRVLRALQEDQGLPCTRLRSPAVLAPAVLTPHSGSETPRGTHPGTGHRNRTIFSPSQAEALEKEFQRGQYPDSVARGKLATATSLPEDTVRVWFSNRRAKWRRQEKLKWEMQLPGASQGLTVPRVAPGIISAQQSPGSVPTAALPALEPLGPSCYQLCWATAPERCLSDTPPKACLKPCWGHLPPQPNSLDSGLLCLPCPSSHCPLASLSGSQALLWPGCPLLYGLE

SEQ ID NO: 2 - DNA, 1032 bp, synthetic construct
  source 1..1032: mol_type = other DNA
atgaaccagcttggggggctctttgtgaatggccggcccctgcctctggatacccggcagcagattgtgcggctagcagtcagtggaatgcggccctgtgacatctcacggatccttaaggtatctaatggctgtgtgagcaagatcctagggcgttactaccgcacaggtgtcttggagccaaagggcattgggggaagcaagccacggctggctacaccccctgtggtggctcgaattgcccagctgaagggtgagtgtccagccctctttgcctgggaaatccaacgccagctttgtgctgaagggctttgcacccaggacaagactcccagtgtctcctccatcaaccgagtcctgcgggcattacaggaggaccagggactaccgtgcacacggctcaggtcaccagctgttttggctccagctgtcctcactccccatagtggctctgagactccccggggtacccacccagggaccggccaccggaatcggactatcttctccccaagccaggcagaggcactggagaaagagttccagcgtgggcagtatcctgattcagtggcccgtggaaagctggctactgccacctctctgcctgaggacacggtgagggtctggttttccaacagaagagccaaatggcgtcggcaagagaagctcaagtgggaaatgcagctgccaggtgcttcccaggggctgactgtaccaagggttgccccaggaatcatctctgcacagcagtcccctggcagtgtgcccacagcagccctgcctgccctggaaccactgggtccctcctgctatcagctgtgctgggcaacagcaccagaaaggtgtctgagtgacaccccacctaaagcctgtctcaagccctgctggggccacttgcccccacagccgaattccctggactcaggactgctttgccttccttgcccttcctcccactgtcccctggccagtcttagtggctctcaggccctgctctggcctggctgcccactactgtatggcttggaatga

SEQ ID NO: 3 - skipped

SEQ ID NO: 4 - DNA, 670 bp, synthetic construct
  source 1..670: mol_type = other DNA
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttgg

SEQ ID NO: 5 - DNA, 425 bp, synthetic construct
  source 1..425: mol_type = other DNA
gagatatagccaaataccaaatcaagggataagaccctcaaatgagactaggctcatttgacgtcaaaattcacttgagagaactttagcagtttttcgtgcctgactgagaccgaagggtggatctccaaactgccctttccattcccaaacagaaaggcacaagagtaaataaaatgtttccgggcctctgcggtctcaacccggtatcagcgtaaaaagcagatgagcaaagtgagtgggcgagtgaaatcatttgaacaaaaccccattatttacagatgagaaatttatattgtcagcgtaatatctgcaaggctaaacagcctggagagcatataaaagcacagcaccctggtgcagaagggcagagcttgggcccaggacacactcaaagttcccaaggggactccctctgtctagat

SEQ ID NO: 6 - DNA, 413 bp, synthetic construct
  source 1..413: mol_type = other DNA
gctgagctaagaatccagctatcaatagaaactatgaaacagttccagggacaaagataccaggtccccaacaactgcaactttctgggaaatgaggtggaaaatgctcagccaaggaaaaagagggccttaccctctctgggacaatgattgtgctgtgaactgcttcatcaggccatctggccccttgttaataatctaattaccctaggtctaagtagagttgttgacgtccaatgagcgctttctgcagacttagcactaggcaagtgtttggaaattacagcttcagcccctctcgccatctgcctacctacccctcctagagcccttaatgggccaaacggcaaagtccagggggcagagaggaggtgctttggactataaagctagtggagacccagtaactccca

SEQ ID NO: 7 - DNA, 122 bp, synthetic construct
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 8 - DNA, 145 bp, synthetic construct
  source 1..145: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 9 - DNA, 106 bp, synthetic construct
  source 1..106: mol_type = other DNA
ccactccctctctgcgcgctcgctcgctcactgaggccgggcgaccaaaggtcgcccgacgcccgggctttgcccgggcggcctcagtgagcgagcgagcgcgcag

SEQ ID NO: 10 - DNA, 1869 bp, synthetic construct
  source 1..1869: mol_type = other DNA
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggagatctggtaccgtcgacgccaccatgaaccagcttggggggctctttgtgaatggccggcccctgcctctggatacccggcagcagattgtgcggctagcagtcagtggaatgcggccctgtgacatctcacggatccttaaggtatctaatggctgtgtgagcaagatcctagggcgttactaccgcacaggtgtcttggagccaaagggcattgggggaagcaagccacggctggctacaccccctgtggtggctcgaattgcccagctgaagggtgagtgtccagccctctttgcctgggaaatccaacgccagctttgtgctgaagggctttgcacccaggacaagactcccagtgtctcctccatcaaccgagtcctgcgggcattacaggaggaccagggactaccgtgcacacggctcaggtcaccagctgttttggctccagctgtcctcactccccatagtggctctgagactccccggggtacccacccagggaccggccaccggaatcggactatcttctccccaagccaggcagaggcactggagaaagagttccagcgtgggcagtatcctgattcagtggcccgtggaaagctggctactgccacctctctgcctgaggacacggtgagggtctggttttccaacagaagagccaaatggcgtcggcaagagaagctcaagtgggaaatgcagctgccaggtgcttcccaggggctgactgtaccaagggttgccccaggaatcatctctgcacagcagtcccctggcagtgtgcccacagcagccctgcctgccctggaaccactgggtccctcctgctatcagctgtgctgggcaacagcaccagaaaggtgtctgagtgacaccccacctaaagcctgtctcaagccctgctggggccacttgcccccacagccgaattccctggactcaggactgctttgccttccttgcccttcctcccactgtcccctggccagtcttagtggctctcaggccctgctctggcctggctgcccactactgtatggcttggaatgaagcgctgctcgagagatctacaacttgtttattgcagcttataatggttacaaataaagcaatagcatcacaaatttcacaaataaagcatttttttcactgcattctagttgtggtttgtccaaactcatcaatgtatctta

SEQ ID NO: 11 - DNA, 1622 bp, synthetic construct
  source 1..1622: mol_type = other DNA
gagatatagccaaataccaaatcaagggataagaccctcaaatgagactaggctcatttgacgtcaaaattcacttgagagaactttagcagtttttcgtgcctgactgagaccgaagggtggatctccaaactgccctttccattcccaaacagaaaggcacaagagtaaataaaatgtttccgggcctctgcggtctcaacccggtatcagcgtaaaaagcagatgagcaaagtgagtgggcgagtgaaatcatttgaacaaaaccccattatttacagatgagaaatttatattgtcagcgtaatatctgcaaggctaaacagcctggagagcatataaaagcacagcaccctggtgcagaagggcagagcttgggcccaggacacactcaaagttcccaaggggactccctctgtctagatatctggtaccgtcgacgccaccatgaaccagcttggggggctctttgtgaatggccggcccctgcctctggatacccggcagcagattgtgcggctagcagtcagtggaatgcggccctgtgacatctcacggatccttaaggtatctaatggctgtgtgagcaagatcctagggcgttactaccgcacaggtgtcttggagccaaagggcattgggggaagcaagccacggctggctacaccccctgtggtggctcgaattgcccagctgaagggtgagtgtccagccctctttgcctgggaaatccaacgccagctttgtgctgaagggctttgcacccaggacaagactcccagtgtctcctccatcaaccgagtcctgcgggcattacaggaggaccagggactaccgtgcacacggctcaggtcaccagctgttttggctccagctgtcctcactccccatagtggctctgagactccccggggtacccacccagggaccggccaccggaatcggactatcttctccccaagccaggcagaggcactggagaaagagttccagcgtgggcagtatcctgattcagtggcccgtggaaagctggctactgccacctctctgcctgaggacacggtgagggtctggttttccaacagaagagccaaatggcgtcggcaagagaagctcaagtgggaaatgcagctgccaggtgcttcccaggggctgactgtaccaagggttgccccaggaatcatctctgcacagcagtcccctggcagtgtgcccacagcagccctgcctgccctggaaccactgggtccctcctgctatcagctgtgctgggcaacagcaccagaaaggtgtctgagtgacaccccacctaaagcctgtctcaagccctgctggggccacttgcccccacagccgaattccctggactcaggactgctttgccttccttgcccttcctcccactgtcccctggccagtcttagtggctctcaggccctgctctggcctggctgcccactactgtatggcttggaatgaagcgctgctcgagagatctacaacttgtttattgcagcttataatggttacaaataaagcaatagcatcacaaatttcacaaataaagcatttttttcactgcattctagttgtggtttgtccaaactcatcaatgtatctta

SEQ ID NO: 12 - DNA, 1612 bp, synthetic construct
  source 1..1612: mol_type = other DNA
gctgagctaagaatccagctatcaatagaaactatgaaacagttccagggacaaagataccaggtccccaacaactgcaactttctgggaaatgaggtggaaaatgctcagccaaggaaaaagagggccttaccctctctgggacaatgattgtgctgtgaactgcttcatcaggccatctggccccttgttaataatctaattaccctaggtctaagtagagttgttgacgtccaatgagcgctttctgcagacttagcactaggcaagtgtttggaaattacagcttcagcccctctcgccatctgcctacctacccctcctagagcccttaatgggccaaacggcaaagtccagggggcagagaggaggtgctttggactataaagctagtggagacccagtaactcccaagatctggtaccgtcgacgccaccatgaaccagcttggggggctctttgtgaatggccggcccctgcctctggatacccggcagcagattgtgcggctagcagtcagtggaatgcggccctgtgacatctcacggatccttaaggtatctaatggctgtgtgagcaagatcctagggcgttactaccgcacaggtgtcttggagccaaagggcattgggggaagcaagccacggctggctacaccccctgtggtggctcgaattgcccagctgaagggtgagtgtccagccctctttgcctgggaaatccaacgccagctttgtgctgaagggctttgcacccaggacaagactcccagtgtctcctccatcaaccgagtcctgcgggcattacaggaggaccagggactaccgtgcacacggctcaggtcaccagctgttttggctccagctgtcctcactccccatagtggctctgagactccccggggtacccacccagggaccggccaccggaatcggactatcttctccccaagccaggcagaggcactggagaaagagttccagcgtgggcagtatcctgattcagtggcccgtggaaagctggctactgccacctctctgcctgaggacacggtgagggtctggttttccaacagaagagccaaatggcgtcggcaagagaagctcaagtgggaaatgcagctgccaggtgcttcccaggggctgactgtaccaagggttgccccaggaatcatctctgcacagcagtcccctggcagtgtgcccacagcagccctgcctgccctggaaccactgggtccctcctgctatcagctgtgctgggcaacagcaccagaaaggtgtctgagtgacaccccacctaaagcctgtctcaagccctgctggggccacttgcccccacagccgaattccctggactcaggactgctttgccttccttgcccttcctcccactgtcccctggccagtcttagtggctctcaggccctgctctggcctggctgcccactactgtatggcttggaatgaagcgctgctcgagagatctacaacttgtttattgcagcttataatggttacaaataaagcaatagcatcacaaatttcacaaataaagcatttttttcactgcattctagttgtggtttgtccaaactcatcaatgtatctta

SEQ ID NO: 13 - DNA, 2196 bp, synthetic construct
  source 1..2196: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatgtaatacgactcactagttcctggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggagatctggtaccgtcgacgccaccatgaaccagcttggggggctctttgtgaatggccggcccctgcctctggatacccggcagcagattgtgcggctagcagtcagtggaatgcggccctgtgacatctcacggatccttaaggtatctaatggctgtgtgagcaagatcctagggcgttactaccgcacaggtgtcttggagccaaagggcattgggggaagcaagccacggctggctacaccccctgtggtggctcgaattgcccagctgaagggtgagtgtccagccctctttgcctgggaaatccaacgccagctttgtgctgaagggctttgcacccaggacaagactcccagtgtctcctccatcaaccgagtcctgcgggcattacaggaggaccagggactaccgtgcacacggctcaggtcaccagctgttttggctccagctgtcctcactccccatagtggctctgagactccccggggtacccacccagggaccggccaccggaatcggactatcttctccccaagccaggcagaggcactggagaaagagttccagcgtgggcagtatcctgattcagtggcccgtggaaagctggctactgccacctctctgcctgaggacacggtgagggtctggttttccaacagaagagccaaatggcgtcggcaagagaagctcaagtgggaaatgcagctgccaggtgcttcccaggggctgactgtaccaagggttgccccaggaatcatctctgcacagcagtcccctggcagtgtgcccacagcagccctgcctgccctggaaccactgggtccctcctgctatcagctgtgctgggcaacagcaccagaaaggtgtctgagtgacaccccacctaaagcctgtctcaagccctgctggggccacttgcccccacagccgaattccctggactcaggactgctttgccttccttgcccttcctcccactgtcccctggccagtcttagtggctctcaggccctgctctggcctggctgcccactactgtatggcttggaatgaagcgctgctcgagagatctacaacttgtttattgcagcttataatggttacaaataaagcaatagcatcacaaatttcacaaataaagcatttttttcactgcattctagttgtggtttgtccaaactcatcaatgtatcttaaggcctaggtgagctctggtaccctctccactccctctctgcgcgctcgctcgctcactgaggccgggcgaccaaaggtcgcccgacgcccgggctttgcccgggcggcctcagtgagcgagcgagcgcgcag

SEQ ID NO: 14 - DNA, 1949 bp, synthetic construct
  source 1..1949: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatgtaatacgactcactagttcctggaggggtggagtcgtgacctagggagatatagccaaataccaaatcaagggataagaccctcaaatgagactaggctcatttgacgtcaaaattcacttgagagaactttagcagtttttcgtgcctgactgagaccgaagggtggatctccaaactgccctttccattcccaaacagaaaggcacaagagtaaataaaatgtttccgggcctctgcggtctcaacccggtatcagcgtaaaaagcagatgagcaaagtgagtgggcgagtgaaatcatttgaacaaaaccccattatttacagatgagaaatttatattgtcagcgtaatatctgcaaggctaaacagcctggagagcatataaaagcacagcaccctggtgcagaagggcagagcttgggcccaggacacactcaaagttcccaaggggactccctctgtctagatatctggtaccgtcgacgccaccatgaaccagcttggggggctctttgtgaatggccggcccctgcctctggatacccggcagcagattgtgcggctagcagtcagtggaatgcggccctgtgacatctcacggatccttaaggtatctaatggctgtgtgagcaagatcctagggcgttactaccgcacaggtgtcttggagccaaagggcattgggggaagcaagccacggctggctacaccccctgtggtggctcgaattgcccagctgaagggtgagtgtccagccctctttgcctgggaaatccaacgccagctttgtgctgaagggctttgcacccaggacaagactcccagtgtctcctccatcaaccgagtcctgcgggcattacaggaggaccagggactaccgtgcacacggctcaggtcaccagctgttttggctccagctgtcctcactccccatagtggctctgagactccccggggtacccacccagggaccggccaccggaatcggactatcttctccccaagccaggcagaggcactggagaaagagttccagcgtgggcagtatcctgattcagtggcccgtggaaagctggctactgccacctctctgcctgaggacacggtgagggtctggttttccaacagaagagccaaatggcgtcggcaagagaagctcaagtgggaaatgcagctgccaggtgcttcccaggggctgactgtaccaagggttgccccaggaatcatctctgcacagcagtcccctggcagtgtgcccacagcagccctgcctgccctggaaccactgggtccctcctgctatcagctgtgctgggcaacagcaccagaaaggtgtctgagtgacaccccacctaaagcctgtctcaagccctgctggggccacttgcccccacagccgaattccctggactcaggactgctttgccttccttgcccttcctcccactgtcccctggccagtcttagtggctctcaggccctgctctggcctggctgcccactactgtatggcttggaatgaagcgctgctcgagagatctacaacttgtttattgcagcttataatggttacaaataaagcaatagcatcacaaatttcacaaataaagcatttttttcactgcattctagttgtggtttgtccaaactcatcaatgtatcttaaggcctaggtgagctctggtaccctctccactccctctctgcgcgctcgctcgctcactgaggccgggcgaccaaaggtcgcccgacgcccgggctttgcccgggcggcctcagtgagcgagcgagcgcgcag

SEQ ID NO: 15 - DNA, 1939 bp, synthetic construct
  source 1..1939: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatgtaatacgactcactagttcctggaggggtggagtcgtgacctagggctgagctaagaatccagctatcaatagaaactatgaaacagttccagggacaaagataccaggtccccaacaactgcaactttctgggaaatgaggtggaaaatgctcagccaaggaaaaagagggccttaccctctctgggacaatgattgtgctgtgaactgcttcatcaggccatctggccccttgttaataatctaattaccctaggtctaagtagagttgttgacgtccaatgagcgctttctgcagacttagcactaggcaagtgtttggaaattacagcttcagcccctctcgccatctgcctacctacccctcctagagcccttaatgggccaaacggcaaagtccagggggcagagaggaggtgctttggactataaagctagtggagacccagtaactcccaagatctggtaccgtcgacgccaccatgaaccagcttggggggctctttgtgaatggccggcccctgcctctggatacccggcagcagattgtgcggctagcagtcagtggaatgcggccctgtgacatctcacggatccttaaggtatctaatggctgtgtgagcaagatcctagggcgttactaccgcacaggtgtcttggagccaaagggcattgggggaagcaagccacggctggctacaccccctgtggtggctcgaattgcccagctgaagggtgagtgtccagccctctttgcctgggaaatccaacgccagctttgtgctgaagggctttgcacccaggacaagactcccagtgtctcctccatcaaccgagtcctgcgggcattacaggaggaccagggactaccgtgcacacggctcaggtcaccagctgttttggctccagctgtcctcactccccatagtggctctgagactccccggggtacccacccagggaccggccaccggaatcggactatcttctccccaagccaggcagaggcactggagaaagagttccagcgtgggcagtatcctgattcagtggcccgtggaaagctggctactgccacctctctgcctgaggacacggtgagggtctggttttccaacagaagagccaaatggcgtcggcaagagaagctcaagtgggaaatgcagctgccaggtgcttcccaggggctgactgtaccaagggttgccccaggaatcatctctgcacagcagtcccctggcagtgtgcccacagcagccctgcctgccctggaaccactgggtccctcctgctatcagctgtgctgggcaacagcaccagaaaggtgtctgagtgacaccccacctaaagcctgtctcaagccctgctggggccacttgcccccacagccgaattccctggactcaggactgctttgccttccttgcccttcctcccactgtcccctggccagtcttagtggctctcaggccctgctctggcctggctgcccactactgtatggcttggaatgaagcgctgctcgagagatctacaacttgtttattgcagcttataatggttacaaataaagcaatagcatcacaaatttcacaaataaagcatttttttcactgcattctagttgtggtttgtccaaactcatcaatgtatcttaaggcctaggtgagctctggtaccctctccactccctctctgcgcgctcgctcgctcactgaggccgggcgaccaaaggtcgcccgacgcccgggctttgcccgggcggcctcagtgagcgagcgagcgcgcag

SEQ ID NO: 16 - DNA, 5755 bp, synthetic construct
  source 1..5755: mol_type = other DNA
ggcgcgcccaaaggctatcaggtcattgcctgagagtctggagcaaacaagagaatcgttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatgtaatacgactcactagttcctggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggagatctggtaccgtcgacgccaccatgaaccagcttggggggctctttgtgaatggccggcccctgcctctggatacccggcagcagattgtgcggctagcagtcagtggaatgcggccctgtgacatctcacggatccttaaggtatctaatggctgtgtgagcaagatcctagggcgttactaccgcacaggtgtcttggagccaaagggcattgggggaagcaagccacggctggctacaccccctgtggtggctcgaattgcccagctgaagggtgagtgtccagccctctttgcctgggaaatccaacgccagctttgtgctgaagggctttgcacccaggacaagactcccagtgtctcctccatcaaccgagtcctgcgggcattacaggaggaccagggactaccgtgcacacggctcaggtcaccagctgttttggctccagctgtcctcactccccatagtggctctgagactccccggggtacccacccagggaccggccaccggaatcggactatcttctccccaagccaggcagaggcactggagaaagagttccagcgtgggcagtatcctgattcagtggcccgtggaaagctggctactgccacctctctgcctgaggacacggtgagggtctggttttccaacagaagagccaaatggcgtcggcaagagaagctcaagtgggaaatgcagctgccaggtgcttcccaggggctgactgtaccaagggttgccccaggaatcatctctgcacagcagtcccctggcagtgtgcccacagcagccctgcctgccctggaaccactgggtccctcctgctatcagctgtgctgggcaacagcaccagaaaggtgtctgagtgacaccccacctaaagcctgtctcaagccctgctggggccacttgcccccacagccgaattccctggactcaggactgctttgccttccttgcccttcctcccactgtcccctggccagtcttagtggctctcaggccctgctctggcctggctgcccactactgtatggcttggaatgaagcgctgctcgagagatctacaacttgtttattgcagcttataatggttacaaataaagcaatagcatcacaaatttcacaaataaagcatttttttcactgcattctagttgtggtttgtccaaactcatcaatgtatcttaaggcctaggtgagctctggtaccctctccactccctctctgcgcgctcgctcgctcactgaggccgggcgaccaaaggtcgcccgacgcccgggctttgcccgggcggcctcagtgagcgagcgagcgcgcagggcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacttaattaacatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagta

SEQ ID NO: 17 - DNA, 5508 bp, synthetic construct
  source 1..5508: mol_type = other DNA
ggcgcgcccaaaggctatcaggtcattgcctgagagtctggagcaaacaagagaatcgttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatgtaatacgactcactagttcctggaggggtggagtcgtgacctagggagatatagccaaataccaaatcaagggataagaccctcaaatgagactaggctcatttgacgtcaaaattcacttgagagaactttagcagtttttcgtgcctgactgagaccgaagggtggatctccaaactgccctttccattcccaaacagaaaggcacaagagtaaataaaatgtttccgggcctctgcggtctcaacccggtatcagcgtaaaaagcagatgagcaaagtgagtgggcgagtgaaatcatttgaacaaaaccccattatttacagatgagaaatttatattgtcagcgtaatatctgcaaggctaaacagcctggagagcatataaaagcacagcaccctggtgcagaagggcagagcttgggcccaggacacactcaaagttcccaaggggactccctctgtctagatatctggtaccgtcgacgccaccatgaaccagcttggggggctctttgtgaatggccggcccctgcctctggatacccggcagcagattgtgcggctagcagtcagtggaatgcggccctgtgacatctcacggatccttaaggtatctaatggctgtgtgagcaagatcctagggcgttactaccgcacaggtgtcttggagccaaagggcattgggggaagcaagccacggctggctacaccccctgtggtggctcgaattgcccagctgaagggtgagtgtccagccctctttgcctgggaaatccaacgccagctttgtgctgaagggctttgcacccaggacaagactcccagtgtctcctccatcaaccgagtcctgcgggcattacaggaggaccagggactaccgtgcacacggctcaggtcaccagctgttttggctccagctgtcctcactccccatagtggctctgagactccccggggtacccacccagggaccggccaccggaatcggactatcttctccccaagccaggcagaggcactggagaaagagttccagcgtgggcagtatcctgattcagtggcccgtggaaagctggctactgccacctctctgcctgaggacacggtgagggtctggttttccaacagaagagccaaatggcgtcggcaagagaagctcaagtgggaaatgcagctgccaggtgcttcccaggggctgactgtaccaagggttgccccaggaatcatctctgcacagcagtcccctggcagtgtgcccacagcagccctgcctgccctggaaccactgggtccctcctgctatcagctgtgctgggcaacagcaccagaaaggtgtctgagtgacaccccacctaaagcctgtctcaagccctgctggggccacttgcccccacagccgaattccctggactcaggactgctttgccttccttgcccttcctcccactgtcccctggccagtcttagtggctctcaggccctgctctggcctggctgcccactactgtatggcttggaatgaagcgctgctcgagagatctacaacttgtttattgcagcttataatggttacaaataaagcaatagcatcacaaatttcacaaataaagcatttttttcactgcattctagttgtggtttgtccaaactcatcaatgtatcttaaggcctaggtgagctctggtaccctctccactccctctctgcgcgctcgctcgctcactgaggccgggcgaccaaaggtcgcccgacgcccgggctttgcccgggcggcctcagtgagcgagcgagcgcgcagggcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacttaattaacatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagta

SEQ ID NO: 18 - DNA, 5498 bp, synthetic construct
  source 1..5498: mol_type = other DNA
ggcgcgcccaaaggctatcaggtcattgcctgagagtctggagcaaacaagagaatcgttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatgtaatacgactcactagttcctggaggggtggagtcgtgacctagggctgagctaagaatccagctatcaatagaaactatgaaacagttccagggacaaagataccaggtccccaacaactgcaactttctgggaaatgaggtggaaaatgctcagccaaggaaaaagagggccttaccctctctgggacaatgattgtgctgtgaactgcttcatcaggccatctggccccttgttaataatctaattaccctaggtctaagtagagttgttgacgtccaatgagcgctttctgcagacttagcactaggcaagtgtttggaaattacagcttcagcccctctcgccatctgcctacctacccctcctagagcccttaatgggccaaacggcaaagtccagggggcagagaggaggtgctttggactataaagctagtggagacccagtaactcccaagatctggtaccgtcgacgccaccatgaaccagcttggggggctctttgtgaatggccggcccctgcctctggatacccggcagcagattgtgcggctagcagtcagtggaatgcggccctgtgacatctcacggatccttaaggtatctaatggctgtgtgagcaagatcctagggcgttactaccgcacaggtgtcttggagccaaagggcattgggggaagcaagccacggctggctacaccccctgtggtggctcgaattgcccagctgaagggtgagtgtccagccctctttgcctgggaaatccaacgccagctttgtgctgaagggctttgcacccaggacaagactcccagtgtctcctccatcaaccgagtcctgcgggcattacaggaggaccagggactaccgtgcacacggctcaggtcaccagctgttttggctccagctgtcctcactccccatagtggctctgagactccccggggtacccacccagggaccggccaccggaatcggactatcttctccccaagccaggcagaggcactggagaaagagttccagcgtgggcagtatcctgattcagtggcccgtggaaagctggctactgccacctctctgcctgaggacacggtgagggtctggttttccaacagaagagccaaatggcgtcggcaagagaagctcaagtgggaaatgcagctgccaggtgcttcccaggggctgactgtaccaagggttgccccaggaatcatctctgcacagcagtcccctggcagtgtgcccacagcagccctgcctgccctggaaccactgggtccctcctgctatcagctgtgctgggcaacagcaccagaaaggtgtctgagtgacaccccacctaaagcctgtctcaagccctgctggggccacttgcccccacagccgaattccctggactcaggactgctttgccttccttgcccttcctcccactgtcccctggccagtcttagtggctctcaggccctgctctggcctggctgcccactactgtatggcttggaatgaagcgctgctcgagagatctacaacttgtttattgcagcttataatggttacaaataaagcaatagcatcacaaatttcacaaataaagcatttttttcactgcattctagttgtggtttgtccaaactcatcaatgtatcttaaggcctaggtgagctctggtaccctctccactccctctctgcgcgctcgctcgctcactgaggccgggcgaccaaaggtcgcccgacgcccgggctttgcccgggcggcctcagtgagcgagcgagcgcgcagggcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacttaattaacatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagta

SEQ ID NO: 19 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 20 - DNA, 2211 bp, synthetic construct
  source 1..2211: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 21 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 22 - DNA, 2211 bp, synthetic construct
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 23 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 24 - DNA, 4393 bp, synthetic construct
  source 1..4393: mol_type = other DNA
cagagagggagtggccaactccatcactaggggtagcgcgaagcgcctcccacgctgccgcgtcagcgctgacgtaaattacgtcataggggagtggtcctgtattagctgtcacgtgagtgcttttgcggcattttgcgacaccacgtggccatttgaggtatatatggccgagtgagcgagcaggatctccattttgaccgcgaaatttgaacgagcagcagccatgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccaatggcgccgcgtgagtaaggccccggaggccctcttctttgttcagttcgagaagggcgagagctactttcacctgcacgttctggtcgagaccacgggggtcaagtccatggtgctaggccgcttcctgagtcagattcgggaaaagcttggtccagaccatctacccgcggggtcgagccccaccttgcccaactggttcgcggtgaccaaagacgcggtaatggcgccggcgggggggaacaaggtggtggacgagtgctacatccccaactacctcctgcccaagactcagcccgagctgcagtgggcgtggactaacatggaggagtatataagcgcgtgcttgaacctggccgagcgcaaacggctcgtggcgcagcacctgacccacgtcagccagacgcaggagcagaacaaggagaatctgaaccccaattctgacgcgcccgtgatcaggtcaaaaacctccgcgcgctatatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattacccagaaccgcatctaccgcatcctcgctctcaacggctacgaccctgcctacgccggctccgtctttctcggctgggctcagaaaaagttcgggaaacgcaacaccatctggctgtttggacccgccaccaccggcaagaccaacattgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagcctctccaggaccggatgtttaagttcgaactcacccgccgtctggagcacgactttggcaaggtgacaaagcaggaagtcaaagagttcttccgctgggccagtgatcacgtgaccgaggtggcgcatgagttttacgtcagaaagggcggagccagcaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacgtgcgagagaatgaatcagaatttcaacatttgcttcacacacggggtcagagactgctcagagtgtttccccggcgtgtcagaatctcaaccggtcgtcagaaagaggacgtatcggaaactctgtgcgattcatcatctgctggggcgggctcccgagattgcttgctcggcctgcgatctggtcaacgtggacctggatgactgtgtttctgagcaataaatgacttaaaccaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcg

SEQ ID NO: 25 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa